(19) United States
(12) Patent Application Publication
(10) Pub. No.: US 2026/0315292 A1
(43) Pub. Date: Oct. 15, 2026

(54) DISPOSABLE ABSORBENT PANT-TYPE ARTICLE

(71) Applicant: Essity Hygiene and Health Aktiebolag, Göteborg (SE)

(72) Inventors: Hans EEN, Mölnlycke (SE); Lucas BÄCK, Göteborg (SE); Rickard LÖNNROTH, Göteborg (SE)

(73) Assignee: Essity Hygiene and Health Aktiebolag, Göteborg (SE)

(21) Appl. No.: 19/485,405

(22) PCT Filed: Jun. 12, 2024
(86) PCT No.: PCT/EP2024/066214

§ 371 Date: Nov. 18, 2025

(30) Foreign Application Priority Data
Jun. 13, 2023 (WO) PCT/EP2023/065835

(51) Int. Cl. A61F 13/49; A61F 13/15; A61F 13/496

(52) U.S. Cl. CPC A61F 13/49011; A61F 13/15593; A61F 13/496

(57) ABSTRACT

The present disclosure relates to a disposable absorbent pant-type article having longitudinal and transversal extensions. The article includes front, back, and crotch sections. An absorbent assembly is arranged with a central part located in the crotch section. At least one of the front and back sections includes first and second leg elastic members arranged along leg edges, wherein each leg elastic member includes one or more elastic elements extending in the longitudinal direction. The first and second leg elastic members are sandwiched between first layer bonded to a second in at least one of the front and back sections. At least one of the front and back sections includes a set of elastic strands extending in the transversal direction. The first and second leg elastic members end at or in the vicinity of the leg edges. The present disclosure also relates to a method for producing the article.

### TECHNICAL FIELD

The present disclosure pertains to a disposable absorbent pant-type article comprising: a front section and, a back section, and a crotch section located between the front section and the back section. The present disclosure also relates to a method for producing a disposable absorbent pant-type article.

The present disclosure further relates to a laminate web material and to a method for forming a laminate web material for at least one of a front section or a back section of a disposable absorbent pant-type article.

### BACKGROUND

In the art of disposable absorbent pant-type articles, and in particular disposable absorbent pant-type articles which are intended for use by adult users, the articles should meet high demands on fit, comfort and have a tailored and underwear like appearance while at the same time meeting equally high demands on absorbency and leakage protection. Disposable absorbent articles are articles which are intended to be discarded after a single use and not be laundered or otherwise restored for repeated use.

Pant-type articles of this kind are relatively complex products involving multiple components which are combined in high-speed processes to form the absorbent article. Such components include nonwoven components, barrier components, elastic components and usually an absorbent core which may be provided in the form of an absorbent assembly including a barrier layer and a liquid permeable topsheet which together enclose the absorbent core.

Threaded elastic laminates, formed by sandwiching pre-tensioned elastomeric threads between layers of web materials, e.g., non-woven web materials, are used to create pant-type articles with body-conforming properties resembling those of high-quality textile underwear. In addition, it is common to enhance fit and leakage security by arranging pre-tensioned elastic elements along all or selected parts of the waist opening and leg openings of the pant-type article.

Numerous methods are known for providing threaded elastic laminates. Adhesive bonding methods involve application of adhesive to bond the pre-tensioned elastomeric threads between the layers of web materials. Welding bonding methods involve the application of a weld bond pattern to bond the pre-tensioned elastomeric threads between the layers of web materials. The weld bonds may be produced by heat bonding or ultrasonic bonding. Moreover, regardless of the bonding method used, requirements for breathability, adaptability and softness of the threaded laminates imparts other requirements to the bonding method, e.g., regarding the size and distribution of the bonds.

In addition, it is known to provide elastic members along waist openings and leg openings of the disposable absorbent pant-type articles, in order to meet high demands on fit and comfort, while at the same time meet equally high demands on absorbency and leakage protection.

In order to produce the disposable absorbent pant-type articles at an industrial scale, it is desirable that the elastic components can be provided in a cost-efficient manner and at high production speeds, as well as the article as a whole being appropriate for manufacturing at high production speeds in a cost-efficient manner.

Accordingly, there exists an ongoing need for improved or alternative threaded elastic laminates in a disposable absorbent patent-type article and for improved or alternative methods for forming such a threaded elastic laminate as well as methods for manufacturing disposable absorbent patent-type articles.

### SUMMARY

In a first aspect, the need may be fulfilled by a disposable absorbent pant-type article according to claim 1. As such, there is provided a disposable absorbent pant-type article, such as a pant diaper, a sanitary pant or incontinence pant. The article has a longitudinal extension along a longitudinal direction and a transversal extension along a transversal direction. The article comprises a front section, a back section, and a crotch section, which is located between and joining the front section and the back section. An absorbent assembly is arranged with a central part of the absorbent assembly being located in the crotch section. The front section is joined to the back section by a first front section side edge being joined to a first back section side edge in a first side seam, and a second front section side edge being joined to a second back section side edge in a second side seam, such that the article forms a waist opening, defined by a waist edge of the front section and a waist edge of the back section, a first leg opening, defined by a first leg edge of the front section and a first leg edge of the back section, and a second leg opening, defined by a second leg edge of the front section and a second leg edge of the back section. At least one of the front section and the back section comprises a first leg elastic member, arranged along the first leg edge of the at least one of the front section and the back section, and a second leg elastic member, arranged along the second leg edge of the at least one of the front section and the back section, wherein each leg elastic member, comprises one or more elastic elements extending at least partially in the longitudinal direction. The first leg elastic member ends at or in the vicinity of the first leg edge, and the second leg elastic member ends at or in the vicinity of the second leg edge.

The disposable absorbent pant-type article may be an adult incontinence article, a baby diaper, training pants, swim-pants, disposable underpants, a sanitary pant for feminine use, etc. Disposable absorbent articles are articles which are intended to be discarded after a single use and not be laundered or otherwise restored for repeated use. In the present disclosure, the disposable absorbent pant-type article is sometimes referred to as “the pant-type article” or just “the article”.

The article has a longitudinal extension along a longitudinal direction and a transversal extension along a transversal direction. These two directions are perpendicular to each other. Any measurements and percentages of distances and dimensions are given assuming the article is in a fully stretched-out, non-gathered condition, i.e. when it is in an unfolded and flat condition.

The front section and the back section may be formed by separate panels, i.e. a front body panel and a rear body panel. In that case, the crotch region may be formed by a portion of the absorbent assembly, such that the portion of the absorbent assembly spans the gap between the front body panel and the rear body panel.

Alternatively, the front section, the back section and the crotch section may be made of a chassis forming a single piece. It would also be possible to form the front section and the crotch section as a single piece and have a separate panel for the back section or to form the back section and the crotch section in a single piece and have a separate panel for the front section.

Optionally, the front section and the back section are formed to comprise no more than two layers of sheet material, which may be referred to as bottom layer and top layer.

In some variants, the bottom layer and the top layer of the at least one of the front section and the back section is/are formed by a respective single continuous material sheet. That no additional layers of web material are present may be advantageous in terms of breathability, comfort, sustainability, and economics. However, it may also be feasible that the at least one of the front section and the back section is formed by a combined panel of the type described herein.

The absorbent assembly typically comprises an absorbent core. In that case, the absorbent assembly may comprise the absorbent core, a liquid permeable topsheet and a liquid impermeable backsheet, wherein the absorbent core typically is sandwiched between the liquid permeable topsheet and the liquid impermeable backsheet. The absorbent core may have any useful shape, such as hourglass shape, rectangular, T-shape, etc., as known in the art.

The side seams may be generally band-shaped joints which are formed by ultrasonic welding or thermowelding. To have sufficient strength to withstand the forces to which the pant-type article is exposed during manufacturing of the article and to allow sufficient production tolerances when cutting off individual pant-type articles from a precursor web, the side seams commonly have a width in the order of 5 to 10 millimetres. It is also known to make side seams having a width of less than 5 mm.

It is generally desired that a soiled pant-type article can be easily removed without having to pull it down over the legs of a user. Therefore, the side seams are commonly made such that they are breakable by manual force to allow a user or a caregiver to pull apart the side seams before removing a soiled pant-type article. The pant-type article may be a reclosable article which means that the side seams can be manually opened e.g., to check if the article is soiled and needs to be changed and then closed again if it is determined that the article is in a condition allowing further use. Reclosability is commonly provided by means of fastener elements, such as hook-and-loop-type fasteners.

At least one of the front section and the back section comprises a first and a second leg elastic member. Hence, the back section only, the front section only or both the back section and the front section may comprise first and the second leg elastic members ending at the or in the vicinity of the leg edges, as disclosed herein.

That the first and second leg elastic member extend at least partially in the longitudinal direction implies that the leg elastic member is at least partially curved or inclined, such that it spans over a longitudinal extension. Each leg elastic member may for example form an angle of at least 10 degrees with the transversal direction, over at least a portion of the longitudinal extension, such as over at least 20% of the longitudinal extension of the leg elastic member. For example, each leg elastic member may form an angle of from 10 to 80 degrees with the transversal direction, over at least a portion of the longitudinal extension of the leg elastic member, such as over at least 20% of the longitudinal extension of the leg elastic member. Each leg elastic member may hence be a curved leg elastic member, such as is generally known in the art of absorbent articles.

The leg elastic member comprises one or more elastic elements, e.g. in the form of a string, thread, strand or band. As used herein the terms “elastic” and “elastomeric” are used in parallel. Each leg elastic member may comprise a plurality of individual elastic elements, such as 2 to 15 individual elastic elements, 2 to 10 individual elastic element or 2 to 6 individual elastic elements. The individual elastic elements may be applied spaced apart from each other along the leg edges. The individual elastic elements may be located at equal distances from each other or may have varying interspaces. Typically, the individual elastic elements in the at least one of the front section and the back section are attached in a stretched condition. Accordingly, in a relaxed state, they contract, typically causing local wrinkles in the at least one of the front section and the back section.

The first leg elastic member ends at or in the vicinity of the first leg edge, and the second leg elastic member ends at or in the vicinity of the second leg edge. Thereby the end of the respective leg elastic member may be located at the very edge of the leg edge, e.g. if the leg elastic member is fully adhered to the material of the at least one of the front section and the back section. Alternatively, the end of the respective leg elastic member may be slightly retracted from the very edge of the leg edge, such as within a range of between 0 and 20 mm, or 3 and 10 mm, due to the pull-back effect of the elasticity when the pulling force used during manufacturing of the disposable absorbent pant-type article is released. The first leg elastic member and the second leg elastic member are typically mirror-images of each other.

Hence, the first and the second leg elastic member has a respective end that is located at or in the vicinity of the respective leg edge. These ends of the first and the second leg elastic member are facing away from the absorbent assembly, i.e. these ends are distant from the absorbent assembly. As described herein, the first and the second leg elastic member may also have a respective end facing the absorbent assembly, i.e. these ends are located at or in the vicinity of the absorbent assembly.

The disposable absorbent pant-type articles as disclosed herein may comprise an elastic waist feature. The elastic waist feature may form a continuous waistband encircling the waist opening of the disposable absorbent pant-type article, or may be applied only to selected portions of the waist opening, such as only along central portions of the waist edge of the front and back sections or only along the waist edge of the back section etc.

The elastic waist feature may be completely overlapping with the front section and/or with the back section and may share a waist edge with the corresponding front section and/or back section. The elastic waist feature may be applied to a waist edge of the front section and/or back section and protrude beyond the waist edge of the corresponding front section and/or back section.

The elastic waist feature may comprise one or more elastic elements. The waist feature may comprise a plurality of elastic strands or elastic bands. Alternatively, the elastic waist feature may comprise or consist of a band of elastic material, such as elastic nonwoven, elastic foam or elastic film. The elastic waist feature may be attached to the front section and/or to the back section as a prefabricated component, such as a waistband component comprising two layers of carrier web with elastic strands or bands attached between the layers of carrier web. An elastic waist feature comprising elastic elements, such as strands or bands, may alternatively be formed directly on the front or back section by applying the elastic elements to the front or back section along a waist edge of the front or back section and subsequently covering the elastic elements by a cover web, such as a nonwoven cover web.

In some variants, the elastic waist feature may be a component of the disposable absorbent pant-type article which is distinct from the material of the front section and the back section. In other variants, the elastic waist feature may be an integral waist portion of the front section and/or back section of the absorbent article.

Numerous options for forming an elastic waist feature on a front section and/or on a back section are known and may be combined with the present disclosure. For example, the different variants described in WO 2018/09771 A1 may be used on the front and/or back sections as described herein. For example, the different variants as described in EP 3 788 997 A1 may be used on the front and/or back sections as described herein.

In the disposable absorbent pant-type article according to the invention, the first and the second leg elastic members are independent of the side seam, i.e. they are not included in the side seam. This differs from prior art solutions, in which the leg elastic members end in the side seam and thereby are included in the side seam. Thereby, a shorter length of leg elastic member is used in an article according to the invention as compared to an article of the same size according to prior art. This makes the material cost of the article according to the invention lower.

Further, since a shorter length of leg elastic member is used, it also follows that less adhesive may be used to attach it to the materials of the at least one of the front section and the back section as compared to an article of the same size according to prior art. This makes the adhesive cost of the article according to the invention lower as compared to an article of the same size according to prior art. Further, since there is a risk that an adhesive may undesirably stiffen the materials of the at least one of the front section and the back section, less adhesive will result in a smaller such risk.

Also, in conventional pant-type articles where the leg elastic members are included in the side seam, it is of importance that the adhesive attaching the leg elastic members between the first layer and the second layer is not applied in the region of the side seam. This is because the side seam is preferably an ultrasonically welded seam, and any adhesive in the ultrasonically welded region of the side seam may negatively impact the ultrasonic welding capability due to adhesive contamination on the horn and/or anvil. To accurately control the adhesive application, high demands are set on having accurate control of start and stop of the adhesive application equipment, and also on the exact programming of the equipment made by the operator. By the solution proposed herein, the leg elastics are not included in the side seam. Thus, the risk for adhesive contamination in the side seam welding may be eliminated. Also, the design with the adhesive application may be simplified.

Preferably, the first and second leg elastic members are sandwiched between the first and second layers and bonded by an adhesive located on one or more of the first layer and the second layer. The adhesive may be applied in a pre-selectable pattern over the first and/or second layers. Preferably, the side seams are ultrasonically welded seams. Due to the elimination of the risk of adhesive contamination as explained in the above, the present disclosure allows for particular advantages when the first and second leg elastic members are sandwiched between the first and second layers and bonded by an adhesive located on one or more of the first layer and the second layer, wherein the adhesive is present in a pre-selectable pattern over the first and/or second layers, and the side seams are ultrasonically welded seams. For example, the pre-selectable pattern may be such that adhesive extends to or substantially to the first/second leg edge.

In addition, since the leg elastic members are not included in the side seams, the elastic material of the leg elastic members can be selected, without having to consider that it should be suitable for the joining method used for the side seam.

Optionally, the first leg elastic member and the second leg elastic member end with a respective cut end. Hence, the one or more elastic members have been cut. Thereby, the leg elastic member has a distinct end. Preferably, the cutting of the leg elastic member is performed when making the leg cut during manufacturing of the disposable absorbent pant-type article.

The first and second leg elastic members are sandwiched between a first layer and a second layer comprised in the at least one of the front section and the back section, which first and second layers are bonded to each other. The first layer may form one of a top or a bottom layer of the article. The second layer may form the other of the top and bottom layer of the article. Hence, either the first layer or the second layer may face the wearer of the article.

Optionally, the first and second layers are bonded to each other by an adhesive located on one or more of the first layer, the second layer and the first and second leg elastic members. When located on more than one of the first layer, the second layer and the first and second leg elastic members, the same or different adhesives may be used at the different locations, e.g. having different rates of consolidating. If the adhesive is applied to the first or second layer, the adhesive may be applied in a pre-selectable pattern, and thus not all over the surface.

As mentioned in the above, optionally, the first and second leg elastic members are sandwiched between the first and second layers and bonded by an adhesive located on one or more of the first layer and the second layer. The adhesive may be applied in a pre-selectable pattern over the first and/or second layers.

As an alternative or a complement to adhesive, the first and second layers may be bonded to each other by means of welding, e.g. ultrasonic welding and/or thermowelding.

The at least one of the front section and the back section further comprises a set of elastic strands extending substantially or completely in the transversal direction of the article. The elastic strands of the set may be located at equal distances from each other or may have varying interspaces, e.g. being closer to each other in zones where more elasticity is desired. Typically, the elastic strands of the set are attached between the first and second layers when in a stretched condition. Accordingly, in a relaxed state, the respective first and second layers form wrinkles along the sets of elastic strands.

The at least one of the front or back section is thus extendible from the relaxed state to a stretched-out state, in which the sets of elastic strands are stretched out along the transversal direction causing the wrinkles of the first and second layers to diminish or to completely flatten out.

Optionally, the first and second layers comprised in the at least one of the front section and the back section are bonded to each other by adhesive located on the elastic strands of the set elastic strands and/or by a welded bond pattern associated with the elastic strands of the set elastic strands, the set of elastic strands being sandwiched between the first and second layers. Hence, the adhesive on the set elastic strands and/or the welded bond pattern is an alternative or a complement to the above-mentioned bonding of the first and the second layer to each other.

The welded bond pattern may comprise pairs of adjacent weld bonds for attaching individual strands of the elastic strands of the set using such pairs of adjacent weld bonds, e.g. attaching each strand of the set individually. The welded bonds are preferably arranged such that the elastic strands are mechanically trapped between the weld bonds. For example, each of the elastic strands of the set may be mechanically trapped between the adjacent weld bonds of a plurality of pairs of adjacent weld bonds.

Optionally, at least one of the front section and the back section comprises a first leg top elastic strand and a second leg top elastic strand extending substantially or completely in the transversal direction. The first leg top elastic strand ends at the first leg edge in a longitudinal position being between the first leg elastic member and the first side seam. The second leg top elastic strand ends at the second leg edge in a longitudinal position being between the second leg elastic member and the second side seam. Hence, the first and second top elastic strands are able to provide elasticity in a zone of the respective leg edge being between the end of the leg elastic member and the side seam. The term longitudinal position as used herein refers to a position as seen in the longitudinal direction of the article.

The first leg top elastic strand and the second leg top elastic strand may be formed by a single elastic strand extending from the first leg edge to second leg edge. Alternatively, they may be formed by two separate elastic strands, such that there is a gap between them, e.g. allowing space for the absorbent core.

Optionally, at least one of the front section and the back section comprises a first leg side elastic strand, extending substantially or completely in the transversal direction, which crosses the first leg elastic member, and a second leg side elastic strand, extending substantially or completely in the transversal direction, which crosses the second leg elastic member. Hence, the leg side elastic strand ends at the leg edge further away from the side seam than the leg elastic member.

The first leg side elastic strand and the second side top elastic strand may be formed by a single elastic strand extending from the first leg edge to second leg edge. Alternatively, they may be formed by two separate elastic strands, such that there is a gap between them, e.g. allowing space for the absorbent core.

Optionally, the first and second leg top elastic strands and/or the first and second leg side elastic strands form part of, or constitute, the set of elastic strands extending substantially or completely in the transversal direction of the article. As mentioned above, the individual elastic elements may be located at equal distances from each other or may have varying interspaces. Further they are typically provided in a stretched state when manufacturing the article.

Optionally, the first leg edge has a longitudinal extension as seen in the longitudinal direction of the article, the first leg elastic member ending at a longitudinal position of the first leg edge being located within a range of from 1% to 60%, preferably within a range of from 10% to 50%, more preferably within a range of from 20% to 40% of the longitudinal extension, as determined from a lower end of the first side seam. The second leg edge has a longitudinal extension as seen in the longitudinal direction of the article, typically being the same as the longitudinal extension of the first leg edge. The second leg elastic member ends at a longitudinal position of the second leg edge being located within a range of from 1% to 60%, preferably within a range of from 10% to 50%, more preferably within a range of from 20% to 40%, of the longitudinal extension, as determined from a lower end of the second side seam. Thereby, there is an interspace between the side seam and the leg elastic member. The lower end of the side seam is the end of the side seam being closest to the leg edge. An upper end of the side seam is located at or in the vicinity of the waist feature. The lower end of the side seam is the opposite end of the side seam. Hence, when the wearer of the absorbent article is standing up, the lower end is downwards.

Optionally, the first leg elastic member ends at a longitudinal position being located within a range of from 1 mm to 80 mm, preferably within a range of from 10 mm to 70 mm, more preferably within a range of from 20 mm to 55 mm as determined from the lower end of the first side seam, and the second leg elastic member ends at a longitudinal position of the second leg edge being located within a range from 1 mm to 80 mm, preferably within a range of from 10 mm to 70 mm, more preferably within a range of from 20 mm to 55 mm, as determined from the lower end of the second side seam.

Optionally, the first leg elastic member extends along a distance being within a range 50%-99% of a length of the first leg edge, preferably 55%-95%, more preferably 60%-90%, and the second leg elastic member extends along a distance being within a range 50%-99% of the length of the second leg edge, preferably 55%-95%, more preferably 60%-90%. Thereby, the length of the respective leg edge is measured along the edge itself following its curvature and thus extends both in the longitudinal and the transversal directions.

Optionally, the first leg elastic member extends a distance being within a range of 150-300 mm along the first leg edge, preferably 165-285 mm, more preferably 180-270 mm, and the second leg elastic member extends within a range of 150-300 mm along the first leg edge, preferably 165-285 mm, more preferably 180-270 mm. Thereby, the extension along the respective leg edge is measured along the edge itself following its curvature and thus extends both in the longitudinal and the transversal directions.

Optionally, the first leg elastic member extends at least from the absorbent assembly to the first leg edge and the second leg elastic member extends at least from the absorbent assembly to the second leg edge. The first and second leg elastic member may also extend below at least part of the absorbent assembly, but optionally no leg elastic member extends continuously under the whole width of the absorbent assembly. Thereby, the risk of wrinkling the absorbent assembly, and in particular the absorbent core, by means of a leg elastic members going over the whole width of the absorbent assembly is avoided. This may be achieved by deliberately interrupting the leg elastic member in a central area of the article, e.g. by cutting it, such that a gap is provided for the absorbent assembly. Methods for accomplishing this are described in in WO 2018/106160 A1, to which we refer.

Optionally, the first leg elastic member extends along a longitudinal length being less than 40% preferably less than 35%, more preferably less than 30% of a total longitudinal length of the at least one of the front section and the back section, and the second leg elastic member extends along a longitudinal length being less than 40% preferably less than 35%, more preferably less than 30% of a total longitudinal length of the at least one of the front section and the back section.

Optionally, the disposable absorbent pant-type article comprises a first and a second crotch elastic member, extending along a respective longitudinal side of the absorbent assembly. Thereby, the crotch elastic member will form part of the elasticity around the leg opening.

Optionally, in the disposable absorbent pant-type article according to the invention, the at least one of the front section and the back section comprises a combined panel, wherein the combined panel comprises, as seen along the longitudinal direction, a first panel portion and a second panel portion, the first panel portion and the second panel portion being connected by a connection region. The first panel portion comprises a first bottom layer and a first top layer being superimposed such that, in the first panel portion, the first bottom layer forms a bottom side surface of the combined panel and the first top layer forms a top side surface of the combined panel. The first panel portion further comprises at least a subset of the set of elastic strands being sandwiched between the first bottom layer and the first top layer, the elastic strands being aligned substantially in parallel with each other and with the transversal direction, and the elastic strands being spaced apart from each other in the longitudinal direction. The first panel portion is bonded by the first bottom layer being bonded to the first top layer by a welded bond pattern attaching the at least a subset of the set of elastic strands between the first bottom layer and the first top layer. The second panel portion comprises a second bottom layer and a second top layer being super-imposed such that, in the second panel portion, the second bottom layer forms a bottom side surface of the combined panel and the second top layer forms a top side surface of the combined panel, wherein the first leg elastic member and the second leg elastic member are sandwiched between the second bottom layer and the second top layer, and the second panel portion is bonded by the second bottom layer being bonded to the second top layer by adhesive bonds, attaching the first leg elastic member and the second leg elastic member between the second bottom layer and the second top layer.

The first panel portion further comprises at least a subset of the set of elastic strands of the article. Hence, the elastic strands in the first panel portion can constitute the set of elastic strands of the article, or be comprised in the set of elastic strands of the article. There may be another subset of the set of elastic strands of the article comprised in the second panel portion sandwiched between the second bottom layer and the second top layer. The subset of the first panel portion may differ from the subset of the second panel portion as regards longitudinal spacing between the elastic strands, and/or features of the elastic strands such as diameter of the elastic strands

A second aspect of the present invention relates to a method for forming a laminate web material for at least one of a front section or a back section a laminate web material for a disposable absorbent pant-type article, the laminate web material being continuous as seen in a machine direction and having a width in a cross direction between a first side edge and a second side edge, the method comprising:
S110: Feeding a bottom layer web in the machine direction.
S120: Feeding a top layer web in the machine direction.
S125: Feeding a set of continuous elastic strands in the machine direction.
S130: Feeding a continuous leg elastic member comprising one or more elastic elements.
S140: Positioning the continuous leg elastic member, such that it extends over less than 40% of the width of the laminate web material, preferably less than 35%, more preferably less than 30%.
S150: Bonding the bottom layer web to the top layer web to form the laminate web material, thereby sandwiching the set of continuous elastic strands between the bottom layer web and the top layer web, such that the elastic strands of the set of continuous elastic strands are aligned substantially in parallel with each other and with the machine direction, and being spaced apart from each other in the cross direction and sandwiching the continuous leg elastic member between the bottom layer web and the top layer web.

The terms bottom layer web and the top layer web as used herein refer to the way that they are typically provided in the manufacturing equipment. In the disposable absorbent pant-type article, the bottom layer web may either form the inner side, i.e., the side against the wearer, or the outer side of the article.

The bottom layer web and the top layer web are supplied as continuous in the machine direction. The typically have a basis weight within the range of from 8 to 26 gsm.

The first edge of the laminate web material is the edge from where the leg cuts will be made. Hence, the continuous leg elastic member partly extends along and adjacent to this edge in the first zone. The continuous leg elastic member is applied in an undulating way as seen in the cross direction, i.e. forming an undulating wave. One flat apex of the undulating wave is thus in the first zone, while the other flat apex of the undulating wave is in the second zone. In the first and second zone, the continuous leg elastic member extends parallel with or substantially parallel with the machine direction. The wavelength of the undulating wave corresponds to the width of the disposable absorbent pant-type article as seen in the transverse direction of the article. In between the first and second zone, there is a transfer zone, in which the continuous leg elastic member extends both in the cross direction and the machine direction. Further details of the undulation can be derived from the description herein of the first and second leg elastic members in the disposable absorbent pant-type article. Later on, when absorbent assemblies are applied to the laminate web material, the absorbent assemblies are preferably positioned such that they overlap with the continuous leg elastic member in the first zone. Furthermore, the leg cuts will preferably extend into the second zone.

The laminate web material made with the method may be used as a laminate web material for at least one of a front section or a back section for a disposable absorbent pant-type article as described herein. Hence, details and advantages described herein for the article or its manufacture, are as far as they are applicable, valid also for the laminate web material.

The continuous leg elastic member is positioned such that it extends over less than 40% of the width of the laminate web material, preferably less than 35%, more preferably less than 30%. This facilitates manufacturing a disposable absorbent pant-type article, in which the first leg elastic member ends at or in the vicinity of the first leg edge, and the second leg elastic member ends at or in the vicinity of the second leg edge.

The method comprises:
S125: Feeding a set of continuous elastic strands in the machine direction, wherein step S150 further comprises attaching the set of continuous elastic strands between the bottom layer web and the top layer web, thereby sandwiching the set of continuous elastic strands between the bottom layer web and the top layer web, such that the elastic strands of the set of elastic strands are aligned substantially in parallel with each other and with the machine direction and being spaced apart from each other in the cross direction.

The continuous elastic strands will impart elasticity in the transversal direction of the article.

Optionally, step S140 comprises positioning the continuous leg elastic member, such that it extends for preferably at least 10 mm in a direction parallel with or substantially parallel with the machine direction in a first zone along and adjacent to the first edge of the laminate web material, extends for preferably at least 10 mm in a direction parallel with or substantially parallel with the machine direction in a second zone spaced apart in the machine direction and the cross direction from the first zone, and extends both in the cross direction and the machine direction in a transfer zone located between the first and second zones

Optionally, the method comprises positioning the continuous leg elastic member, such that it crosses at least one elastic strand of the set of continuous elastic strands. The crossed elastic strand may then form a leg side elastic strand of the type described herein.

One or more of steps S110, S120, S125, S130, and S140 may be performed simultaneously or substantially simultaneously. They may also be performed in an arbitrary order.

Optionally, step S150 comprises bonding by applying adhesive to form adhesive bonds and/or by performing welding, e.g. ultrasonic welding and/or thermowelding.

Optionally, step S150 comprises applying adhesive to one or more of the bottom layer web, the top layer web, the continuous leg elastic member and the set of continuous elastic strands. The same or different adhesives may be used at the different locations.

Optionally, step S150 comprises applying adhesive to one or more of the bottom layer web and the top layer web in defined regions overlapping the continuous leg elastic member. The same or different adhesives may be used at the different locations.

Optionally, the method further comprises after step S150:
S160: Making leg cuts going from the first side edge of the laminate web material, thereby cutting through the continuous leg elastic member.

In the method for forming a laminate web material described herein, the laminate web material may be used for forming a front section or back section of a disposable absorbent pant-type article, such as a pant diaper, a sanitary pant or incontinence pant, and the continuous leg elastic member may be applied so as to form leg elastic members in the disposable absorbent pant-type article.

For embodiments of the invention, in which the laminate web material is a combined laminate material web, the combined laminate material web being continuous as seen in a machine direction and having a width in a cross-direction perpendicular to the machine direction between a first side edge and a second side edge, the combined laminate material web having a top side and a bottom side as seen in a thickness direction perpendicular to the machine direction and to the cross-direction, the method may optionally comprise a welding bonding step and an adhesive bonding step, wherein the welding bonding step comprises feeding a first bottom layer web, a first top layer web, and at least a subset of the set of continuous elastic strands in the machine direction, and bonding the first bottom layer web to the first top layer web by a welded bond pattern so as to attach the at least a subset of the set of continuous elastic strands between the first bottom layer web and the first top layer web, the set of elastic strands being aligned in parallel with each other and substantially with the machine direction, and being spaced apart from each other in the cross-direction, and the adhesive bonding step comprises feeding a second top layer web and a second bottom layer web in the machine direction, and feeding the continuous leg elastic member, and bonding the second bottom layer web to the second top layer web by adhesive bonds so as to attach the continuous leg elastic member between the second bottom layer web and the second top layer web, the continuous leg elastic member extending at least partially in a direction not parallel with the machine direction.

This disclosure further comprises a method for forming an absorbent pant-type article, comprising forming a laminate web material according to the second aspect as described herein, and forming at least one of a front section or a back section of an absorbent pant-type article by cutting said laminate web material, the method further comprising making leg cuts going from a first side edge of the laminate web material, thereby cutting through the continuous leg elastic member.

A third aspect of the present invention relates to a laminate web material for a disposable absorbent pant-type article, such as a pant diaper, a sanitary pant or incontinence pant. The laminate web material is continuous as seen in a machine direction and has a width in a cross direction between a first side edge and a second side edge. The laminate web material comprises a bottom layer web, a top layer web, a continuous leg elastic member sandwiched between the bottom layer web and the top layer web, and a set of continuous elastic strands in the machine direction sandwiched between the bottom layer web and the top layer web, the elastic strands of the set of elastic strands being aligned substantially in parallel with each other and with the machine direction, and being spaced apart from each other in the cross direction, wherein the continuous leg elastic member extends over less than 40% of the width of the laminate web material, preferably less than 35%, more preferably less than 30%.

The laminate web material may be used as a laminate web material for at least one of a front section or a back section for a disposable absorbent pant-type article as described herein. Hence, the laminate web material may be seen as an intermediate product when manufacturing the disposable absorbent pant-type article. Details and advantages described herein for the article or its manufacture, are, as far as they are applicable, valid also for the laminate web material. The laminate web material may be manufactured with the method described herein.

The laminate web material further comprises a set of continuous elastic strands in the machine direction sandwiched between the bottom layer web and the top layer web, the elastic strands of the set of elastic strands being aligned substantially in parallel with each other and with the machine direction and being spaced apart from each other in the cross direction.

Optionally, the continuous leg elastic member extends preferably for at least 10 mm in a direction parallel with or substantially parallel with the machine direction in a first zone along and adjacent to the first edge of the laminate web material, preferably for at least 10 mm in a direction parallel with or substantially parallel with the machine direction in a second zone spaced apart from the first zone, and both in the cross direction and the machine direction in a transfer zone located between the first and second zones.

At least one elastic strand of the set of continuous elastic strands may cross the continuous leg elastic member in order to form a leg side elastic strand as described herein for the disposable absorbent pant-type article.

A fourth aspect of the present invention relates to a method for producing a disposable absorbent pant-type article, such as a pant diaper, a sanitary pant or incontinence pant, the article comprising a front section, a back section, and a crotch section being located between the front section and the back section, wherein an absorbent assembly is arranged with a central part of the absorbent assembly being located in the crotch section. The method comprises:
M110: Feeding a bottom layer web in the machine direction,
M120: Feeding a top layer web in the machine direction,
M125: Feeding a set of continuous elastic strands in the machine direction,
M130: Feeding a continuous leg elastic member comprising one or more elastomeric elements,
M140: Positioning the continuous leg elastic member, such that it extends at least partly in an extension direction being both in the cross direction and the machine direction,
M150: Bonding the bottom layer web to the top layer web to form a first laminate web material, thereby sandwiching the set of continuous elastic strands between the bottom layer web and the top layer web, such that the elastic strands of the set of continuous elastic strands are aligned substantially in parallel with each other and with the machine direction, and being spaced apart from each other in the cross direction and sandwiching the continuous leg elastic member between the bottom layer web and the top layer web,
M160: Positioning the first laminate web material in a position corresponding to one of a front section or a back section of the article,
M170: Providing a second laminate web material and positioning the second laminate web material in a position corresponding to the other of the front section or the back section of the article,
M180: Intermittently providing absorbent assemblies spaced apart in the machine direction and positioning them with a first end portion in overlap with the first laminate web material and a second end portion in overlap with the second laminate web material, thereby forming a composite web of interconnected precursor articles,
M190: Making leg cuts going from a first side edge of the first laminate web material, thereby cutting through the continuous leg elastic member, the leg cuts being positioned in between the absorbent assemblies as seen in a machine direction,
M200: Folding the composite web along a fold line extending in the machine direction, thereby bringing a first edge of the composite web into alignment with a second edge of the composite web,
M210: Forming first and second side seams in each precursor article, thereby joining the first laminate web material with the second laminate web material, the side seams being formed in the cross direction along dividing lines between the precursor articles,
M220: Severing individual disposable absorbent pant-type articles from the composite web of precursor articles.

The method may be used to manufacture the disposable absorbent pant-type article according to the invention. The method may include one or more of the steps of the method for forming a laminate web material for at least one of a front section or a back section a laminate web material for a disposable absorbent pant-type article disclosed herein, e.g. including one or more of steps S110-S160.

Steps M110, M120, M125 and M130 corresponds to steps S110, S120, S125 and S130. Please see the comments to steps S110-S130 disclosed herein.

In step M140, the continuous leg elastic member is positioned in such a pattern that the leg cuts made in step M190 will cut through the continuous leg elastic member. Hence, the continuous leg elastic member is typically applied in an undulating way as seen in the cross direction. The wavelength of the undulation corresponds to the width of the disposable absorbent pant-type article as seen in the transverse direction of the article. Further details of the undulation can be derived from the description herein of the first and second leg elastic members in the disposable absorbent pant-type article. Step M140 can be performed as described herein for step S140, but also other patterns for positioning the continuous leg elastic member are feasible, as long as the leg cuts made in step M190 will cut through the continuous leg elastic member.

Step M150 corresponds to S150. Please see the comments to steps S150 disclosed herein. In this step a first laminate web material is formed, which is to form one of the front section or the back section of the disposable absorbent pant-type article, cf. step M160.

In step M170, a second laminate web material is provided, which is to form the other of the front section or the back section of the disposable absorbent pant-type article. The second laminate web material may be formed in the corresponding steps of M110-M150. Alternatively, the second laminate web material may be made in any of a number of ways known from prior art. The second laminate web material may be with or without leg elastic members.

In M180 absorbent assemblies are provided. Each absorbent assembly typically comprises a respective absorbent core. In that case, the absorbent assembly may comprise the absorbent core, a liquid permeable topsheet and a liquid impermeable backsheet, wherein the absorbent core typically is sandwiched between the liquid permeable topsheet and the liquid impermeable backsheet. The absorbent core may have any useful shape, such as hourglass shape, rectangular, T-shape, etc., as known in the art.

The front section and the back section may be formed by separate panels, i.e. a front body panel and a rear body panel. In that case, step M180 comprises that the crotch region is formed by a portion of the absorbent assembly, such that the portion of the absorbent assembly spans the gap between the front body panel and the rear body panel.

Alternatively, the front section, the back section and the crotch section may be made of a chassis forming a single piece. In that case, the first laminate web material and/or second laminate web material may be made or provided as part of the chassis. It would also be possible to form the front section and the crotch section as a single piece and have a separate panel for the back section or to form the back section and the crotch section in a single piece and have a separate panel for the front section.

In step M190, leg cuts are made such that the continuous leg elastic member is cut. Thereby, in the article being manufactured by the method, the first leg elastic member ends at or in the vicinity of the first leg edge, and the second leg elastic member ends at or in the vicinity of the second leg edge.

Step M200 is performed in order to prepare the precursor article for applying the side seams in step M210. Finally, the disposable absorbent pant-type article is separated from the following article in step M220.

Optionally, the method may further comprise cutting or deactivating the continuous leg elastic member so as to form sets of elastic members separated by a non-elastic portion, where the non-elastic portion is intended to be located at the absorbent assembly.

Either the bottom layer web or the top layer web may face the wearer of the article.

The method may further comprise a step of providing a waist feature 50 of the type described herein and/or as known from prior art. This step may e.g. be made just before step M150, in step M150 or just after step M150.

Optionally, step M140 comprises positioning the continuous leg elastic member, such that it extends for preferably at least 10 mm in a direction parallel with or substantially parallel with the machine direction MD in a first zone along and adjacent to the first edge of the laminate web material, for preferably at least 10 mm in a direction parallel with or substantially parallel with the machine direction in a second zone spaced apart from the first zone, and both in the cross direction CD and the machine direction MD in a transfer zone located between the first and second zones.

Thereby, one flat apex of the undulating wave of the continuous leg elastic member may be in the first zone, while the other flat apex of the undulating wave is in the second zone. In the first and second zone, the continuous leg elastic member extends parallel with or substantially parallel with the machine direction. In between the first and second zone, there is a transfer zone, in which the continuous leg elastic member extends both in the cross direction and the machine direction. Later on, when absorbent assemblies are applied to the laminate web material, the absorbent assemblies are preferably positioned such that they overlap with the continuous leg elastic member in the first zone. Furthermore, the leg cuts will preferably extend into the second zone.

Optionally, step M140 comprises positioning the continuous leg elastic member, such that it extends over less than 40% of the width of the first laminate web material, preferably less than 35%, more preferably less than 30%.

The method comprises:
M125: Feeding a set of continuous elastic strands in the machine direction, wherein step M150 comprises attaching the set of continuous elastic strands between the bottom layer web and the top layer web, thereby sandwiching the set of continuous elastic strands between the bottom layer web and the top layer web, such that the elastic strands of the set of elastic strands are aligned substantially in parallel with each other and with the machine direction, and being spaced apart from each other in the cross direction. Please see the comments to step S125.

One or more of steps M110, M120, M125, M130, M140 may be performed simultaneously or substantially simultaneously. They may also be performed in an arbitrary order.

Optionally, step M140 comprises positioning the continuous leg elastic member in relation to the set of continuous strands, such that it crosses at least one elastic strand of the set of continuous elastic strands. The crossed elastic strand may then form a leg side elastic strand of the type described herein.

Optionally, step M140 comprises positioning the continuous leg elastic member in relation to the set of continuous strands, such that in step M190 at least one elastic strand of the set of continuous elastic strands ends at the leg cut without crossing the continuous leg elastic member.

Optionally, step M150 comprises bonding by applying adhesive to form adhesive bonds and/or by performing welding, e.g. ultrasonic welding and/or thermowelding.

Optionally, step M150 comprises applying adhesive to one or more of the bottom layer web, the top layer web, the continuous leg elastic member and the set of continuous elastic strands.

Optionally, step M150 comprises applying adhesive to one or more of the bottom layer web and the top layer web in defined regions overlapping the continuous leg elastic member.

As set out in relation to the first aspect, the present disclosure offers a possibility for reducing or eliminating the risk of adhesive contamination in a method for forming an absorbent article wherein-the step M120 for forming first and second side seams comprises forming said first and second side seams by ultrasonic welding. For example, the the step M150 comprises applying adhesive to one or more of the bottom layer web and the top layer web in defined regions overlapping the continuous leg elastic members, and the step M120 comprises forming said first and second side seams by ultrasonic welding.

As should be understood from the above, the present disclosure relates to an article wherein the front section and/or the back section comprises an elastic threaded laminate, or to methods concerning the forming of an elastic threaded laminate or such an article. In an elastic threaded laminate, the elasticity is primarily obtained by elastomeric strands being sandwiched between first and second layers. This is in contrast to elastic film laminates, where the elasticity may be obtained by an elastic film or other elastic material layer, which may in turn optionally be sandwiched between additional layers. Thus, the front section and/or back section of the articles proposed herein, and the elastic laminate as proposed herein may be free from elastic film.

### Combined Panel Variants

In view of sustainability requirements as well as economic requirements, welding bonding methods may be preferred since such methods do not require the application of an adhesive. However, in in-line continuous production, welding bonding methods have been found to be difficult to adapt to achieve secure attachment of pre-tensioned elements that have curved shapes, such as may be the case for pre-tensioned elastic elements used e.g., along all or selected parts of the leg openings of the pant-type articles.

In variants of the invention disclosed herein, there is provided a disposable absorbent pant-type article for which at least one of the front section or the back section comprises or constitutes a combined panel. As already mentioned herein, the front section and the back section may be formed by separate panels, i.e. a front body panel and a rear body panel.

In such variants of the disposable absorbent pant-type article according to the invention, the article has a longitudinal direction and a transverse direction, and defines a thickness direction perpendicular to the longitudinal direction and the transverse direction, wherein an inside of the article is towards the wearer when the article is worn, and an outside of the article is away from the wearer when the article is worn, the article comprising:
a front body panel,
a rear body panel, and
a crotch region being located between the front body panel and the rear body panel, wherein an absorbent assembly comprising an absorbent core is arranged with a central part of the absorbent core being located in the crotch region.

The front body panel is joined to the rear body panel by a first front panel side edge being joined to a first rear panel side edge in a first side seam, and a second front panel side edge being joined to a second rear panel side edge in a second side seam, such that the article forms a waist opening, defined by a waist edge of the front panel and a waist edge of the rear panel, and a pair of leg openings, defined by a pair of leg edges of the front body panel and a pair of leg edges of the rear body panel. Hence, there is a first and a second leg edge in each of the front body panel and the back body panel.

Each of the front body panel and the rear body panel has a top side and a bottom side as seen in the thickness direction of the article. The terms “top side” and “bottom side” do not refer to any specific orientation in the thickness direction of the article (unless otherwise stated). Instead, the top side of a body panel is one out of the inside and the outside of the body panel, and the bottom side of a body panel is the other one out of the inside and the outside of the body panel.

At least one of the front body panel and rear body panel is a combined panel, which combined panel comprises a pair of leg elastic members arranged adjacent the pair of leg edges of the combined panel, wherein each leg elastic member extends at least partially in a direction other than parallel with the transversal direction. Hence, each pair of leg elastic members, comprises the first and second leg elastic members disclosed herein. Further, the combined panel comprises, as seen along the longitudinal direction, a first panel portion and a second panel portion, the first panel portion and the second panel portion being connected by a connection region. The first panel portion extends from the connection region and in the longitudinal direction towards the waist edge of the combined panel, and the second panel portion extends from the connection region and in a longitudinal direction towards the pair of leg edges of the combined panel. The second panel portion comprises the pair of leg elastic members.

The first panel portion comprises a first bottom layer and a first top layer being superimposed such that, in the first panel portion, the first bottom layer forms a bottom side surface of the combined panel and the first top layer forms a top side surface of the combined panel. The first panel portion further comprises a first set of elastomeric strands being sandwiched between the first bottom layer and the first top layer, the elastomeric strands being aligned substantially in parallel with each other and with the transversal direction, and the elastomeric strands being spaced apart from each other in the longitudinal direction. The first panel portion is bonded by the first bottom layer being bonded to the first top layer by a welded bond pattern attaching the first set of elastomeric strands between the first bottom layer and the first top layer. The first set of elastomeric strands may be a subset or may form the set of elastomeric strands described herein.

The second panel portion comprises a second bottom layer and a second top layer being super-imposed such that, in the second panel portion, the second bottom layer forms a bottom side surface of the combined panel and the second top layer forms a top side surface of the combined panel. The pair of leg elastic members are sandwiched between the second bottom layer and the second top layer, and the second panel portion is bonded by the second bottom layer being bonded to the second top layer by adhesive bonds, attaching the pair of leg elastic members between the second bottom layer and the second top layer.

Optionally, the second panel portion further comprises a second set of elastomeric strands, wherein the elastomeric strands are aligned substantially in parallel with each other and with the transversal direction, and the elastomeric strands are spaced apart from each other in the longitudinal direction. In this case, the second panel portion is bonded by the second bottom layer being bonded to the second top layer by adhesive bonds, attaching the pair of leg elastic members and the second set of elastomeric strands between the second bottom layer and the second top layer. The optional set of elastomeric strands may be comprised in the set of elastomeric strands described herein, e.g. as a subset.

By virtue of the combined panel comprising a first panel portion which is bonded by welded bond pattern and a second panel portion which is bonded by adhesive, it is possible to provide a front or rear body panel which is bonded by adhesive where this is required to enable attachment of the leg elastic members, where each leg elastic member extends at least partially in a direction other than parallel with the transversal direction. Thus, by using a welded bonding pattern in a first panel portion and an adhesive bonding pattern in a second panel portion, secure attachment of curved or angled leg elastic members may be achieved without sacrificing breathability and flexibility of the panel material in areas other than those where the leg elastic members are applied.

The first panel portion comprises no elastic members being substantially inclined in relation to the transversal direction T, and can thus suitably be bonded by a welded bond pattern.

In each of the first panel portion and the second panel portion, the respective top and bottom layers forms a top side surface and a bottom side surface, respectively, of the combined panel. Accordingly, in each of the first panel portion and the second panel portion, the combined panel comprises no additional layers of web material but the top and bottom layers. A front or rear body panel having relatively few layers is beneficial in terms of breathability and comfort as well as in view of sustainability and economics.

A top side surface is a surface forming part of the top side of the panel, i.e. forming part of the inside or the outside of the panel, depending on the orientation of the panel in the article. A bottom side surface is a surface forming part of the bottom side of the panel, i.e. forming part of the inside or the outside of the panel, depending on the orientation of the panel in the article.

In each of the first and second panel portions, the first set of elastomeric strands and the optional second set of elastomeric strands, respectively, are attached between the respective top and bottom layer when in a stretched condition. Accordingly, in a relaxed state of the combined panel, the respective top and bottom layers form wrinkles along the sets of elastomeric strands. The combined panel is extendible from the relaxed state to a stretched-out state in which the sets of elastomeric strands are stretched out along the transversal direction T causing the wrinkles of the combined panel to diminish or to completely flatten out.

The first panel portion, the second panel portion, and the connection region are transversal portions of the combined panel, and extend from the first side edge of the combined panel to the second side edge of the combined panel.

For example, the first panel portion, the second panel portion and the connection region may each be a transversal portion or region of the panel. In other words, the first panel portion, the second panel portion, and the connection region may each have a continuous length in the longitudinal direction as seen over the entire width of the combined panel in the transverse direction.

The welded bond pattern of the first panel portion may comprise pairs of adjacent weld bonds for attaching each of the elastomeric strands of the first set using such pairs of adjacent weld bonds.

The welded bonds are preferably arranged such that the elastomeric strands are mechanically trapped between the weld bonds. For example, each of the elastomeric strands of the first set may be mechanically trapped between the adjacent weld bonds of a plurality of pairs of adjacent weld bonds.

Thus, the first panel portion may be free or at least substantially free from adhesive.

Optionally, the adhesive bonds of the second panel portion are applied to one or more out of the second top layer, the second bottom layer, the optional second set of elastomeric strands, and the leg elastic members.

Optionally, in the second panel portion, each leg elastic member crosses at least one elastic strand out of the second set of elastomeric strands.

In some variants, the connection region D may be infinitely narrow as seen along the longitudinal direction, i.e., the connection region may for example be in the form of a transversely extending line connecting the first panel portion and the second panel portion.

In other variants, the connection region may have an extension in the longitudinal direction of the article, for example the connection region may comprise a joint joining the first panel portion and the second panel portion.

Such a joint joining the first panel portion and the second panel portion may for example be an adhesive joint or a welded joint.

Optionally, the connection region has a length in the longitudinal direction of the article being no more than 10 mm.

Optionally, the connection region has a length in the longitudinal direction of the article being no more than 20% of a total length (in the longitudinal direction of the article) of the combined panel. Preferably, the connection region has a length in the longitudinal direction of the article being no more than 10% of a total length (in the longitudinal direction of the article) of the combined panel, such as no more than 7% or no more than 5% of a total length (in the longitudinal direction of the article) of the combined panel.

Optionally, the first panel portion has a length in the longitudinal direction of the article, the length being at least 20% of a total length of the combined panel, such as at least 25%, or at least 30%. For example, the first panel portion may have a first length being less than 65% of a total length of the combined panel in the longitudinal direction of the article. For example, the first panel portion may have a length in the longitudinal direction of the article being from 25% to 65% of the total length of the combined panel.

Optionally, the second panel portion has a length in the longitudinal direction of the article being at least 20%, such as at least 25% or at least 30% of a total length of the at least one of the combined panel in the longitudinal direction of the article. For example, the second panel portion may have a length being less than 70% of the total length of the combined panel. For example, the second panel portion may have a length being from 25% to 70% of the total length of the combined panel.

Optionally, the second bottom layer and the first bottom layer are formed by sheets of identical sheet material. As such, the bottom side of the combined panel may have a uniform appearance.

Optionally, the second bottom layer and the first bottom layer are formed by a single continuous material sheet, extending over the connection region. As such, the bottom side of the combined panel may have a uniform appearance without any visible joints at the connection region. Also, in such variants, the connection region may comprise no more than three layers of sheet material.

The first panel portion and the second panel portion each comprises no more than two layers of sheet material—this being the result of the respective top layer and bottom layer each forming one out of a top side surface and a bottom side surface of the combined panel in the first panel portion and the second panel portion, and that no intervening layer of sheet material is arranged between the top layer and the bottom layer.

It is to be understood that when it is referred herein to “a top layer being bonded to a bottom layer”, this implies that the top layer is directly bonded to the bottom layer, without any intervening layers of sheet material. However, this does not exclude that the elastic strands and/or any leg elastic members could be involved in the bonding.

Thus, optionally, the first panel portion and the second panel portion each comprises no more than two layers of sheet material and the connection region comprises no more than three layers of sheet material.

Further, as will be explained below, using a single continuous material sheet to form the second bottom layer and the first bottom layer may be advantageous in manufacturing.

Also, when the second bottom layer and the first bottom layer are formed by a single continuous material sheet, and the bottom side of the combined panel is the outside of the absorbent article, this means that the outside surface of the absorbent article is formed by the single continuous material sheet of the combined panel, thus providing the article with a uniform outside appearance without any visible joints.

Optionally, the second top layer and the first top layer are formed by separate material sheets.

Optionally, the second top layer and the first top layer are formed by separate sheets of identical sheet material, such as by separate sheets of the same sheet material.

Optionally, in the connection region, a transversal edge portion of the material sheet of the second top layer is arranged so as to overlap a transversal edge portion of the material sheet of the first top layer in the longitudinal direction, preferably the transversal edge portion of the second top layer is arranged on the top side of the transversal edge portion of the first top layer.

As such, the connection region may have a length in the longitudinal direction as proposed above, where the length of the connection region in the longitudinal direction corresponds to the length in the longitudinal direction of the longitudinal overlap between the first top layer and the second top layer.

Optionally, the top direction is directed towards the inside of the article and the bottom direction is directed towards the outside of the article.

Optionally, the adhesive bonds of the second panel portion comprises, in defined regions overlapping the leg elastic members, a first adhesive applied to one of the second top layer and the second bottom layer, and a second adhesive applied to the other one of the second top layer and the second bottom layer, wherein optionally the second adhesive is different from the first adhesive.

Optionally, the first adhesive is applied substantially continuously over the second top layer.

Optionally, the second adhesive is applied only in defined regions of the second bottom layer overlapping the leg elastic members.

Optionally, the first adhesive is more fast consolidating than the second adhesive.

Optionally, the adhesive bonds of the second panel portion comprises adhesive applied to the second set of elastic strands.

Optionally, the combined panel further comprises a waist panel portion comprising the waist edge of the combined panel.

Optionally, the combined panel defines a transversal waist connection region. In this case the first panel portion extends in the longitudinal direction from the connection region to the waist connection region, and the waist panel portion extends in the longitudinal direction from the waist connection region to the waist edge of the combined panel.

Optionally, the waist panel portion comprises a waist bottom layer and a waist top layer and a third set of elastomeric strands being sandwiched between the waist bottom layer and the waist top layer, the elastomeric strands being aligned substantially in parallel with each other and with the transversal direction, and the elastomeric strands being spaced apart from each other in the longitudinal direction. The waist panel portion is bonded by the waist bottom layer being bonded to the waist top layer by a welded bond pattern attaching the third set of elastomeric strands between the waist bottom layer and the waist top layer

The waist bottom layer forms a bottom side surface of the combined panel, and the waist top layer forms a top side surface of the combined panel.

Optionally, a length of the waist panel portion in the longitudinal direction of the article is at least 5%, such as from 5% to 20%, or from 5% to 15% of a total length of the combined panel in the longitudinal direction of the article.

Optionally, the waist bottom layer and the waist top layer may be formed from the same sheet material being folded along the waist edge.

Optionally, the waist bottom layer and the first bottom layer may be formed by a single continuous material sheet, extending over the transversal waist connection region.

In some variants, the second bottom layer, the first bottom layer, and the waist bottom layer may be formed by a single continuous material sheet, extending over the connection region and the waist connection region.

The waist top layer and the first top layer may each be formed by a separate material sheet, wherein the material sheets are arranged with a longitudinal overlap in the waist connection region.

In some variants, a single continuous material sheet may form the first bottom layer, the waist bottom layer and the waist top layer, wherein the waist bottom layer and the waist top layer are separated by a fold in the single continuous material sheet extending along the waist edge.

As used herein, the term “layer” refers to a layer in a laminate material or in a portion of a laminate material, which layer comprises a sheet formed material or a portion of a sheet formed material. A “layer” can be bonded to another layer by means of weld bonds or adhesive bonds as disclosed herein, sandwiching elastic strands or members between the layers.

The sheet formed material may be a sheet of any suitable material, such as of a nonwoven material.

Optionally, at least one, preferably each, of the second bottom layer, the second top layer, the first bottom layer, the first top layer, and optionally the waist bottom layer, and the waist top layer comprises or consists of a sheet of a nonwoven material.

Optionally, the nonwoven material has a basis weight of from 8 to 26 gsm.

Optionally, the nonwoven material contains thermoplastic fibres, preferably the nonwoven material contains at least 50% thermoplastic fibres and more preferably at least 80% thermoplastic fibres.

Optionally, the second set of elastomeric strands and/or the first set of elastomeric strands and/or optionally the third set of elastomeric strands has a linear mass density, dtex, of from 80 to 1200 dtex.

Optionally, the average spacing in the longitudinal direction between the strands in the second set of elastomeric strands of the second panel portion is no less than the average spacing in the longitudinal direction between the strands in the first set of elastomeric strands in the first panel portion.

Optionally, the spacing in the longitudinal direction between the strands in the first set of elastomeric strands of the first panel portion is from 2 to 20 mm. For example, the spacing between the strands in the first set of elastomeric strands may be constant.

Optionally, the spacing in the longitudinal direction between the strands in the second set of elastomeric strands of the second panel portion is from 2 to 35 . mm. For example, the spacing between the strands in the first set of elastomeric strands may be constant.

Optionally, the spacing in the longitudinal direction between the strands in the third set of elastomeric strands of the waist portion is from 2 to 12 mm. For example, the spacing between the strands in the first set of elastomeric strands may be constant.

A set of elastic strands as referred to herein comprises a plurality of elastic strands.

For example, the first and/or the second set of elastic strands may each comprises a plurality of elastic strands, i.e. no less than two strands. For example, the first and/or second set of elastic strands may each comprise no less than three strands, such as no less than four strands or no less than five strands.

For example, the third set of elastic strands (of the waist portion) may comprise a plurality of elastic strands, such as no less than two strands or no less than three strands.

Optionally, the absorbent core is arranged with a central part of the absorbent core being located in the crotch region and with a front end part of the absorbent core overlapping with the front body panel in a front overlap area and with a rear end part of the absorbent core overlapping with the rear body panel in a rear overlap area.

Optionally, the absorbent core is arranged so as to longitudinally overlap a transversal portion of the connection region of the combined panel.

Optionally, the front body panel is a combined panel and the rear body panel is a combined panel. This implies that the article is provided with leg elastic members in both the front body panel and the rear body panel.

Optionally, one of the front body panel and the rear body panel is a combined panel (i.e. comprises leg elastic members), and the other of the front body panel and rear body panel is free from leg elastic members.

Optionally, one of the front body panel and the rear body panel is a combined panel (i.e. comprises leg elastic members), and the other of the front body panel and rear body panel is free from adhesive.

Optionally, said other of the front body panel and the rear body panel comprises a main panel portion comprising a main bottom layer and a main top layer and a set of elastomeric strands being sandwiched between the main bottom layer and the main top layer, the elastomeric strands being aligned substantially in parallel with each other and with the transversal direction, and the elastomeric strands being spaced apart from each other in the longitudinal direction.

The main portion is bonded by the main bottom layer being bonded to the main top layer by a welded bond pattern.

Optionally, the main panel portion extends at least from the leg edges of said other of the front body panel or rear body panel, respectively and in a direction towards the waist edge of said other of the front body panel and rear body panel.

As mentioned in the above, the front body panel and the rear body panel of the article extends from a first side edge to a second side edge. As such, the front body panel and/or the rear body panel may each consist of a piece of a laminate material which extends continuously from the first side edge to the second side edge.

For example, the combined panel may consist of a piece of combined laminate material which is cut from a combined laminate material web, as will be described in the below.

The disclosure further relates to a combined laminate material web, which can be used in the disposable absorbent pant-type article according to the invention and described herein, for which at least one of the front section or the back section is a combined panel.

Thus, there is provided a combined laminate material web for a disposable hygienic article, the combined laminate material web being continuous as seen in a machine direction and having a width in a cross-direction, perpendicular to the machine direction, between two side edges. The combined laminate material web has a top side and a bottom side as seen in a thickness direction perpendicular to the machine direction and to the cross-direction. The combined laminate material web comprises a first web portion and a second web portion, the first web portion and the second web portion being connected by a connection region extending along the machine direction, such that the first web portion extends from the connection region and in the cross-direction towards the first side edge of the combined laminate material web, and the second web portion extends from the connection region and in the cross-direction towards the second side edge of the combined laminate material web.

The first web portion comprises a first bottom layer and a first top layer being superimposed such that, in the first web portion, the first bottom layer forms a bottom side surface and the first top layer forms a top side surface of the combined laminate material web. The first web portion further comprises a first set of elastomeric strands being sandwiched between the first bottom layer and the first top layer, the elastomeric strands being aligned substantially in parallel with each other and with the machine direction, and the elastomeric strands being spaced apart from each other in the cross-direction, and the first web portion is bonded by the first bottom layer being bonded to the first top layer by a welded bond pattern attaching the first set of elastomeric strands between the first bottom layer and the first top layer, and the second web portion comprises a second bottom layer and a second top layer being superimposed such that, in the second web portion, the second bottom layer forms a bottom side surface and the second top layer forms a top side surface of the combined laminate material web. The second web portion further comprises additional elastic members extending at least partially in a direction other than parallel with the machine direction, wherein the additional elastic members are sandwiched between the second top layer and the second bottom layer. The second web portion is bonded by the second bottom layer being bonded to the second top layer by adhesive bonds, for attaching the additional elastic members between the second bottom layer and the second top layer.

Optionally, the second web portion further comprises a second set of elastomeric strands being sandwiched between the second bottom layer and the second top layer, the elastomeric strands being aligned in parallel with each other and substantially with the machine direction, and the elastomeric strands being spaced apart from each other in the cross-direction. In this case, the second web portion is bonded by the second bottom layer being bonded to the second top layer by adhesive bonds, for attaching the additional elastic members and the second set of elastomeric strands between the second bottom layer and the second top layer.

As such, a front body panel or a rear body panel being a combined panel as described herein may be formed by cutting such a combined material web. Thus, it will be understood that to form a combined panel including any of the optional features as set out herein in relation to the article of the first aspect, corresponding features may be applied to the combined laminate material web of the second aspect.

The disclosure further relates to a method for forming a combined laminate material web, which may be used in a disposable absorbent pant-type article according to the invention and described herein, for which at least one of the front section or the back section comprises or constitutes a combined panel. This method includes the steps described above in the method according to the fourth aspect. This method may further comprise any of the optional steps described above.

Thus, there is provided a method for forming a combined laminate material web for a disposable hygienic article, the combined laminate material web being continuous as seen in a machine direction and having a width in a cross-direction perpendicular to the machine direction between a first side edge and a second side edge, the combined laminate material web having a top side and a bottom side as seen in a thickness direction perpendicular to the machine direction and to the cross-direction. The method comprises a welding bonding step and an adhesive bonding step.

The welding bonding step comprises feeding a first bottom layer web, a first top layer web, and a first set of continuous elastic strands in the machine direction, and bonding the first bottom layer web to the first top layer web by a welded bond pattern so as to attach the first set of continuous elastic strands between the first bottom layer web and the first top layer web, the first set of elastomeric strands being aligned in parallel with each other and substantially with the machine direction, and being spaced apart from each other in the cross-direction.

The adhesive bonding step comprise feeding a second top layer web, a second bottom layer web, in the machine direction, and feeding a set of one or more continuous additional elastic members, and bonding the second bottom layer web to the second top layer web by adhesive bonds so as to attach the second set of elastomeric strands and the set of one or more continuous additional elastic members between the second bottom layer web and the second top layer web. The additional elastic members are extending at least partially in a direction not parallel with the machine direction.

Optionally, the adhesive bonding step further comprises feeding a second set of continuous elastic strands in the machine direction, and bonding the second bottom layer web to the second top layer web by adhesive bonds so as to attach the second set of elastomeric strands and the set of one or more continuous additional elastic members between the second bottom layer web and the second top layer web. The second set of elastomeric strands are being aligned substantially in parallel with each other and with the machine direction, and being spaced apart from each other in the cross-direction.

The additional elastic members extending at least partially in a direction not parallel with the machine direction may form the leg elastic members in an article comprising a combined panel obtained from the combined laminate material web.

That the additional elastic members extend at least partially in a direction other than parallel with the machine direction implies that each additional elastic member is at least partially curved or inclined such that it spans over a CD extension as seen in the cross-direction. Each additional elastic member may for example form an angle of at least 10 degrees with the machine direction, over at least a portion of the CD extension, such as over at least 20% of the CD extension of the additional elastic member. For example, each additional elastic member may from an angle of from 10 to 80 degrees with the machine direction, over at least a portion of the CD extension of the additional elastic member, such as over at least 20% of the CD extension of the additional elastic member.

For example, each additional elastic member may span over a CD extension as seen in the longitudinal cross-direction of at least 50 mm.

For example, each additional elastic member may form an angle of at least 10 degrees with at least one strand in the second set of elastomeric strands. Preferably, each additional elastic member may form an angle of at least 10 degrees, such as from 10 to 80 degrees, with a majority of the strands in the second set of the elastomeric strands.

The welding bonding step and the adhesive bonding step are performed so as to form a first web portion, wherein the first bottom layer web forms a bottom side surface and the first top layer web forms a top side surface of the combined laminate material web, and a second web portion, wherein the second bottom layer web forms a bottom side surface and the second top layer web forms a top side surface of the combined laminate material web. The second web portion is connected to the first web portion via a connection region extending along the machine direction.

Herein, “top” and “bottom” does not relate to any specific vertical orientation of the combined laminate material web, only to relative locations as seen along a thickness direction perpendicular to the cross-direction and the machine direction.

The welding bonding step and the adhesive bonding step are performed such that the first and second sets of elastomeric strands are attached between the respective top and bottom layers when in a stretched state in relation to the respective top and bottom layers. For example, the elastomeric strands may be stretched so as to be elongated to a length being at least 2× a relaxed length of the elastomeric strands, such as to a length being at least 3× a relaxed length of the elastomeric strands. The respective top and bottom layers may be stretched only slightly, i.e., to an amount required to perform the feeding operation. As such, the elastomeric strands are attached when in a stretched state in relation to the respective top and bottom layer.

Optionally, the welding bonding step is performed upstream of the adhesive bonding step in the machine direction. As such, it may be avoided that any adhesive, such as non-consolidated adhesive, risks adhering to the welding equipment used for the welding bonding step, and thereby deteriorate its function and/or require cleaning.

Optionally, in the adhesive bonding step, the elastomeric strands of the second set are aligned substantially in parallel with each other and with the machine direction, and the additional elastic members are extending at least partially in a direction not parallel with the machine direction, such that each additional elastic member crosses at least one elastic strand out of the second set of elastomeric strands.

Optionally, the welded bond pattern in the welding bonding step comprises pairs of adjacent weld bonds for attaching each of the first set of elastomeric strands using such pairs of adjacent weld bonds, as disclosed herein.

Optionally, the adhesive bonds of the adhesive bonding step are applied to one or more of the second top layer, the second bottom layer, the second set of elastomeric strands, and the leg elastic members.

Optionally, the connection region has a width in the cross-direction being no more than 10 mm.

Optionally, the connection region has a width in the cross-direction being no more than 20% of a total width of the combined laminate material web, such as no more than 10% of a total width of the combined laminate material web. For example, the connection region may have a width in the cross-direction being no more than 7% or no more than 5% of a total width of the combined laminate material web.

Optionally, the first web portion has a width in the cross-direction being at least 20% of a total width in the cross-direction of the combined laminate material web, such as at least 25%, or at least 30%. For example, the first web portion may have a width in the cross-direction being less than 65% of a total width of the combined laminate material web. For example, the first panel portion may have a width in the cross-direction being from 25% to 65% of the total width of the combined laminate material web.

Optionally, the second web portion has a width in the cross-direction being at least 20% of a total width in the cross-direction of the combined laminate material web, such as at least 25%, or at least 30%. For example, the second web portion may have a width in the cross-direction being less than 70% of a total width of the combined laminate material web. For example, the second panel portion may have a width in the cross-direction being from 25% to 70% of the total width of the combined laminate material web.

Optionally, the welding bonding step comprises
feeding a combined bottom layer web material to form the first bottom layer web, and
bonding the first top layer web to a first width portion of the combined bottom layer web material so as to form the first laminate web portion, leaving at least a second width portion of the combined bottom layer web material non-laminated, and
the adhesive bonding step comprises
feeding the combined bottom layer web material to form the second bottom layer web, and
bonding the second top layer web to the non-laminated second width portion of the combined bottom web so as to form the second laminate web portion.

Optionally, the second top layer web and the first top layer web are fed as separate web materials.

Optionally, the second top layer web and the first top layer web are formed by webs of the same material.

Optionally, the method comprises comprising feeding a base top layer web in the machine direction, and slitting the base top layer web along the machine direction so as to form the second top layer web and the first top layer web as separate webs from the base top layer web.

Optionally, the adhesive bonding step comprises bonding the second top layer web in a partially overlapping relationship with the first top layer web, as seen along the cross-direction, such that the connection region comprises the overlapping portions of the second top layer web and the first top layer web.

As set out in the above, the method may involve feeding a combined bottom layer web material to form the first bottom layer web and the second bottom layer web, such that the combined bottom layer web material extends over the connection region. Thus, the connection region may be formed so as to comprise no more than three layers of sheet material.

Thus, optionally, the first panel portion and the second panel portion are formed to comprise no more than two layers of sheet material (the respective top and bottom layers) and the connection region is formed so as to comprise no more than three layers of sheet material.

When the method involves feeding a combined bottom layer web material to form the first bottom layer web and the second bottom layer web, such that the combined bottom layer web material extends over the connection region, the first top layer web and the second top layer web could optionally be applied so as to be spaced in the cross-direction on the combined bottom layer web. In such a case, the connection region would comprise only one layer of sheet material, namely the combined bottom layer web material.

In another option, the first top layer web and the second top layer web could be applied edge-to-edge as seen in the cross-direction on the combined bottom layer web. In such a case, the connection region would comprise only the line where the first top layer web and the second top layer web meet. In this case, the number of layers in the connection region will be no more than two.

Optionally, the adhesive bonding step comprises, in defined regions overlapping the leg elastic members, applying a second adhesive applied to one of the second top layer and the second bottom layer, and a first adhesive applied to the bottom one of the second top layer and the second bottom layer, wherein optionally the second adhesive is different from the first adhesive.

Optionally, the second adhesive is applied substantially continuously over the second bottom layer.

Optionally, the first adhesive is applied only in defined regions of the second top layer overlapping the additional elastic members.

Optionally, the second adhesive is more fast consolidating than the first adhesive.

Optionally, the adhesive bonding step comprises applying adhesive to the second set of elastic strands.

Optionally, at least one, preferably each, of the second bottom layer web, the second top layer web, the first bottom layer web, the first top layer web, and optionally the waist bottom layer web, and the waist top layer web comprises or consists of a sheet of a nonwoven material.

Optionally, the nonwoven material has a basis weight of from 8 to 26 gsm.

Optionally, the nonwoven material contains or consists of thermoplastic fibres, preferably the nonwoven material contains contain at least 50% thermoplastic fibres and more preferably at least 80% thermoplastic fibres.

Optionally, the second set of elastomeric strands and/or the first set of elastomeric strands and/or optionally the third set of elastomeric strands has a linear mass density, dtex, of from 80 to 1200 dtex.

Optionally, the average longitudinal spacing between the strands in the second set of elastomeric strands is no less than the average longitudinal spacing between the strands in the first set of elastomeric strands.

Optionally, the longitudinal spacing between the strands in the first set of elastomeric strands is from 2 to 20 mm.

Optionally, the longitudinal spacing between the strands in the second set of elastomeric strands is from 2 to 35 mm.

Optionally, the method further comprises a waist forming step comprising forming a waist laminate web portion of the combined laminate material web, the waist laminate web portion comprising a side edge of the combined laminate material web.

Optionally, the waist laminate web portion is connected to the first laminate web portion via a waist connection region.

Optionally, the waist forming step comprises feeding a waist bottom layer web, a waist top layer web, and a third set of elastomeric strands in the machine direction, and bonding the top waist layer web to the bottom waist layer web by a welded bond pattern attaching the third set of elastomeric strands between the waist bottom layer web and the waist top layer web, wherein the elastomeric strands are aligned substantially in parallel with each other and with the machine direction, and the elastomeric strands are spaced apart from each other in the cross-direction.

Optionally, the waist forming step is performed upstream of the adhesive bonding step, and preferably downstream of the welding bonding step.

Optionally, in the waist laminate web portion, the waist bottom layer web forms a bottom side surface and the waist top layer web forms a top side surface of the combined laminate material web.

Optionally, the waist laminate web portion has a width in the cross-direction being at least 5%, such as from 5% to 10% or from 5% to 10% of the width of the total width of the combined laminate material web.

Optionally, the combined laminate material web is for forming a combined panel being a front body panel or a rear body panel of a disposable pant-like article, wherein the additional elastic members are applied so as to form leg elastic members in the front body panel or rear body panel.

Optionally, the method may further comprise cutting or deactivating the additional elastic members so as to form sets of elastic members separated by a non-elastic portion, where the non-elastic portion is intended to connect to the crotch section of the absorbent article. The sets of additional elastic members thus formed may in turn be cut when forming front or rear body panels out of the continuous material web, such that each set forms one member of a pair of leg elastic members on a first panel, and one member of a pair of leg elastic members on a second panel.

The present disclosure also encompasses a combined laminate material web produced by the method, in particular the combined laminate material web described herein.

The present disclosure also relates to a method for producing a disposable pant-type article comprising providing the combined laminate material web described herein.

Thus, there is provided a method for producing a disposable pant-type article comprising providing a combined laminate material web in accordance with the method of the third aspect, and forming a disposable pant-type article having a front body panel and a rear body panel, a crotch region being located between the front body panel and the rear body panel;
an absorbent assembly comprising an absorbent core being arranged with a central part of the absorbent core being located in the crotch region a second front panel side edge being jointed to a second rear panel side edge in a second side seam; and a first front panel side edge being joined to a first rear panel side edge in a first side seam, wherein at least one out of the front body panel and rear body panel is formed from the combined laminate material web.

Optionally, the method for producing a disposable pant-type article comprises providing a rear body panel web and a front body panel web, at least one out of the rear body panel web and the front body panel web being a combined laminate material web produced in accordance with the third aspect described in the above, and intermittently applying absorbent assemblies to the rear body panel web and the front body panel web, the absorbent assemblies being applied spaced-apart in the machine direction and being attached with a first end part of the core assembly overlapping the rear body panel web and a second end part of the core assembly overlapping the front body panel web,
so as to form a composite web of interconnected precursor articles each precursor article comprising an absorbent assembly. The composite web of precursor articles has a front waist edge and a rear waist edge. The method comprises folding the composite web of interconnected precursor articles along a fold line extending in the machine direction and bringing the rear waist edge of the rear body panel web into alignment with the front waist edge of the front body panel web, forming first and second side seams in each precursor article, the side seams joining the front body panel web with the rear body panel web and being formed in the cross-direction along dividing lines between the precursor articles; and severing individual pant-type articles from the composite web of precursor articles.

Optionally, the top side of the combined laminate web is towards the inside of the article and the bottom side of the combined laminate web is towards the outside of the article.

Optionally, the method comprises arranging the absorbent core with a central part of the absorbent assembly being located in the crotch region and with a front end part of the absorbent core overlapping with the front body panel web in a front overlap area and with a rear end part of the absorbent core overlapping with the rear body panel web in a rear overlap area.

Optionally, the method comprises arranging the absorbent assembly so as to overlap at least a part of the first web portion.

Optionally, the front body panel web is formed from a second combined laminate material web and the rear body panel web is formed from a first combined laminate material web, each of the second and first combined laminate material webs being formed by the method according to the third aspect in the above.

Optionally, one of the front body panel and the rear body panel is formed from a combined laminate material web being formed by the method according to the third aspect in the above, and the other of the front body panel and rear body panel is free from leg elastic members forming extending at least partly in a direction other than parallel with the machine direction.

Optionally, one of the front body panel and the rear body panel is formed from a combined laminate material web being formed by the method according to the third aspect in the above, and the other of the front body panel and rear body panel is free from adhesive.

The front body panel may be formed from a continuous front body panel web which extends transversely across the width of the pant-type article between the second and the first front/rear panel side edges and/or the elastically stretchable rear body panel may be formed from a continuous rear body panel web which extends transversely across the width of the pant-type article between the second and the first rear panel side edges.

In the pant-type disposable articles as disclosed herein, the absorbent core may form part of an absorbent assembly, the absorbent assembly comprising the absorbent core, a liquid permeable topsheet and a liquid impermeable backsheet, wherein the absorbent core is sandwiched between the liquid permeable topsheet and the liquid impermeable backsheet.

The disposable pant-type articles as disclosed herein may comprise an elastic waist feature as described above. The elastic waist feature may form a continuous waistband encircling the waist opening of the disposable pant-type article, or may be applied only to selected portions of the waist opening, such as only along central portions of the waist edge of the front and rear body panels or only along the waist edge of the rear body panel, etc.

The elastic waist feature may be completely overlapping with the front body panel and/or with the rear body panel and may share a waist edge with the corresponding front body panel and/or rear body panel.

The elastic waist feature may be completely overlapping with the front body panel and/or with the rear body panel or may be applied to a waist edge of the front body panel and/or rear body panel and protrude beyond the waist edge of the corresponding front body panel and/or rear body panel.

The elastic waist feature may comprise one or more elastic elements. The waist feature may comprise a plurality of elastic strands or elastic bands. Alternatively, the elastic waist feature may comprise or consist of a band of elastic material, such as elastic nonwoven, elastic foam or elastic film. The elastic waist feature may be attached to the front body panel and/or to the rear body panel as a prefabricated component, such as a waistband component comprising two layers of carrier web with elastic strands or bands attached between the layers of carrier web. An elastic waist feature comprising elastic elements such as strands or bands may alternatively be formed directly on the elastic body panel material by applying the elastic elements to the body panel along a waist edge of the body panel and subsequently covering the elastic elements by a cover web, such as a nonwoven cover web.

Optionally, in some variants, the elastic waist feature may be a component of the disposable pant-type article which is distinct from the body panel material of the front body panel and the rear body panel. In other variants, the elastic waist feature may be an integral waist portion of the front body panel and/or rear body panel of the absorbent article.

### BRIEF DESCRIPTION OF THE DRAWINGS

The absorbent articles and the method for their production as disclosed herein will be further explained hereinafter with reference to the appended drawings wherein:

FIG. 1 shows a perspective view of a variant of a disposable absorbent pant-type article according to the invention;

FIG. 2a shows a planar view of the stretched-out article in FIG. 1 with an absorbent assembly and with open side seams, as seen from an inner surface of the pant-type article;

FIG. 2b shows a schematic sectional view along the longitudinal direction L of a first variant of a back section 4 of the article in FIG. 1:

FIG. 2c shows a schematic sectional view along the longitudinal direction L of a second variant of a combined panel 4 suitable for forming the rear panel 4 of the absorbent pant-type article in FIG. 1;

FIG. 2d shows a schematic sectional view along the longitudinal direction L of a front section 3 of the article in FIG. 1;

FIG. 3 shows a perspective view of another variant of a disposable absorbent pant-type article according to the invention;

FIG. 4 shows a schematic view to illustrate a method according to the invention for forming a laminate web material for at least one of a front section or a back section of a disposable absorbent pant-type article;

FIG. 5 shows a planar view of a variant of a laminate web material according to the invention;

FIG. 6 shows a schematic view to illustrate a method according to the invention for producing a disposable absorbent pant-type article;

FIG. 7a shows a planar view of a second variant of a disposable absorbent pant-type article illustrated in a the stretched-out state with an absorbent assembly and with open side seams, as seen from an inner surface of the pant-type article;

FIG. 7b shows a schematic cross-sectional view along the longitudinal direction L of a variant of a combined panel forming the rear panel of the absorbent pant-type article in FIG. 7a;

FIG. 7c shows a schematic cross-sectional view along the longitudinal direction L of a variant of the front panel of the absorbent pant-type article in FIG. 7a;

FIG. 7d shows a schematic cross-sectional view along the longitudinal direction L of a variant of the combined panel illustrated of FIG. 7a;

FIG. 7e shows a schematic cross-sectional view along the longitudinal direction L of a variant of the combined panel illustrated of FIG. 7a; and

FIG. 7f shows a schematic cross-sectional view along the longitudinal direction L of a variant of the combined panel illustrated of FIG. 7a.

FIGS. 8a-d are schematic views for illustrating a variant of a method for producing a combined laminate material web;

FIG. 9 shows a planar view of a variant of a combined laminate material web;

FIG. 10 shows a schematic view to illustrate a method for producing a disposable pant-type article as shown in FIG. 7a using a combined laminate material web as shown in FIG. 9;

### DETAILED DESCRIPTION

Different aspects of the present disclosure will be described more fully hereinafter with reference to the enclosed drawings. The embodiments disclosed herein can be realized in many different forms and should not be construed as being limited to the aspects set forth herein.

The drawings are schematic and the disposable absorbent pant-type article which shown in the figures is a simplified article. It is to be understood that the article may contain further features, such as barrier cuffs, disposal means, etc. It is also to be understood that the waist elastic disclosed herein is optional or that any other suitable type of waist elastic may be used. The side seams may be reclosable side seams, and the pant-type article may be provided with fastener elements to provide reclosability of the side seams.

The disposable absorbent pant-type may be a pant diaper, a sanitary pant, an incontinence pant, etc.

The method for producing a disposable absorbent pant-type as disclosed herein is described in the context of a disposable absorbent pant-type comprising an absorbent assembly. However, it is to be understood that the method is equally applicable to the production of articles which do not comprise a pre-fabricated absorbent assembly. The method as disclosed herein is useful in the production of any type of disposable pant-type article, such as adult incontinence articles, baby diapers, training pants, swim-pants, disposable underpants, sanitary pants for feminine use, etc.

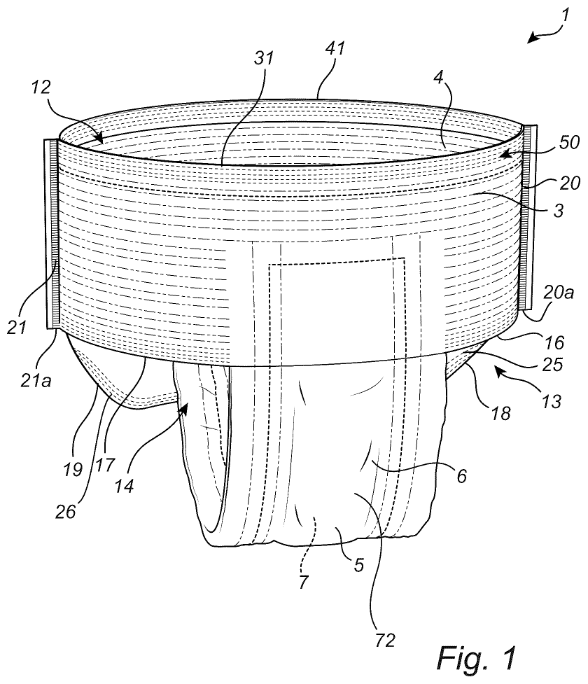

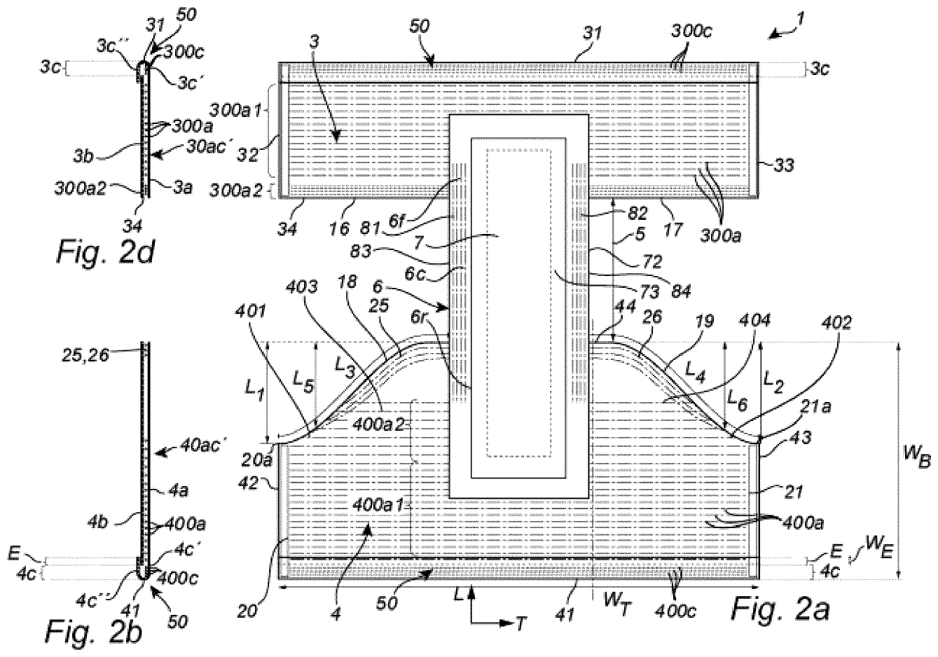

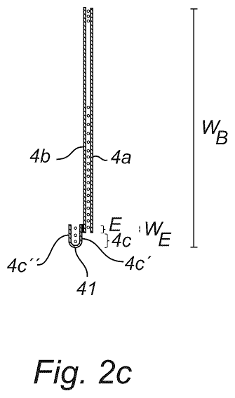

With initial reference to FIGS. 1 and 2a, there is shown a variant of a disposable absorbent pant-type article 1 in the form of a pant-type incontinence article for adult users. The pant-type article 1 is shown in FIG. 1 in an assembled condition ready for use, and in FIG. 2a in an unfolded and flat condition with the side seams 20, 21 opened up. In FIG. 2a, all elastic elements and components are shown in an extended state, such that the pant-type article 1 appears generally two-dimensional. FIG. 2a shows the article from an inner, wearer-facing surface of the article 1.

The pant-type article 1 has a longitudinal direction L and a transversal direction T. Any measurements of distances and dimensions are made with the pant-type article 1 in the fully stretched-out, non-gathered condition, as shown in FIG. 2a. Unless otherwise indicated, the terms “longitudinal” or “longitudinally” as used herein refer to the longitudinal direction L of the pant-type article and the terms “transversal”, “transverse” or “transversely” refer to the transversal direction T of the pant-type article.

The pant-type article 1 comprises a front section 3 which comprises a front section laminate, e.g.

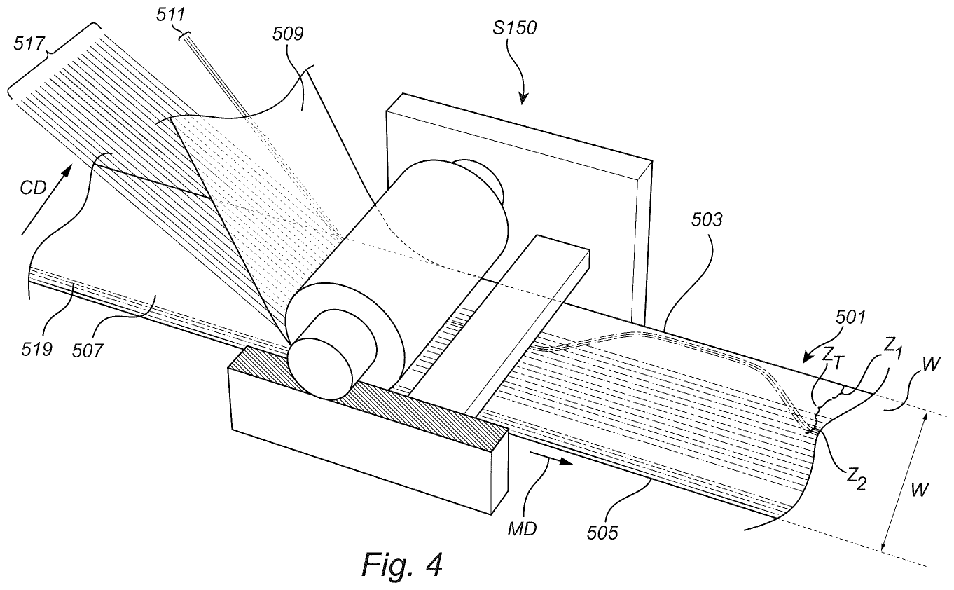

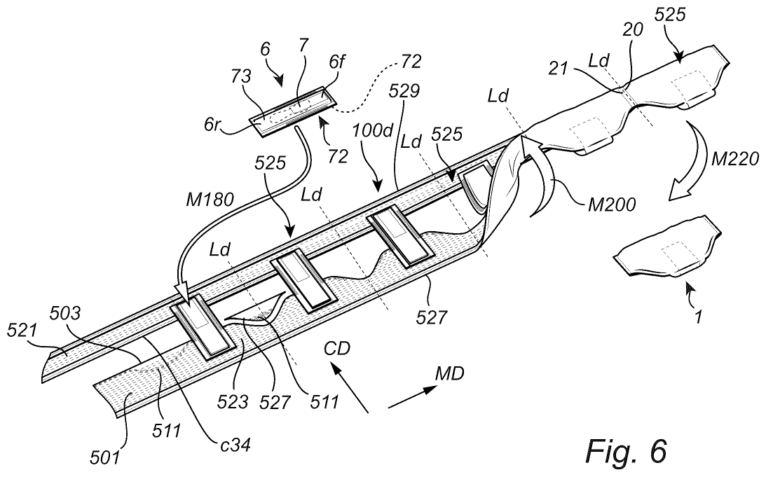

formed from the second laminate web material 521 described in conjunction with FIG. 6. The front section 3 comprises a set of elastic strands 300a which are elastically stretchable in the transversal direction T. The pant-type article 1 further comprises a back section 4, which comprises a back section laminate, e.g. formed from the first laminate web material 501 described in conjunction with FIGS. 4 and 6 below. The back section 4 comprises a set of elastic strands 400a, which are elastically stretchable in the transversal direction T. A crotch region 5 is located between the front section 3 and the back section 4. The elastic strands 300a of the front section 3 provide the overall elastic stretchability of the front section 3. The elastic strands 400a of the back section 4 provide the overall elastic stretchability of the back section 4.

Optionally, as for example illustrated in the variant depicted in FIG. 2a, the set of elastic strands 400a of the back section 4 may be divided into a first subset 400a1 and a second subset 400a2, where the first subset 400a1 differs from the second subset 400a2 as regards longitudinal spacing between the elastic strands, and/or features of the elastic strands, such as diameter or elasticity. For example, the first subset 400a1 may be applied over a majority of the longitudinal extension of the back section 4, whereas the second subset 400a2 may be applied adjacent the leg edges 18, 19 of the back section 4. As such, the second subset 400a2 may be provided to ensure a good fit of the back section 4 at the leg openings 13, 14 of the absorbent article 1. For example, the second subset 400a2 may have a longitudinal spacing between each elastic strand being less than the longitudinal spacing of the first subset 400a1. The second subset 400a2 may comprise a relatively low number of elastic strands, such as from 1 to 8, or from 2 to 6 or from 3 to 5.

Also the set of elastic strands 300a of the front section 3 may be divided into a first subset and a second subset, where the first subset of elastic strands differs from the second subset of elastic strands as regards longitudinal spacing between the elastic strands, and/or features of the elastic strands such as diameter of the elastic strands, although not illustrated in FIGS. 1 and 2a.

The article 1 further comprises an absorbent assembly 6, which is arranged in the crotch section 5 of the pant-type article 1 and which extends in the longitudinal direction L, forwards in over the front section 3 and rearwards in over the back section 4. The absorbent assembly 6 bridges a gap between the front and back sections 3, 4. The absorbent assembly 6 which is shown in FIGS. 1 and 2a is a separately produced component, which comprises an absorbent core 7, a liquid permeable topsheet 73 and a liquid impermeable backsheet 72. The absorbent core 7 is sandwiched between the topsheet 73 and the backsheet 72.

The absorbent assembly 6 is arranged with a central part 6c of the absorbent assembly 6 being located in the crotch section 5, a front end part 6f of the absorbent assembly 6 overlapping with the front section 3 in a front overlap zone and a rear end part 6r of the absorbent assembly 6 overlapping with the back section 4 in a rear overlap zone.

The absorbent core 7 which is shown in the figures has a rectangular shape. However, the disclosure is not limited to rectangular-shaped cores, and it is to be understood that the absorbent core may have any useful shape, such as hourglass shape, T-shape, etc., as known in the art.

The absorbent assembly 6 is applied on the inner surface of the pant-type article 1, the inner surface being the surface which is intended to be facing the wearer when the pant-type article 1 is being worn. The inner surface of the absorbent assembly 6 is formed by the liquid permeable topsheet 73. The absorbent assembly 6 may optionally include barrier cuffs, not shown, extending along the side edges of the absorbent assembly 6.

The topsheet 73 and the backsheet 72 may both extend beyond the periphery of the absorbent core 7 and may be joined to each other in an edge seal extending along the periphery of the absorbent core 7 outward of a peripheral edge of the absorbent core 7. Furthermore, a peripheral edge portion of the absorbent assembly 6 may be formed by only one of the topsheet and the backsheet.

### Absorbent Core

The absorbent core 7 is disposed between the topsheet 73 and the backsheet 72 to absorb liquid, such as urine or other bodily fluids, which has passed through the topsheet 73. The topsheet 73, backsheet 72 and the absorbent core 7 may consist of any materials suitable for their purposes.

An absorbent core 7 as disclosed herein may comprise any conventional material suitable for absorbing discharged bodily wastes, such as cellulosic fluff pulp, tissue layers, highly absorbent polymers super absorbents), absorbent foam materials including hydrogel-foam material, absorbent nonwoven materials, or the like.

The absorbent core 7 may be a single-layer structure or may be a layered structure. The absorbent core 7 may comprise suitable amounts of superabsorbent material. Such superabsorbent material is well known in the field of absorbent articles and is constituted by water-swellable and water-insoluble material which is capable of absorbing large quantities of fluid upon formation of a hydrogel. The absorbent core 7 may contain superabsorbent material in the form of fibers or particles of absorbent polymer material. For example, the superabsorbent material may be surface cross-linked, partially neutralized polyacrylates. The superabsorbent material e.g., the superabsorbent fibers or particles, may be mixed with other absorbent or liquid uptake material or materials, such as cellulose fluff pulp, and/or be arranged in pockets or layers in the absorbent core 7.

The absorbent core 7 may comprise non-absorbent components, such as stiffening elements, shaping elements, binders, etc. Various types of liquid-receiving and liquid distribution elements can also be included in the core.

The absorbent core 7 may further comprise components for improving properties of the absorbent core 7, such as integrity and strength. For example, the absorbent core 7 may comprise a binder or binders, such as binder fibers. Resilient fibers, chemically stiffened fibers, etc. may be present in the absorbent core 7 to counteract wet-collapse of cellulosic fibers. Such fibers may also be useful in retaining a fluid transporting capillary network in the absorbent component so that absorbent fluid may be distributed in the absorbent component and be absorbed by superabsorbent material also in parts of the absorbent component outside the initial wetting area of the absorbent article.

The absorbent core 7 may be a three-dimensionally shaped core and may be provided with features such as embossings, fluid acquisition cavities and fluid distribution channels. Properties of the absorbent core 7, such as density, amount of absorbent material and absorbent capacity, may also be varied in different portions of the absorbent core.

### Topsheet

The materials which are well suited as topsheet materials are soft and non-irritating to the skin, are readily penetrated by body fluids, and display low rewet.

The liquid topsheet 73 is a wearer-facing layer and may comprise or consist of a liquid permeable nonwoven material. Other suitable topsheet materials include tow fibres, porous foams, mesh, apertured plastic films and laminates and combinations of such materials. The topsheet may be made from thermoplastic material, such as thermoplastic synthetic fibers, film or netting. The topsheet 73 may consist of a single layer or may have a laminate structure comprising a plurality of layers, for example, two or more layers. The layers may be made of the same material, or some or all layers may be made of different materials.

### Backsheet

The liquid impermeable backsheet 72 may consist of a thin plastic film, e. g. a polyethylene or polypropylene film, a nonwoven material coated with a liquid impervious material, a hydrophobic nonwoven material which resists liquid penetration or laminates of plastic film and nonwoven. The backsheet material may be breathable to allow vapour to escape from the absorbent body, while still preventing liquids from passing through the backsheet material. The materials which may be used for the backsheet 72 include thin and flexible fluid impermeable plastic films, or fluid impermeable nonwoven materials, fluid impermeable foams and fluid impermeable laminates. The backsheet 72 may be formed by a single layer, or may be a multi-layered structure, with at least one layer being fluid impermeable.

The topsheet and backsheet may be connected to each other for example by adhesive bonding, gluing or welding by heat or ultrasonically. The topsheet and/or the backsheet may further be attached to the absorbent core by any method known in the art, such as adhesive, heat-bonding, welding, needling, etc.

### Front and Back Sections

The front section 3 has a front section waist edge 31 extending in the transversal direction T, a first front section side edge 32 and a second front section side edge 33 extending in the longitudinal direction L and a front section crotch edge 34 extending in the transversal direction T opposite the front section waist edge 31. On each transversal side of the absorbent assembly 6, the front section crotch edge 34 forms a first front leg edge 16 and a second front leg edge 17.

The back section 4 has a back section waist edge 41 extending in the transversal direction T, a first back section side edge 42 and a second back section side edge 43 extending in the longitudinal direction L and a back section crotch edge 44 extending in the transversal direction T opposite the back section waist edge 41. On each transversal side of the absorbent assembly 6, the back section crotch edge 44 forms a first back leg edge 18 and a second back leg edge 19.

In the pant-type article 1 shown in the figures, the front section 3 has been formed from a continuous laminate material web which has been applied in a machine direction MD, corresponding to the transversal direction T of the pant-type article 1, e.g. formed from the second laminate web material 521 described in conjunction with FIG. 6. The front section 3 thereby extends in the transversal direction T across the pant-type article 1 from the first front section side edge 32 to the second front section side edge 33 and extends in the longitudinal direction L from the front waist edge 31 to the front crotch edge 34.

The back section 4 has been formed from a continuous laminate material web which has been applied in a machine direction MD corresponding to the transversal direction T of the pant-type article 1, e.g. formed from the first laminate web material 501 described in conjunction with FIGS. 4 and 6 below. The back section 4 thereby extends in the transversal direction T across the pant-type article 1 from the first back section side edge 42 to the second back section side edge 43 and extends in the longitudinal direction L from the back section waist edge 41 to the back section crotch edge 44.

The crotch section 5 is arranged between the front section 3 and the back section 4 and is contiguous with the front section 3 along the front section crotch edge 34 and is contiguous with the back section 4 along the back section crotch edge 44. The extension of the crotch region 5 in the longitudinal direction L is defined as the distance in the longitudinal direction L between the front section crotch edge 34 and the back section crotch edge 44.

The first front section side edge 33 is intended to be joined to the first back section side edge 43 in a first side seam 21 and the second front section side edge 32 is intended to be joined to the second back section side edge 42 in a second side seam 20, as indicated in FIG. 2a, to form a pant-type article 1 having a waist opening 12, a second leg opening 13 and a first leg opening 14, as shown in FIG. 1.

The waist opening 12 is defined by the front section waist edge 31 and the back section waist edge 41.

The front section 3 forms a pair of leg edges 16, 17, each leg edge extending transversally on a left or right side, respectively, of the absorbent assembly 6. Similarly, the back section 4 forms a pair of leg edges 18, 19, each leg edge extending transversally on a left or right side, respectively, of the absorbent assembly 6. Each of the second leg opening 13 and the first leg opening 14 may be defined between a respective left or right leg edge 16, 17 of the front section 3, of a left or right leg edge 18, 19 of the back section 4, and of a corresponding first and second longitudinal side 83, 84 of the absorbent assembly 6.

In the illustrated embodiment of FIGS. 1 and 2a, the leg edges 16, 17 of the front section 3 extends in the transversal direction T only, but it is also feasible that they extend both in the transversal direction T and in the longitudinal direction L. Further, in the illustrated embodiment of FIGS. 1 and 2a, the leg edges 18, 19 of the back section 4 extend both in the transversal direction T and in the longitudinal direction L, but it is also feasible that they extend in the transversal direction T only. However, in a disposable absorbent pant-type article according to the invention, at least one of the front section 3 and the back section 4 has leg edges that extend both in the transversal direction T and in the longitudinal direction L.

### Leg Elastic Members

At least one out of the front section 3 and the back section 4 comprises a pair of leg elastic members arranged adjacent to the corresponding pair of leg edges.

For example, and as exemplified in the variant of FIGS. 1 and 2a, the back section 4 of the absorbent article 1 comprises a pair of leg elastic members 25, 26 arranged adjacent to the pair of leg edges 18, 19 of the back section 4. The leg elastic members 25, 26 are arranged along part of the leg edges 18, 19 of the back section 4. The leg edges 18, 19 are curved so as to form leg openings comfortably following the legs of wearer. The leg edges 18, 19 being curved implies that the leg edges 18, 19 extend in the transversal direction T and in the longitudinal direction L.

Each leg elastic member 25, 26 may comprise individual elastic elements, such as 2 to 15 individual elastic elements, 3 to 12 individual elastic element or 4 to 10 individual elastic elements. The individual elastic elements may be elastic/elastomeric strings, threads, strands or bands which are applied spaced apart along the rear leg edge sections of the second and first leg edges.

The leg elastic members 25, 26 each extends at least partially in a direction other than parallel with the transversal direction T. Accordingly, the leg elastic members 25, 26 follow the contour of the leg opening 13, 14 so as to allow for an improved fit of the absorbent article around the leg of the wearer.

For example, at least a part of each leg elastic member 25, 26 may form an angle of at least 20 degrees with the transversal direction T, such as at least 30 degrees with the transversal direction T. For example, at least a part of each leg elastic member 25, 26 may form an angle of from 20 degrees to 60 degrees with the transversal direction T, such as from 30 degrees to 45 degrees with the transversal direction T. For example, as in the variant of the figures, at least a part of each leg elastic member 25, 26 forms an angle of about 40 degrees with the transversal direction T.

For example, each leg elastic member 25, 26 may form an angle of at least 20 degrees with the transversal direction T over at least 10% of the total width as seen in the transversal direction T of the back section 4. For example, each leg elastic member may form an angle as set out in the above over at least 10%, such as at least 20% of the total width of the at least one the least one out of the front section 3 and the back section 4.

For example, as may be seen in the example of FIG. 2a, the leg elastic members 25, 26 may substantially follow a curved shape of the leg edges 18, 19.

As exemplified in FIG. 2a and as may be gleaned in FIG. 2b, showing a section in a direction along the longitudinal axis L of the back section 4 in FIG. 2a just outside the absorbent assembly 6, the back section 4 comprises a bottom layer 4a and a top layer 4b being superimposed such that, in the back section 4, the bottom layer 4a forms a bottom side surface and the top layer 4b forms a top side surface of the back section 4. In the product, either the bottom layer 4a or the top layer 4b may form the inner side, i.e., the side against the wearer.

As will be further described in the below, the bottom layer 4a and the top layer 4b are sandwiching elastic strands and/or elastic members to form the elastic back section 4. By virtue of the top layer 4b forming a top side surface and the bottom layer 4a forming a bottom side surface of the back section 4, the back section 4 may be formed to be relatively thin and without using unnecessary amounts of material. That no additional layers of web material are present in the back section 4 may be advantageous in terms of breathability, comfort, sustainability, and economics.

The back section 4 comprises a set of elastic strands 400a being sandwiched between the bottom layer 4a and the top layer 4b. The elastic strands 400a are aligned in parallel with each other and substantially with the transversal direction T, and the elastic strands 400a are spaced apart from each other in the longitudinal direction. As such, the set of elastic strands 400a imparts elasticity in the transversal direction T to the back section 4.

The back section 4 further comprises the leg elastic members 25, 26 arranged adjacent the leg edges 18, 19. The leg elastic members 25, 26 are sandwiched between the top layer 4b and the bottom layer 4a, just as the elastic strands 400a.

The back section 4 may be bonded by adhesive bonds, wherein the adhesive bonds are applied to one or more of the top layer 4b, the bottom layer 4a, the set of elastic strands 400a, and the leg elastic members 25, 26. To this end, prior known adhesive techniques may be used.

In the embodiment illustrated in FIG. 2a, the back section 3 comprises a first leg top elastic strand 401 and a second leg top elastic strand 402 extending in the transversal direction T. The first leg top elastic strand 401 ends at the first leg edge 18 in a position being between the first leg elastic member 25 and the first side seam 20 as seen in the longitudinal direction L of the article 1. The second leg top elastic strand 402 ends at the second leg edge 19 in a position being between the second leg elastic member 26 and the second side seam 21 as seen in the longitudinal direction L of the article 1. Hence, the first and second top elastic strands 401, 402 are able to provide elasticity in a respective zone of the respective leg edge 18, 19 being located between the end of the leg elastic member 25, 26 and the side seam 20, 21.

In the absorbent article 1, the first and second leg top elastic strands 401, 402 are formed by two separated elastic strands, with a gap between them, e.g. allowing space for the absorbent core 7. Hence, it is avoided that the absorbent assembly 6 or the absorbent core 7 is wrinkled by the first and second leg top elastic strands 401, 402. However, when the article 1 is manufactured, the first and second leg top elastic strands 401, 402 may be provided as a single continuous elastic strand, as is further described herein.

Further, the back section 4 comprises a number of first leg side elastic strands 403, extending in the transversal direction T, which cross the first leg elastic member 25, and a number of second leg side elastic strands 404, extending in the transversal direction T, which cross the second leg elastic member 26. Hence, the leg side elastic strands 403, 404 end at the respective leg edges 18, 19 further away from the side seam 20, 21 as seen in the longitudinal direction L than the leg elastic member 25, 26.

The first and second leg side elastic strands 403, 404 are formed by two separated elastic strands, with a gap between them, e.g. allowing space for the absorbent core 7. Hence, it is avoided that the absorbent core 7 is wrinkled by the first and second leg side elastic strands 403, 404. However, when the article 1 is manufactured, the first and second leg side elastic strands 403, 404 may be provided as continuous elastic strands, as is further described herein.

In the illustrated embodiment, the first and second leg top elastic strands 401, 402 and the first and second leg side elastic strands 403, 404 form part of the set of elastic strands 400a extending substantially or completely in the transversal direction T of the article 1. As mentioned above, the individual elastic elements may be located at equal distances from each other or may have varying interspaces. Further, they are typically provided in a stretched state when manufacturing the article.

The first leg edge 18 has a longitudinal extension L₁ as seen in the longitudinal direction L of the article 1. The first leg elastic member 25 ends at a longitudinal position of the first leg edge 18 being located within a range of from 1% to 60%, preferably within a range of from 10% to 50%, more preferably within a range of from 20% to 40%, of the longitudinal extension L1 as determined from a lower end 20a of the first side seam 20. Thereby, there is an interspace between the first side seam 20 and the first leg elastic member 25.

The second leg edge 19 has a longitudinal extension L₂ as seen in the longitudinal direction L of the article 1, typically the same as the longitudinal extension L₁ of the first leg edge 18. The second leg elastic member 26 ends at a longitudinal position of the second leg edge being located within a range of from 1% to 60%, preferably within a range of from 10% to 50%, more preferably within a range of from 20% to 40%, of the longitudinal extension L₂, as determined from a lower end 21a of the second side seam 21. Thereby, there is an interspace between the second side seam 21 and the second leg elastic member 26, typically the same as for the first leg edge 18.

If instead expressed in millimetres, the first leg elastic member 25 ends at a longitudinal position of the first leg edge 18 being located within a range of from 1 mm to 80 mm, preferably within a range of from 10 mm to 70 mm, more preferably within a range of from 20 mm to 55 mm as determined from the lower end 20a of the first side seam 20. The second leg edge 19 ends at a longitudinal position of the second leg edge 26 being located within a range from 1 mm to 80 mm, preferably within a range of from 10 mm to 70 mm, more preferably within a range of from 20 mm to 55 mm.

Hence, the first leg elastic member 25 extends along a distance being within a range 50%-99% of a length L₃ of the first leg edge 18, preferably 55%-95%, more preferably 60%-90%, and the second leg elastic member 26 extends along a distance being within a range 50%-99% of the length L₄ of the second leg edge 19, preferably 55%-95%, more preferably 60%-90%. Thereby, the lengths L₃, L₄ of the respective leg edge 18, 19 are measured along the leg edge 18, 19 itself following its curvature and thus extend both in the longitudinal L and the transversal T directions.

Further, the first leg elastic member 25 extends a distance being within a range of 150-300 mm along the first leg edge 18, preferably 165-285 mm, more preferably 180-270 mm and the second leg elastic member 26 extends a distance being within a range of 150-300 mm along the second leg edge 19, preferably 165-285 mm, more preferably 180-270 mm.

The first leg elastic member 25 extends along a longitudinal length L₅ being less than 40% preferably less than 35%, more preferably less than 30% of a total longitudinal length WB of the back section 4, and the second leg elastic member 26 extends along a longitudinal length L₆ being less than 40% preferably less than 35%, more preferably less than 30% of the total longitudinal length W_{B} of theack section 4.

In the illustrated embodiment, the first leg elastic member 25 extends from the absorbent assembly 6 to the first leg edge 18 and the second leg elastic member 26 extends from the absorbent assembly 6 to the second leg edge 19. The first and second leg elastic members 18, 19 may also extend below at least part of the absorbent assembly 6, but preferably no leg elastic member extends continuously under the whole width of the absorbent assembly 6. Thereby, the risk of wrinkling the absorbent assembly 6, and in particular the absorbent core 7 by means of leg elastic members 25, 26 going over the whole width of the absorbent assembly 6 and 7 or the absorbent core 7, is avoided. This may be achieved by deliberately interrupting the leg elastic member in a central area of the article, e.g. by cutting it, such that a gap is provided for the absorbent core. Methods for accomplishing this are described in in WO 2018/106160 A1, to which we refer.

The article 1, as an option, further comprises a first and a second crotch elastic member 81, 82, extending along a first and a second longitudinal side 83, 84 of the absorbent assembly 6. Thereby, the crotch elastic members 81, 82 form part of the elasticity around the leg openings 13, 14. The crotch elastic members 81, 82 may comprise one or more elastic elements, e.g. in the form of a string, thread, strand or band, illustrated as four elastic strands.

The disposable absorbent pant-type article 1 as disclosed herein may have an elastic waist feature 50 arranged along the waist opening 12, as illustrated i.a. in FIG. 1 and FIG. 2a. An elastic waist feature 50 may include one or more elastic elements extending parallel with the front section waist edge 31 and/or the back section waist edge 41. The elastic waist element or elements may be incorporated in the front section 3 and/or back section 4 or may be applied as a separate waistband, which is attached to the front section waist edge 31 and the back section waist edge 41, or, which is attached to the front section 3 and/or to the back section 4 with the waistband completely overlapping with the corresponding front section 3 and/or back section 4 and sharing a common waist edge. The disposable absorbent pant-type article 1, which is shown in FIGS. 1-6 has an elastic waist feature 50 which extends around the full circumference of the waist opening 12. However, an elastic waist feature may be arranged only along a part of the waist opening, such as only along the back section waist edge, only along the front section waist edge or along a part of one or both of the front section waist edge and the back section waist edge, which part has a length which is less than the full length of the corresponding front section 3 and/or the back section waist edge.

For example, to form an elastic waist feature 50, the back section 4 may comprise a waist panel portion 4c as may be gleaned in FIG. 2b.

In the waist panel portion 4c, a waist bottom layer 4c′ and a waist top layer 4c″ are superimposed such that the waist bottom layer 4c′ forms a bottom side surface and the waist top layer 4c″ forms a top side surface of the back section 4.

The waist panel portion 4c further comprises a set of elastomeric waist strands 400c being sandwiched between the waist bottom layer 4c′ and the waist top layer 4c″, the elastomeric waist strands 400c being aligned substantially in parallel with each other and with the transversal direction T, the elastomeric waist strands 400c being spaced apart from each other in the longitudinal direction L, however typically with less interspace than the set of elastic strands 400a, or even being located next to each other.

The waist panel portion 4c is joined to the back section 4 by a waist connection region E. The waist panel portion 4c extends from the waist connection region E to the waist edge 41 of the back section 4. It is preferred that the waist connection region E has a relatively short longitudinal length W_{E}, for example no more than 10% or no more than 5% of the longitudinal length W_{B} of the back section 4.

Numerous options for forming a waist panel portion 4c on a front section 3 and/or on a back section 4 are known and may be combined with the present disclosure. For example, the different variants described in WO 2018/09771 A1 may be used on the front and/or back sections as described herein. For example, the different variants as described in EP 3 788 997 A1 may be used on the front and/or back sections as described herein.

Since the waist panel portion 4c is free from elastic members extending in directions other than in or substantially parallel to the transversal direction T, the waist panel portion 4c may be bonded a by welded bond pattern bonding the waist bottom layer 4c′ to the waist top layer 4c″ so as to attach the set of elastic waist strands 400c therebetween.

In some variants, see e.g. FIG. 2b, the waist bottom layer 4c′, the waist top layer 4c″ and the bottom layer 4a may be formed by a single continuous material sheet 40ac′. As such, a uniform appearance of the bottom side of the front and/or back section 3, 4 may be achieved, being particularly desired when the bottom direction is the outward direction of the article 1. Moreover, advantages may be obtained in manufacturing as will be described in the below. In order to form the waist top layer 4c″ from the continuous material sheet 40ac′, the sheet 40ac′ is folded along a folded edge thus forming the waist edge 41.

In the variant of FIG. 2c, the waist bottom layer 4c′ and the waist top layer 4c″ may be formed from a continuous material sheet being folded upon itself along the waist edge 41. In this variant, the material sheets of the top layer 4b and the waist bottom layer 4c′ overlap in the longitudinal direction L in the waist connection region E.

Generally, the elastic waist feature 50 may be made in any suitable manner known in the art. For example, the waist connection region E may comprise a joint. The waist bottom layer 4c′ and the waist top layer 4c″ may be arranged so as to overlap with the bottom layer 4a and/or the top layer 4b in the waist connection region E.

Although, in the illustrated pant-type article 1, only the back section 4 comprises leg elastic members 25, 26, also the front section 3 may comprise leg elastic members.

In the variant of FIG. 1, the front section 3 of the absorbent article is free from leg elastic members. As seen in FIG. 2d, showing a section in the longitudinal direction L of the front section 3 in FIG. 2a, the front section 3 comprises a bottom layer 3a and a top layer 3b and a set of elastic strands 300a being sandwiched between the bottom layer 3a and the top layer 3b, the elastic strands 300a being aligned in parallel with each other and substantially with the transversal direction T, and the elastic strands 300a being spaced apart from each other in the longitudinal direction L.

The bottom layer 3a is bonded to the top layer 3b, thereby attaching the set of elastic strands 300a. The bottom layer 3a forms a bottom side surface and the top layer 3b forms a top side surface of the front section 3.

The front section 3 may be provided with a waist feature 50, similar to what has been described in the above for the back section 4. In the variant of FIG. 2d, the front section 3 comprises a waist panel portion 3c comprising a top waist panel layer 3c″ and a bottom waist panel layer 3c′, where the bottom waist panel layer 3c′ is formed by a combined bottom panel sheet 30ac′, which also forms the bottom layer 3a, similar to the waist feature 50 as described in relation to FIG. 2b. The waist panel portion 3c of the front section 3 may be bonded by a welded bond pattern. The waist feature 50 comprises a set of elastomeric waist strands 300c being sandwiched between the waist bottom layer 3c′ and the waist top layer 3c″.

The front section 3 being free from leg elastic members 25, 26 extending in a direction other than parallel with the transversal direction T implies that the front section 3 as a whole may suitably be bonded by a welded bond pattern. Thus, the front section 3 may be free from adhesive.

In some variants, the set of elastic strands 300a of the front section 3 may be divided into a first subset 300a1 and a second subset 300a2, where the first subset 300a1 differs from the second subset 300a2 as regards longitudinal spacing between the elastic strands, and/or features of the elastic strands such as diameter of the elastic strands. For example, the first subset 300a1 may be applied over a majority of the longitudinal extension of the front section 3, whereas the second subset 300a2 is applied adjacent the leg edges 16, 17 of the front section 3. As such, the second subset 300a2 may be provided to ensure a good fit of the front section 3 at the leg openings 13, 14 of the absorbent article 1. For example, the second subset 300a2 may have a longitudinal spacing between each strand being less than the longitudinal spacing of the first subset 300a1. The second subset 300a2 may comprise a relatively low number of elastic strands, such as from 1 to 5 strands, or, from 2 to 4 strands.

In other variants, the front section 3 may be provided with a leg panel portion which may be formed in the same manner as previously described for the waist panel portion, to ensure a good fit of the front section 3 at the leg openings 13, 14 of the absorbent article. Thus, a leg panel portion may be connected to the front section via a leg connection portion, similar to the waist connection portion described in the above.

In the embodiment shown in FIGS. 1 and 2a, the front section 3 and the back section 4 are formed by two separate panels, i.e. a front body panel and a rear body panel, respectively. The crotch section 5 is formed by a portion of the absorbent assembly 6, such that this portion of the absorbent assembly 6 spans the gap between the front section 3 and the back section 4.

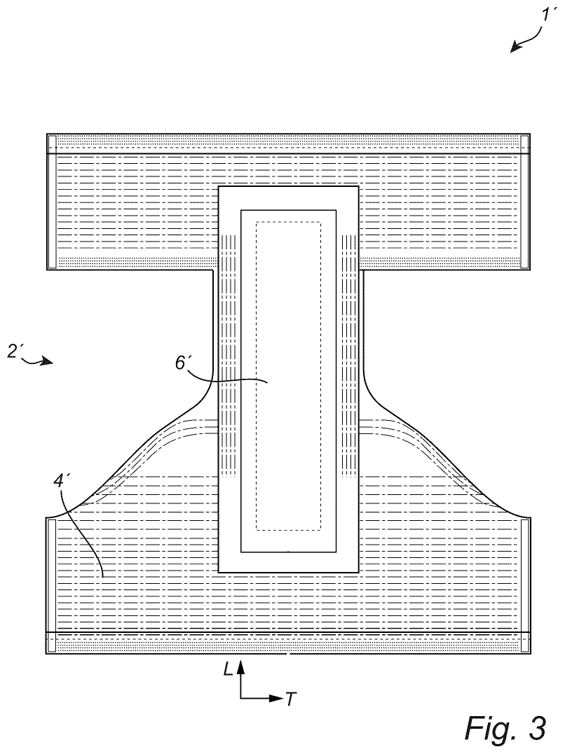

In an alternative embodiment, illustrated in FIG. 3, the front section 3′, the back section 4′ and the crotch section 5′ are made of a chassis 2′ forming a single piece. The absorbent assembly 6′ is located at a central part of the chassis 2′. This embodiment may have the same features and optional features as described herein for the embodiment shown in FIGS. 1 and 2a.

### Forming a Laminate Web Material

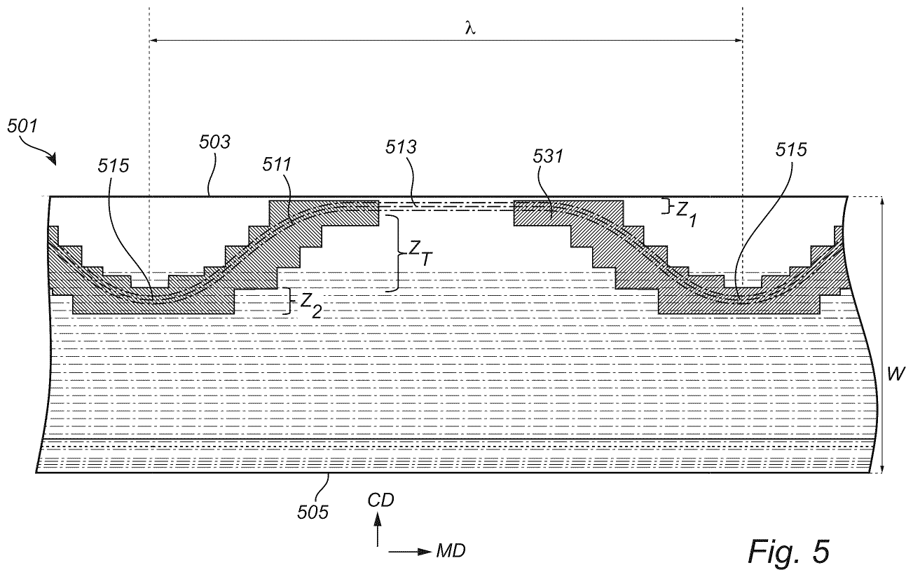

FIG. 4 schematically illustrate a method according to the invention for forming a laminate web material 501. FIG. 5 illustrates a section of the laminate web material 501. The laminate web material 501 is continuous as seen in a machine direction MD and has a width W in a cross direction CD between a first side edge 503 and a second side edge 505.

The laminate web material 501 may be used as a laminate web material for at least one of a front section 3 or a back section 4 of a disposable absorbent pant-type article 1 as described herein, see e.g. FIG. 1. Hence, the laminate web material 501 may be seen as an intermediate product when manufacturing the disposable absorbent pant-type article 1. Details and advantages described herein for the article 1 or its manufacture, are, as far as they are applicable, valid also for the laminate web material 501.

The method comprises:
S110: Feeding a bottom layer web 507 in the machine direction MD.
S120: Feeding a top layer web 509 in the machine direction MD.
S125: Feeding a set 517 of continuous elastic strands in the machine direction MD.
S130: Feeding a continuous leg elastic member 511 comprising one or more elastic elements.
S 140: Positioning the continuous leg elastic member 511, such that it extends over less than 40% of the width W of the laminate web material 501, preferably less than 35%, more preferably less than 30%.
S150: Bonding the bottom layer web 507 to the top layer web 509 to form the laminate web material 501, thereby sandwiching the set 517 of continuous elastic strands between the bottom layer web 507 and the top layer web 509, such that the elastic strands of the set 517 of continuous elastic strands are aligned substantially in parallel with each other and with the machine direction MD, and being spaced apart from each other in the cross direction CD, and sandwiching the continuous leg elastic member 511 between the bottom layer web 507 and the top layer web 509.

The terms bottom layer web and the top layer web as used herein refer to the way that they are typically provided in the manufacturing equipment, exemplified in FIG. 4. In the disposable absorbent pant-type article 1, the bottom layer web 507 may either form the inner side, i.e., the side facing the wearer, or the outer side of the article 1. The bottom layer web 507 and the top layer web 509 are supplied as continuous in the machine direction.

The first edge 503 of the laminate web material is the edge from where the leg cuts will be made. Hence, the continuous leg elastic member 511 partly extends along and adjacent to this edge 503 in the first zone Z₁. The continuous leg elastic member 511 is applied in an undulating way as seen in the cross direction CD. One flat apex 513 of the undulating wave is thus in the first zone Z₁, while the other flat apex 515 of the undulating wave is in the second zone Z₂. In the first and second zones Z₁, Z₂, the continuous leg elastic member 511 extends parallel with or substantially parallel with the machine direction MD. The wavelength A of the undulating wave corresponds to the width W_{T} of the disposable absorbent pant-type article 1 as taken in transverse direction T, cf. FIG. 2a. In between the first and second zones Z₁, Z₂, there is a transfer zone Z_{T}, in which the continuous leg elastic member 511 extends both in the cross direction CD and the machine direction MD. Further details of the undulation can be derived from the description herein of the first and second leg elastic members 25, 26 in the disposable absorbent pant-type article 1. Later on in manufacturing, when absorbent assemblies 6 are applied to the laminate web material 501, the absorbent assemblies 6 are preferably positioned such that they overlap with the continuous leg elastic member 511 in the first zone Z₁. Furthermore, the leg cuts will preferably extend through the transfer zone Z_{T} and optionally into the second zone Z₂.

The continuous leg elastic member 511 extends over less than 40% of the width W of the laminate web material 501, preferably less than 35%, more preferably less than 30%. This facilitates manufacturing a disposable absorbent pant-type article 1, in which the first leg elastic member 25 ends at or in the vicinity of the first leg edge 18, and the second leg elastic member 26 ends at or in the vicinity of the second leg edge 19, as can be seen in FIG. 1.

FIG. 4 further illustrates:
S125: Feeding a set of continuous elastic strands 517 in the machine direction MD, wherein step S150 further comprises attaching the set of continuous elastic strands 517 between the bottom layer web 507 and the top layer web 509, thereby sandwiching the set of continuous elastic strands 517 between the bottom layer web 507 and the top layer web 509, such that the elastic strands of the set of elastic strands 507 are aligned substantially in parallel with each other and with the machine direction MD and being spaced apart from each other in the cross direction CD.

Step S140 further comprises positioning the continuous leg elastic member 511, such that it extends for preferably at least 10 mm in a direction parallel with or substantially parallel with the machine direction MD in a first zone Z₁ along and adjacent to the first edge 503 of the laminate web material 501, extends for preferably at least 10 mm in a direction parallel with or substantially parallel with the machine direction MD in a second zone Z₂ spaced apart in the machine direction MD and the cross direction CD from the first zone Z₁, and extends both in the cross direction CD and the machine direction MD in a transfer zone Z_{T} located between the first and second zones Z₁, Z₂.

The continuous leg elastic member 511 is positioned, such that it crosses at least one elastic strand of the set of continuous elastic strands 517. The crossed elastic strand may then form a leg side elastic strand 403, 404, as seen in FIG. 2a.

The strands of the set of continuous elastic strands 517 are aligned in parallel with each other and substantially with the machine direction MD. Further, FIG. 4 shows how the continuous leg elastic member 511 is fed along the machine direction MD. The continuous leg elastic member 511 is to be positioned such that it extends at least partially in a direction not parallel with the machine direction MD. For example, and as illustrated in FIG. 4, the continuous leg elastic member 511 may be arranged in an undulating pattern suitable for forming leg elastic members 25, 26 in a front section 3 or a back section 4 of a disposable absorbent pant-type article 1.

FIG. 4 also schematically illustrates a bonding step S150. The strands of the set of continuous elastic strands 517 and the continuous leg elastic member 511 are sandwiched between the bottom layer web 507 and the top layer web 509, and adhesive bonds are applied so as to form the laminate web material 501 in the bonding step S150. The adhesive bonds may be applied to one or more of the bottom layer web 507, the top layer web 509, the strands of the set of continuous elastic strands 517, and continuous leg elastic member 511.

For example, and as described herein in relation to FIG. 5, a first adhesive may be applied substantially continuously to the bottom layer web 507. In addition, a second adhesive may be applied in defined regions 531 of the top layer web 509 overlapping the continuous leg elastic member 511 Optionally, the second and first adhesives may be different, for example in that the second adhesive may be more fast curing than the first adhesive. Further, the adhesive bonding step may comprise applying adhesive to the strands of the set of continuous elastic strands 517.

In addition, or as a complement to the adhesive, step S150 may comprise performing welding, e.g. ultrasonic welding and/or thermowelding.

The provision of the waist feature 50 is only schematically illustrated as providing one or more elastic waist elements 519, which will form elastomeric waist strands 400c, e.g. as described herein for the article 1.

The intermediate product, i.e. the laminate web material 501, may be provided with leg cuts in:
S160: Making leg cuts going from the first side edge 503 of the laminate web material 501, thereby cutting through the continuous leg elastic member 511. Step S160 may thereby comprise cutting through the continuous leg elastic member 511 in the second zone Z₂. However, leg cutting is normally done when manufacturing the disposable absorbent pant-type article 1, as described below.

The intermediate product, being the laminate web material 501, is illustrated in FIG. 5. It is continuous as seen in a machine direction MD and has a width in a cross direction CD between the first side edge 503 and the second side edge 505. The laminate web material 501 comprises a bottom layer web 507, a top layer web 509, a continuous leg elastic member 517 sandwiched between the bottom layer web 507 and the top layer web 509, wherein the continuous leg elastic member 517 extends for at least 10 mm in a direction parallel with or substantially parallel with the machine direction in the first zone Z₁ along and adjacent to the first side edge 503 of the laminate web material 501, for at least 10 mm in a direction parallel with or substantially parallel with the machine direction MD in a second zone Z₂ spaced apart from the first zone Z₁, and both in the cross direction CD and the machine direction MD in a transfer zone Z_{T} located between the first and second zones Z₁, Z₂.

The laminate web material 501 further comprises a set of continuous elastic strands 517 in the machine direction MD sandwiched between the bottom layer web 507 and the top layer web 509, the elastic strands of the set of elastic strands 517 being aligned substantially in parallel with each other and with the machine direction MD and being spaced apart from each other in the cross direction CD.

At least one elastic strand of the set of continuous elastic strands 517 crosses the continuous leg elastic member 511 in order to form a leg side elastic strand 403, 404, as seen in FIG. 2a.

The invention further relates to a method, see FIGS. 4-6, for producing a disposable absorbent pant-type article 1, such as a pant diaper, a sanitary pant or incontinence pant, e.g. as illustrated in FIGS. 1-3. The article 1 comprises a front section 3, a back section 4, and a crotch section 5 being located between the front section 3 and the back section 4, wherein an absorbent assembly 6 is arranged with a central part 6c of the absorbent assembly 6 being located in the crotch section 5. The method comprises:
M110: Feeding a bottom layer web 507 in the machine direction MD, see FIG. 4.
M120: Feeding a top layer web 509 in the machine direction MD, see FIG. 4.
M125: Feeding a set of continuous elastic strands in the machine direction,
M130: Feeding a continuous leg elastic member 511 comprising one or more elastomeric elements,, see FIG. 4.
M140: Positioning the continuous leg elastic member 511, such that it extends at least partly in an extension direction being both in the cross direction CD and the machine direction MD, see FIG. 4.
M150: Bonding the bottom layer web 507 to the top layer web 509 to form a first laminate web material 501, thereby sandwiching the set 517 of continuous elastic strands between the bottom layer web 507 and the top layer web 509, such that the elastic strands of the set 517 of continuous elastic strands are aligned substantially in parallel with each other and with the machine direction MD, and being spaced apart from each other in the cross direction CD and sandwiching the continuous leg elastic member 511 between the bottom layer web 507 and the top layer web 509, see FIG. 4.
M160: Positioning the first laminate web material 501 in a position corresponding to one of a front section 3 or a back section 4 of the article 1, see FIG. 6, where it will form the back section 4.
M170: Providing a second laminate web material 521 and positioning the second laminate web material 521 in a position corresponding to the other of the front section 3 or the back section 4 of the article 1, see FIG. 6.
M180: Intermittently providing absorbent assemblies 6 spaced apart in the machine direction MD and positioning them with a first end portion 6r in overlap with the first laminate web material 501 and a second end portion 6f in overlap with the second laminate web material 521, thereby forming a composite web 523 of interconnected precursor articles 525, see FIG. 6.
M190: Making leg cuts 527 going from a first side edge 503 of the first laminate web material 501, thereby cutting through the continuous leg elastic member 511, the leg cuts 527 being positioned in between the absorbent assemblies 6 as seen in a machine direction MD, see FIG. 6.
M200: Folding the composite web 523 along a fold line extending in the machine direction MD, thereby bringing a first edge 527 of the composite web 523 into alignment with a second edge 529 of the composite web 523, see FIG. 6.
M210: Forming first and second side seams 20, 21 in each precursor article 525, thereby joining the first laminate web material 501 with the second laminate web material 521, the side seams 20, 21 being formed in the cross direction CD along dividing lines La between the precursor articles 525, see FIG. 6.
M220: Severing individual disposable absorbent pant-type articles 1 from the composite web 523 of precursor articles 525, see FIG. 6.

The method may be used to manufacture the disposable absorbent pant-type article 1 according to the invention, as is illustrated in FIG. 5. The method may include one or more of the steps of the method for forming a laminate web material for at least one of a front section or a back section a laminate web material for a disposable absorbent pant-type article disclosed herein, e.g. including one or more of steps S110-S160.

Steps M110, M120, M125 and M130 corresponds to steps S110, S120, S125 and S130. Please see the comments to steps S110-S130 disclosed herein.

In step M140, the continuous leg elastic member 511 is positioned in such a pattern that the leg cuts 527 made in step M190 will cut through the continuous leg elastic member 511. Hence, the continuous leg elastic member 511 is typically applied in an undulating way as seen in the cross direction. The wavelength A of the undulation corresponds to the width of the disposable absorbent pant-type article 1 as seen in the transverse direction T of the article 1. Further details of the undulation can be derived from the description herein of the first and second leg elastic members 25, 26 in the disposable absorbent pant-type article. Step M140 can be performed as described herein for step S140, but also other patterns for positioning the continuous leg elastic member 511 are feasible, as long as the leg cuts 527 made in step M190 will cut through the continuous leg elastic member 511.

Step M150 corresponds to S150. Please see the comments to steps S150 disclosed herein. In this step a first laminate web material 501 is formed, which is to form one of the front section 3 or the back section 4 of the disposable absorbent pant-type article 1, cf. step M160.

In step M170, a second laminate web material 521 is provided, which is to form the other of the front section 3 or the back section 4 of the disposable absorbent pant-type article 1. The second laminate web material may be formed in the corresponding steps of M110-M150. Alternatively, the second laminate web material may be made in any of a number of ways known from prior art, e.g. as the laminate web material 521 illustrated in FIG. 6, in which embodiment the second laminate web material 521 has no leg cut and is without leg elastic members.

In M180 absorbent assemblies 6 are provided. Each absorbent assembly 6 typically comprises a respective absorbent core 7. In that case, the absorbent assembly may comprise the absorbent core 7, a liquid permeable topsheet 73 and a liquid impermeable backsheet 72, wherein the absorbent core 7 typically is sandwiched between the liquid permeable topsheet 73 and the liquid impermeable backsheet 72. The absorbent core 7 may have any useful shape, such as hourglass shape, rectangular, T-shape, etc., as known in the art.

The front section 3 and the back section 4 may be formed by separate panels, i.e. a front body panel and a rear body panel, as illustrated in FIG. 6 and in the article of FIGS. 1-2a. In that case, step M180 comprises that the crotch region 5 is formed by a portion of the absorbent assembly 6, such that the portion of the absorbent assembly 6 spans the gap between the front body panel and the rear body panel.

Alternatively, the front section, the back section and the crotch section may be made of a chassis 2′ forming a single piece, as illustrated in FIG. 3. In that case, the first laminate web material and/or second laminate web material may be made or provided as part of the chassis 2′. It would also be possible to form the front section and the crotch section as a single piece and have a separate panel for the back section or to form the back section and the crotch section in a single piece and have a separate panel for the front section.

In step M190, leg cuts 527 are made such that the continuous leg elastic member 511 is cut. Thereby, in the article 1 being manufactured by the method, the first leg elastic member 25 ends at or in the vicinity of the first leg edge 18, and the second leg elastic member 26 ends at or in the vicinity of the second leg edge 19.

Step M200 is performed in order to prepare the precursor article 525 for applying the side seams 20, 21 in step M210. Finally, the disposable absorbent pant-type article 1 is separated from the following article in step M220.

Optionally, the method may further comprise cutting or deactivating the continuous leg elastic member 511 so as to form sets of elastic members 25, 26 separated by a non-elastic portion, where the non-elastic portion is intended to be located at the absorbent assembly 6, which is the case for the absorbent article 1 of FIGS. 1-2a.

Either the bottom layer web 507 or the top layer web 509 may face the wearer of the article.

The method may further comprise a step of providing a waist feature of the type described herein and/or as known from prior art. This step may e.g. be made just before step M150, in step M150 or just after step M150.

Optionally, step M140 comprises positioning the continuous leg elastic member, such that it extends for preferably at least 10 mm in a direction parallel with or substantially parallel with the machine direction in a first zone Z₁ along and adjacent to the first side edge 503 of the first laminate web material 501, for preferably at least 10 mm in a direction parallel with or substantially parallel with the machine direction MD in a second zone Z₂ spaced apart from the first zone Z₁, and both in the cross direction CD and the machine direction MD in a transfer zone Z_{T} located between the first and second zones Z₁, Z₂.

One flat apex 513 of the undulating wave is thus in the first zone Z₁, while the other flat apex 515 of the undulating wave is in the second zone Z₂. The wavelength of the undulating wave corresponds to the width W_{T} of the disposable absorbent pant-type article 1 as taken in transverse direction T. In between the first and second zones Z₁, Z₂, there is a transfer zone Z_{T}, in which the continuous leg elastic member 511 extends both in the cross direction CD and the machine direction MD. Further details of the undulation can be derived from the description herein of the first and second leg elastic members 25, 26 in the disposable absorbent pant-type article 1. When absorbent assemblies 6 are applied to the laminate web material 501 in step M180, the absorbent assemblies 6 are preferably positioned such that they overlap with the continuous leg elastic member 511 in the first zone Z₁. Furthermore, the leg cuts will preferably extend through the transfer zone Z_{T} and optionally into the second zone Z₂.

As illustrated in FIG. 4, step M140 optionally comprises positioning the continuous leg elastic member 511, such that it extends over less than 40% of the width W of the first laminate web material 501, preferably less than 35%, more preferably less than 30%.

As also illustrated in FIG. 4, the method comprises:
M125: Feeding a set of continuous elastic strands 517 in the machine direction MD, wherein step
M150 further comprises attaching the set 527 of continuous elastic strands between the bottom layer web 507 and the top layer web 509, thereby sandwiching the set of continuous elastic strands 517 between the bottom layer web 507 and the top layer web 509, such that the elastic strands of the set 517 are aligned substantially in parallel with each other and with the machine direction MD, and being spaced apart from each other in the cross direction CD. Please see the comments to step S125.

One or more of steps M110, M120, M125, M130 and M140 may be performed simultaneously or substantially simultaneously. They may also be performed in an arbitrary order.

In the illustrated embodiment, step M140 comprises positioning the continuous leg elastic member 511 in relation to the set of continuous elastic strands 517, such that it crosses at least one elastic strand of the set of continuous elastic strands 517. The crossed elastic strand may then form a leg side elastic strand 403, 404 of the type described herein, cf. FIG. 2a.

Optionally, step M140 comprises positioning the continuous leg elastic member 511 in relation to the set of continuous strands, such that in step M190 at least one elastic strand of the set of continuous elastic strands 517 ends at the leg cut 18, 19 without crossing the continuous leg elastic member 511, as in the article 1 illustrated in FIG. 2a.

Optionally, step M150 comprises bonding by applying adhesive to form adhesive bonds and/or by performing welding, e.g. ultrasonic welding and/or thermowelding.

Optionally, step M150 comprises applying adhesive to one or more of the bottom layer web 507, the top layer web 509, the continuous leg elastic member 511 and the set of continuous elastic strands 517.

Optionally, step M150 comprises applying adhesive to one or more of the bottom layer web 507 and the top layer web 509 in defined regions 531 overlapping the continuous leg elastic member 511.

### Combined Panel Variants

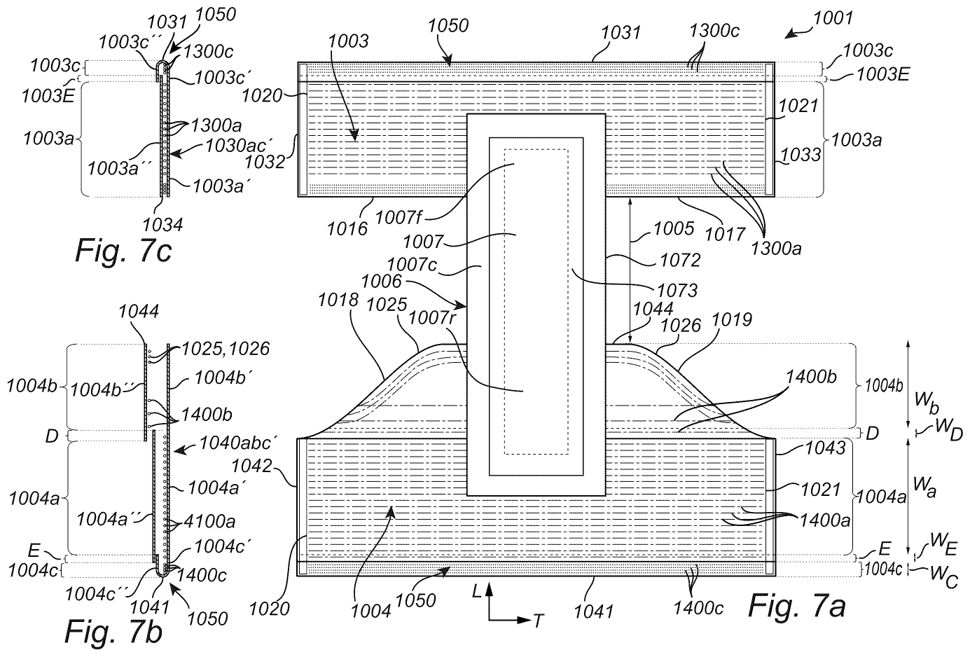

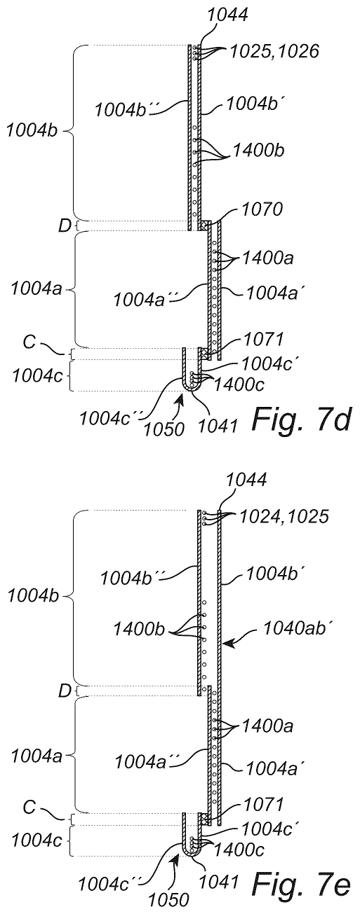

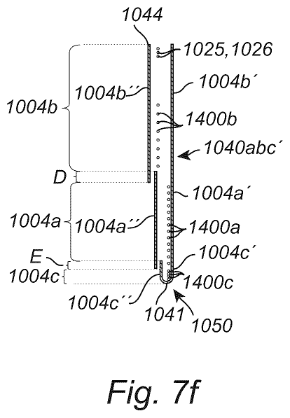

The embodiment illustrated in FIGS. 7a-7f have many features in common with the embodiment shown in FIGS. 1 and 2a. However, in this embodiment, at least one of the front section 1003 or the back section 1004 comprises or constitutes a combined panel. In the illustrated embodiment of FIG. 7a, it is the back section, referred to as the rear panel 1004, which comprises the combined panel.

The rear body panel 1004 of the variant in FIG. 7a is shown in section in FIG. 7b. Each leg elastic member, i.e. the first and the second elastic member 1025, 1026, does not extend all the way to the first rear panel side edge 1042 or the second rear panel side edge 1043, respectively. Hence, the first leg elastic member 1025 ends at or in the vicinity of the first leg edge 1018, and the second leg elastic member 1026 ends at or in the vicinity of the second leg edge 1019. Thus, each leg elastic member 1025, 1026 does not extend all the way to the first rear panel side edge 1042 or the second rear panel side edge 1043, respectively. Accordingly, each elastic member 1025, 1026 does not extend to the first side seam 1020 or the second side seam 1021, respectively. This provides for a possibility of making the second panel portion 1004b relatively shorter as seen in the longitudinal direction L, and thus to make the first panel portion 1004a relatively longer. A relatively longer first panel portion 1004a may be preferred since it is generally desired to increase the use of welding bonding techniques so as to diminish use of adhesive bonding techniques.

When comparing to the denotations used for the article of FIGS. 1 and 7a, front body panel 1003 corresponds to the front section 3 and the rear body panel 1004 corresponds to the back section 4.

Please see above for details of the absorbent core 1006, the topsheet 1073 and the backsheet 1072.

### Combined Panel

Thus, as explained herein, the example article 1001 depicted in FIGS. 7a-f is a version of a disposable pant-type article 1001 according to the invention, the article 1001 having a longitudinal direction L and a transverse direction T, and defining a thickness direction perpendicular to the longitudinal direction L and the transverse direction T, wherein an inside of the article as seen in the thickness direction is a wearer facing side of the article which will be facing towards the wearer when the article is worn. The outside of the article is a garment-facing side of the article which will be facing away from the wearer when the article is worn. The article 1001 comprises a front body panel 1003, a rear body panel 1004, and a crotch region 1005 being located between the front body panel 1003 and the rear body panel 1004. An absorbent assembly 1007 comprising an absorbent core 1006 is arranged with a central part 1007c of the absorbent assembly 1007 being located in the crotch region 1005. A first front panel side edge 1032 is joined to a first rear panel side edge 1042 in a first side seam 1020, and a second front panel side edge 1033 is joined to a second rear panel side edge 1043 in a second side seam 1021, and the article comprising a waist opening 1012, defined by a waist edge 1031 of the front panel 1003 and a waist edge 1041 of the rear panel 1004. Further, the front body panel 1003 forms a pair of leg edges 1016, 1017, and the rear body panel 1004 forms a pair of leg edges 1018, 1019, the pairs of leg edges of the front panel and the rear panel defining a pair of leg openings 1013, 1014.

Herein, each of the front body panel 1003 and the rear body panel 1004 has a top side towards one of the inside and outside of the article, and a bottom side towards the other one out of the inside and outside of the article 1001. “Top” and “bottom” is in this context not relating to any specific vertical or other orientation of the article 1001, but merely used to define two opposite directions along the thickness dimension of a body panel 1003, 1004.

At least one out of the front body panel 1003 and the rear body panel 1004 comprises a pair of leg elastic members 1025, 1026 arranged adjacent the corresponding pair of leg edges.

For example, and as exemplified in the variant of FIGS. 7a-f, the rear body panel 1004 of the absorbent article 1001 may comprise a pair of leg elastic members 1025, 1026 arranged adjacent the pair of leg edges 1018, 1019 of the rear body panel 1004 in the corresponding way as described for FIGS. 1-2a. In prior art solutions, it has been found to be challenging to apply leg elastic members 1025, 1026 which at least partially extends in a direction other than parallel with the transversal direction T using techniques involving welded bond patterns.

Thus, the present inventors have proposed, in some variants of the invention, a combined panel 1004 which comprises such leg elastic members 1025, 1026, and which comprises a first panel portion 1004a and a second panel portion 1004b, as seen along the longitudinal direction L. In the disclosed combined panel 1004, the first panel portion 1004a and the second panel portion 1004b are bonded using different bonding techniques, as will be further explained in the below.

Herein, the term “combined panel” is used to denote a panel comprising leg elastic members 1025, 1026 which at least partially extends in a direction other than parallel with the transversal direction T, and which comprises a first panel portion 1004a and a second panel portion 1004b being bonded using welding techniques and adhesive techniques, respectively, as defined in the appended claims. Variants and features of the combined panel will be further described in the below.

In the following, the description will be made in relation to a variant of an absorbent article 1001 as depicted in the FIGS. 7a-10, wherein the rear body panel 1004 is a combined panel comprising leg elastic members 1025, 1026, However, it should be understood that the below description of a combined panel may equally be applied to a front body panel 1003 being a combined panel comprising leg elastic members.

As exemplified in FIG. 7a and in FIG. 7b showing a cross section in a direction along the longitudinal axis L of the combined panel 1004 in FIG. 7a, the first panel portion 1004a comprises a first bottom layer 1004a′ and a first top layer 1004a″ being superimposed such that, in the first panel portion 1004a, the first bottom layer 1004a′ forms a bottom side surface and the first top layer 1004a″ forms a top side surface of the combined panel 1004, see FIG. 7b.

Similarly, the second panel portion 1004b comprises a second bottom layer 1004b′ and a second top layer 1004b″ being superimposed such that, in the second panel portion 1004b, the second bottom layer 1004b′ forms a bottom side surface and the second top layer 1004b″ forms a top side surface of the combined panel 1004.

The bottom side surface of the combined panel is the outermost surface of the panel as seen towards the bottom side of the panel, and the top side surface of the combined panel is the outermost surface of the panel as seen towards the top side of the panel. Accordingly, in each out of the first and second panel portion 1004a, 1004b, the respective bottom layer 1004a′, 1004b′ and top layer 1004a″, 1004b″ pair forms the bottom side surface and the top side surface of the combined panel 1004, i.e. forms the inside surface and the outside surface of the combined panel.

As will be further described in the below, the pairs of the first/second bottom layer 1004a′, 1004b′ and the first/second top layer 1004a″, 1004b″ are sandwiching elastomeric strands and/or elastic members to form the elastic rear panel 1004. By virtue of the top layer forming a top side surface and the bottom layer forming a bottom side surface of the combined panel 1004 in the first panel portion 1004a and in the second panel portion 1004b, the combined panel 1004 may be formed to be relatively thin and without using unnecessary amounts of material.

The first panel portion 1004a and the second panel portion 1004b are connected by a connection region D.

The connection region D joins the first panel portion 1004a and the second panel portion 1004b. Thus, in the connection region, the top and/or bottom layer of the first panel portion 1004a may for example be arranged so as to overlap the top and/or bottom layer of the second panel portion 1004b. Different variants of the connection region D will be more described in the below.

The first panel portion 1004a extends from the connection region D and in a longitudinal direction towards the waist edge 1041 of the combined panel 1004, and the second panel portion 1004b extends from the connection region D and in a longitudinal direction towards the leg edges 1018, 1019 of the combined panel 1004. For example, the second panel portion 1004b may extend longitudinally from the connection region D and all the way to the leg edges 1018, 1019 of the combined panel 1004.

The second panel portion 1004b comprises the leg elastic members 1025, 1026. In contrast, the first panel portion 1004a is free from leg elastic members 1025, 1026.

However, the first panel portion 1004a comprises a first set of elastomeric strands 1400a being sandwiched between the first bottom layer 1004a′ and the first top layer 1004a″. The elastomeric strands 1400a are aligned in parallel with each other and substantially with the transversal direction T, and the elastomeric strands 1400a are spaced apart from each other in the longitudinal direction. As such, the first set of elastomeric strands 1400a imparts elasticity in the transversal direction T to the first panel portion 1004a of the combined panel 1004.

In the first panel portion 1004a, the first bottom layer 1004a′ is bonded to the first top layer 1004a″ by a welded bond pattern for attaching the first set of elastomeric strands 1400a between the first bottom layer 1004a′ and the first top layer 1004a″. Since the first panel portion 1004a is free from leg elastic members 1025, 1026, i.e., includes only elastic strands 1400a which are substantially aligned with the transversal direction T, numerous techniques for bonding involving a welded bond pattern are available, which will be further described in the below. Thus, the first panel portion 1004a may bonded by a welded bond pattern and may be free from adhesive.

The second panel portion 1004b comprises the previously mentioned second bottom layer 1004b′, the second top layer 1004b″, and the leg elastic members 1025, 1026. In some variants, such as the one illustrated in FIGS. 7a and 7b, the second panel portion 1004b further comprises a second set of elastomeric strands 1400b. The leg elastic members 1025, 1026 and the second set of elastomeric strands 1400b are sandwiched between the second bottom layer 1004b′ and the second top layer 1004b″, the elastomeric strands 1400b being aligned in parallel with each other and substantially with the transversal direction T, and the elastomeric strands 1400b being spaced apart from each other in the longitudinal direction L.

Thus, the second set of elastomeric strands 1400b impart elasticity in a direction parallel to the transversal direction T to the second panel portion 1004b of the combined panel 1004.

The second panel portion 1004b comprises the leg elastic members 1025, 1026 arranged adjacent the leg edges 1018, 1019. The leg elastic members 1025, 1026 are sandwiched between the second top layer 1004b″ and the second bottom layer 1004b′, just as the optional elastomeric strands 1400b.

The second panel portion 1004b is bonded by adhesive bonds, wherein the adhesive bonds are applied to one or more out of the second top layer 1004b″, the second bottom layer 1004b′, the second set of elastomeric strands 1400b, and the leg elastic members 1025, 1026. To this end, prior known adhesive techniques may be used.

In view of the above, in the combined panel 1004, the first panel portion 1004a is bonded by means of a welded bond pattern, whereas the second panel portion 1004b is joined by means of adhesive. As such, adhesive bonding is used only where required, i.e., to accomplish the attachment of the leg elastics 1025, 1026. Since the leg elastics 1025, 1026 form a curved shape, other bonding methods than adhesive bonding methods may be less suitable in the portion 1004b of the combined panel 1004. By forming the combined panel 1004 of a first portion 1004a and a second portion 1004b being joined along a transversal connection region D, different bonding methods may be used in the first portion 1004a and in the second portion 1004b, without causing unnecessary consumption of material.

Optionally, and as in the illustrated variant, each leg elastic member 1025, 1026 crosses at least one elastic strand out of the second set of elastomeric strands 1400b.

In the first panel portion 1004a and in the second panel portion 1004b, respectively, the top layer 1004a″, 1004b″ forms a top side surface and the bottom layer 1004a′, 1004b′ forms a bottom side surface of the combined panel 1004. That no additional layers of web material are present in the combined panel 1004 over the first panel portion 1004a and the second panel portion 1004b may be advantageous in terms of breathability, comfort, sustainability, and economics.

The connection region D serves the purpose of connecting the first and second panel portions only and may thus be made so as to have a length WD being as short as possible. In the connection region D, the combined panel 1004 may comprise more than two layers, for example three or more layers, to provide the desired connection between the first and second panel portions.

For example, the connection region D may have a longitudinal length W_{D} being no more than 10% of a total longitudinal length of the combined panel, such as no more than 7% or no more than 5% of a total longitudinal length W of the combined panel. In the illustrated example, the connection region D may have a longitudinal length W_{D} corresponding to from 4 to 5% of the longitudinal length W of the combined panel 1004.

The first bottom layer 1004a′, the first top layer 1004a″, the second bottom layer 1004b′ and the second top layer 1004b″ may be selected so as to provide the combined panel 1004 with a relatively uniform appearance, despite the division into a first and a second panel portion 1004a, 1004b, and the presence of the connection region D.

In some variants, such as in the examples of FIG. 7b, the first bottom layer 1004a′ and the second bottom layer 1004b′ are both formed by the same, single sheet of material 1040abc′, extending longitudinally over the connection region D.

The first top layer 1004a″ and the second top layer 1004b″ may advantageously be formed by separate sheets of sheet material, as in the variant of FIG. 7b. As such, in production, each of the separate sheets may be bonded to form the first portion 1004a or the second portion 1004b, respectively, with the respective welding or adhesive bonding process.

Still, the second top layer 1004b″ and the first top layer 1004a″ may be formed by sheets of identical sheet material. For example, the second top layer 1004b″ and the first top layer 1004a″ may be formed by separate sheets being slitted from the same original (broader) sheet material.

Variants where the first bottom layer 1004a′ and the second bottom layer 1004b′ are formed by a single sheet of material 1040ab′, 1040abc′ extending over the connection region D have the further advantage that the connection region D may comprise relatively few layers of sheet material. For example, when as in FIG. 7b the sheet material of the first top layer 1004a″ and the sheet material of the second top layer 1004b″ are arranged so as to overlap in the connection region D, the connection region still comprises no more than three layers of sheet material.

Thus, the combined panel 1004 may comprise a first panel portion 1004a and a second panel portion 1004b connected by the connection region D as set out in the above, wherein the first panel portion 1004a and the second panel portion 1004b each comprises no more than two layers of sheet material, and the connection region D comprises no more than three layers of sheet material. Accordingly, the connection region D may be made with a lesser impact on the breathability and comfort of the combined panel 1004.

The first panel portion 1004a has a longitudinal length Wa which may be a significant portion of the total length W of the combined panel. For example, the longitudinal length Wa may be at least 20% of a total longitudinal length of the combined panel, such as at least 25%, or at least 30%. In the illustrated example, the longitudinal length Wa of the first panel portion 1004a is from 30% to 40% of the longitudinal length W of the combined panel 1004. However, it will be understood that this proportion may be increased or decreased for example depending on the size and model of the absorbent article.

The second panel portion 1004b has a second longitudinal length Wb which may be a significant portion of the total length W of the combined panel 1004. For example, the longitudinal length Wb of the second panel portion 1004b may be at least 20%, such as at least 25% or at least 30% off a total longitudinal length of the combined panel 1004. For example, the second panel portion may have a longitudinal length being less than 70% of the total longitudinal length of the combined panel 1004. For example, the second panel portion may have a longitudinal length being from 25% to 70% of the total longitudinal length of the combined panel 1004.

In the absorbent article 1001, the absorbent assembly 1007 may be arranged with a central part 1007c of the absorbent assembly 1007 being located in the crotch region 1005 and with a front end part 1007f of the absorbent assembly 1007 overlapping with the front body panel 1003 in a front overlap area and with a rear end part 1007r of the absorbent assembly 1007 overlapping with the rear body panel 1004 in a rear overlap area.

As exemplified in FIG. 7a, the absorbent assembly 1007 may be arranged so as to overlap a part of the connection region D. In other words, the rear end part 1007r of the absorbent assembly 1007 extends longitudinally beyond the transversal connection region D in a direction towards the rear waist edge 1041 of the article 1001.

Further, the pant-type article 1001 may have an elastic waist feature 1050 arranged along the waist opening 12. An elastic waist feature 1050 may include one or more elastic elements extending parallel with the front panel waist edge 1031 and the rear panel waist edge 1041. The elastic waist element or elements may be incorporated in the combined panel 1004 or may be applied as a separate waistband which is attached to the front panel waist edge 1031 and the rear panel waist edge 1041 or which is attached to the front and/or to the rear body panel 1003, 1004 with the waistband completely overlapping with the corresponding body panel 1003, 1004 and sharing a common waist edge. The pant-type article 1001 which is shown in FIGS. 7a-10 has an elastic waist feature 1050 which extends around the full circumference of the waist opening 12. However, an elastic waist feature may be arranged only along a part of the waist opening, such as only along the rear panel waist edge, only along the front panel waist edge or along a part of one or both of the front panel waist edge and the rear panel waist edge, which part has a length which is less than the full length of the corresponding panel waist edge.

For example, to form an elastic waist feature 1050, the combined panel 1004 may comprise a waist panel portion 1004c as exemplified in FIG. 7b.

In the waist panel portion 1004c, a waist bottom layer 1004c′ and a waist top layer 1004c″ are superimposed such that the waist bottom layer 1004c′ forms a bottom side surface and the waist top layer 1004c″ forms a top side surface of the combined panel 1004.

The waist panel portion 1004c further comprises a third set of elastomeric strands 1400c being sandwiched between the waist bottom layer 1004c′ and the waist top layer 1004c″, the elastomeric strands 1400c being aligned substantially in parallel with each other and with the transversal direction T, and the elastomeric strands 1400c being spaced apart from each other in the longitudinal direction L.

The waist panel portion 1004c is joined to the first panel portion 1004a by a waist connection region E. As such, the first panel portion 1004a extends longitudinally from the connection region D to the waist connection region E. The waist panel portion 1004c extends from the waist connection region E to the waist edge 1041 of the combined panel 1004.

Similar to the connection region D, it is preferred that the waist connection region E has a relatively short longitudinal length W_{E}, for example no more than 10% or no more than 5% of the total longitudinal length W of the combined panel 1004.

Numerous options for forming a waist panel portion 1004c on a front or rear body panel are known and may be combined with the present disclosure. For example, the different variants described in WO 2018/0971071 A1 may be used on the front or rear body panels as described herein. For example, the different variants as described in EP 3 788 997 A1 may be used on the front or rear body panels as described herein.

For example, as depicted in the variant of FIG. 7b, the waist bottom layer 1004c′ and the waist top layer 1004c″ may be formed from a continuous material sheet being folded upon itself along the waist edge 1041.

Since the waist panel portion 1004c is free from elastic members extending in directions other than substantially parallel to the transversal direction T, the waist panel portion 1004c may advantageously be bonded by welded bond pattern bonding the waist bottom layer 1004c′ to the waist top layer 1004c″ so as to attach the third set of elastic strands 1400c therebetween. Again, this implies that adhesives are used in the combined panel 1004 only when required by the need for securely bonding leg elastic members, and that adhesives are otherwise avoided.

In some variants, such as exemplified in FIG. 7f, the waist bottom layer 1004c′, the waist top layer 1004c″, the first bottom layer 1004a′ and the second bottom layer 1004b′ may be formed by a single continuous material sheet 1040abc′. As such, a uniform appearance of the bottom side of the panel may be achieved, being particularly desired when the bottom direction is the outward direction of the article 1001. Moreover, advantages may be obtained in manufacturing as will be described in the below. To form the waist top layer 1004c″ from the continuous material sheet 1040abc′, the sheet 1040abc′ is folded along a folded edge thus forming the waist edge 1041 of the combined panel 1004.

In the variant of FIG. 7b, the first top layer 1004a″ and the second top layer 1004b″ are formed by separate material sheets which overlap in the connection region D. Similarly, the material sheets of the first top layer 1004a″ and the waist top layer 1004c″ overlap in the longitudinal direction L in the waist connection region E.

FIGS. 7d, 7e, and 7f illustrate different examples of possible cross-sections through a variants of a combined panel 1004 as generally illustrated of FIG. 7a, as seen along the longitudinal direction L, thus illustrating different variants of connection regions D and waist features 1050.

In the variant of FIG. 7d, the first bottom layer 1004a′ and the second bottom layer 1004b′ are formed by separate material sheets. Moreover, the first top layer 1004a″ and the second top layer 1004b″ are formed by separate material sheets. The connection region D comprises a joint 1070 connecting the first panel portion 1004a and the second panel portion 1004b. As may be seen in FIG. 7d, the joint 1070 may for example be applied such that the first top layer 1004a″ and the first bottom layer 1004a′ overlap with the second top layer 1004b″ and the second bottom layer 1004b′ in the connection region D. The joint 1070 may be any suitable joint, such as an adhesive joint or a welded joint.

Further, the combined panel 1004 in the variant of FIG. 7d comprises a waist feature 1050 in the form of a waist panel portion 1004c. In the waist panel portion 1004c, a waist bottom layer 1004c′ and a waist top layer 1004c″ are superimposed such that the waist bottom layer 1004c′ forms a bottom side surface and the waist top layer 1004c″ forms a top side surface of the combined panel 1004.

The waist panel portion 1004c further comprises a third set of elastomeric strands 1400c being sandwiched between the waist bottom layer 1004c′ and the waist top layer 1004c″, the elastomeric strands 1400c being aligned substantially in parallel with each other and with the transversal direction T, and the elastomeric strands 1400c being spaced apart from each other in the longitudinal direction L.

For example, as depicted in the variant of FIG. 7d, the waist bottom layer 1004c′ and the waist top layer 1004c″ may be formed from a continuous material sheet being folded upon itself along the waist edge 1041.

The waist panel portion 1004c is joined to the first panel portion 1004a by a waist connection region E.

Since the waist panel portion 1004c is free from elastic members extending in directions other than substantially parallel to the transversal direction T, the waist panel portion 1004c may advantageously be bonded by welded bond pattern bonding the waist bottom layer 1004c′ to the waist top layer 1004c″ so as to attach the third set of elastic strands 1400c therebetween. Again, this implies that adhesives are used in the combined panel 1004 only when required by the need for securely bonding leg elastic members, and that adhesives are otherwise avoided.

The waist connection region E may be made in any suitable manner. For example, as in the variant illustrated in FIG. 7d, the waist connection regionE may comprise a joint 1071, which may be a joint such as described in relation to the joint 1070 of the connection region D in the above.

For example, and as illustrated in FIG. 7d, the waist bottom layer 1004c′ and the waist top layer 1004c″ may be arranged so as to overlap with the first bottom layer 1004a′ and the first top layer 1004a″ in the waist connection region E.

As mentioned in the above, similar to the connection region D, it is preferred that the waist connection region E has a relatively short longitudinal length WE, for example no more than 10% or no more than 5% of the total longitudinal length W of the combined panel 1004.

In the example of FIG. 7d, each one out of the second panel portion 1004b, the first panel portion 1004a, and the optional waist panel portion 1004c may be produced separately and then joined via the joints 70, 1071 to form the combined panel 1004. As such, it may be advantageous to form a second laminate web 1004b, a first laminate web 1004a, and optionally a waist laminate web, and to join the webs via joints 70, 31 so as to form a combined laminate web having a cross-section as illustrated in FIG. 7d, and which may be cut into rear body panels 1004 during manufacture of the pant-type articles 1001.

FIG. 7e illustrates another variant of a combined panel 1004 as seen in a cross-section along the longitudinal direction of FIG. 7a.

In this variant, the second bottom layer 1004b′ of the second panel portion 1004b and the first bottom layer 1004a′ of the first panel portion 1004a are formed by a single continuous material sheet 1040ab′, extending over the connection region D. As seen in FIG. 7e, in such variants, a combined bottom layer 1040ab′ constitutes the second bottom layer 1004b′ in the second portion 1004b of the combined panel 1004 and the first bottom layer 1004a′ in the first portion 1004a of the combined panel 1004. This gives the combined panel 1004 a uniform appearance when seen from the bottom layer side. For example, as in the illustrated variant, the bottom direction may be the outer direction of the article 1001, in which case the uniform appearance of the combined bottom layer 1040ab′ will be visible from the outside of the article 1001. Thus, any joints or overlap layers may be avoided on the outside of the article 1001. Further, as will be explained in the below, the second bottom layer 1004b′ and the first bottom layer 1004a′ being formed by a single continuous material sheet may provide for advantageous manufacturing.

As illustrated in FIG. 7e, the first top layer 1004a″ of the first panel portion 1004a, and the second top layer 1004b″ of the second panel portion 1004b are formed by separate material sheets which are arranged with an overlap the longitudinal direction L in the connection region D.

In the example of FIG. 7e, the combined panel 1004 further comprises a waist feature 1050. The waist feature 1050 may be any of the waist features as described in the above in relation to the example of FIG. 7b or FIG. 7d. For example, as in the variant of FIG. 7e, the waist feature 1050 may comprises a waist panel portion 1004c being joined to the first panel portion 1004a via a joint 1071, similar to what has already been described in relation to FIG. 7d.

Other variants than those depicted in FIGS. 7a-10 are or course possible. For example, the waist bottom layer 1004c′ and the first bottom layer 1004a′ may be formed by a single continuous material sheet as described in relation to FIG. 7b, even in variants where the second panel portion 1004b and the first panel portion 1004a do not share a continuous material sheet, but rather are joined by a joint 1070 as described in relation to FIG. 7d.

When the connection region D or the waist connection region E involves an overlap such as an overlap between separate sheet materials of the top layers only, the overlap could be made in any direction.

For example, FIG. 7f illustrates a variant largely similar to the variant of FIG. 7b, apart from that in this variant, in the waist connection region E, the first top layer 1004a″ is arranged to the top side of the waist top layer 1004c″, whereas in FIG. 7b, the first top layer 1004a″ is arranged to the bottom side of the waist top layer 1004c″. The relative arrangement of the layers in the waist connection region E may reflect the order in which the waist portion 1004c and the first panel portion 1004b, respectively, was formed when manufacturing the combined panel 1004.

Different relative arrangements of the first top layer 1004a″ and the second top layer 1004b″ are also possible in the connection region D. However, in the connection region D it may be preferable if the second top layer 1004b″ is arranged to the top side of the first top layer 1004a″ since this implies that the welded pattern of the first panel portion 1004a may be produced prior to the adhesive of the second panel portion 1004b, using a continuous material 1004ab′ to form the first bottom layer 1004a′ and the second bottom layer 1004b′. Accordingly, any risks of having adhesive in the welding equipment may be avoided.

Moreover, the connection region D and/or the waist connection region E could be close to infinitely small. For example, in a variant where a first top layer 1004a″ is arranged edge to edge with a second top layer 1004b″ on a continuous material 1004ab′ forming the first bottom layer 1004a′ and the second bottom layer 1004b′, the connection region D would comprise only the line between the edges of the first bottom layer 1004a′ and the second bottom layer 1004b′.

The first bottom layer 1004a′, the first top layer 1004a″, the second bottom layer 1004b′ and the second top layer 1004b″ may be selected so as to provide the combined panel 1004 with a relatively uniform appearance, despite the division into a first and a second panel portion 1004a, 1004b, and the presence of the connection region D.

In some variants, such as in the examples of FIG. 7b, FIG. 7e, and FIG. 7f, the first bottom layer 1004a′ and the second bottom layer 1004b′ may be formed by a single sheet of material 1040ab′, 1040abc′ extending over the connection region D.

In other variants, such as in the example of FIG. 7d, the second bottom layer 1004b′ and the first bottom layer 1004a′ may be formed by different sheets of material 1004a′, 1004b′. Although giving a less uniform appearance than the versions of FIG. 7b, FIG. 7e and FIG. 7f, still, in this case, the different sheets may be made of identical sheet material. For example, the first bottom layer 1004a′ and the second bottom layer 1004b′ may be formed by separate sheets being slitted from the same original (broader) sheet material.

The first top layer 1004a″ and the second top layer 1004b″ may advantageously be formed by separate sheets of sheet material, as in the variants of the Figures. As such, in production, each of the separate sheets may be bonded to form the first portion 1004a or the second portion 1004b, respectively, with the respective welding or adhesive bonding process.

Still, the second top layer 1004b″ and the first top layer 1004a″ may be formed by sheets of identical sheet material. For example, the second top layer 1004b″ and the first top layer 1004a″ may be formed by separate sheets being slitted from the same original (broader) sheet material.

Variants where, as in FIG. 7b, FIG. 7e or FIG. 7f, the first bottom layer 1004a′ and the second bottom layer 1004b′ are formed by a single sheet of material 1040ab′, 1040abc′ extending over the connection region D have the further advantage that the connection region D may comprise three layers of sheet material only.

Although in the illustrated variant, the rear panel 1004 only comprises leg elastic members and is a combined panel, thus comprising a second panel portion 1004b and a rear panel portion 1004a as described in the above, it will be understood that other variants are possible. For example, also the front panel 1003 may comprise leg elastic members and/or may be a combined panel comprising a first panel portion and a second panel portion as described in the above.

In some variants however, such as in the variant of FIG. 7a, the front body panel 1003 of the absorbent article is free from leg elastic members 1025, 1026. As seen in FIG. 7a and FIG. 7c, FIG. 7c showing a cross-section in the longitudinal direction L of the front body panel 1003 in FIG. 7a, the front body panel 1003 comprises a main bottom layer 1003a′ and a main top layer 1003a″ and a set of elastomeric strands 300b being sandwiched between the main bottom layer 1003a′ and the main top layer 1003a″, the elastomeric strands 1300a being aligned in parallel with each other and substantially with the transversal direction T, and the elastomeric strands 1300a being spaced apart from each other in the longitudinal direction L.

In the main portion 1003a, the main bottom layer 1003a′ is bonded to the main top layer 1003a″ by a welded bond pattern for attaching the set of elastomeric strands 1300a.

In the main portion 1003a, the main bottom layer 1003a′ forms a bottom side surface and the main top layer 1003a″ forms a top side surface of the front body panel 1003.

The front body panel 1003 may be provided with a waist feature 1050, similar to what has been described in the above. In the variant of FIG. 7c, the front body panel 1003 comprises a waist panel portion 1003c comprising a top waist panel layer 1003c″ and a bottom waist panel layer 1003c′, where the bottom waist panel layer 1003c′ is formed by a combined bottom panel sheet 30ac′, which also forms the main bottom layer 1003a′, similar to the waist feature 1050 as described in relation to FIGS. 7b and 7c.

Preferably, the waist panel portion 1003c of the front body panel 1003 may be bonded by a welded bond pattern as described in the above in relation to the combined panel 1004.

The front body panel 1003 being free from leg elastic members 1025, 1026 extending in a direction other than parallel with the transversal direction T implies that the front body panel 1003 as a whole may suitably be bonded by a welded bond pattern. Thus, the front body panel 1003 may be free from adhesive.

In some variants, as depicted in FIGS. 7a and 7c, the set of elastomeric strands 1300a in the main portion 1004a may be divided into a first subset and a second subset, where the first subset of elastomeric strands differs from the second subset of elastomeric strands as regards longitudinal spacing between the elastic strands, and/or features of the elastic strands such as diameter of the elastic strands. For example, the first subset of elastomeric strands may be applied over a majority of the longitudinal extension of the main panel portion 1003a, whereas the second subset of elastomeric strands is applied adjacent the leg edges 1016, 1017 of the front panel portion 1003. As such, the second subset of elastomeric strands may be provided to ensure a good fit of the front panel 1003 at the leg openings 1013, 1014 of the absorbent article 1001. For example, the second subset of elastomeric strands may have a longitudinal spacing between each strand being less than the longitudinal spacing of the first subset of elastomeric strands. The second subset of elastomeric strands may comprise a relatively low number of elastomeric strands, such as from 1 to 5 strands, or, as in the example, from 2 to 4 strands.

In other variants, the front panel 1003 may be provided with a leg panel portion which may be formed in the same manner as previously described for the waist panel portion, to ensure a good fit of the front panel 1003 at the leg openings 1013, 1014 of the absorbent article. Thus, a leg panel portion may be connected to the main panel portion 1003a via a leg connection portion, similar to the waist connection portion C described in the above.

In the variant of FIGS. 7a and 7b, the second panel portion 1004b comprises a second set of elastic strands 1400b including a relatively low number of strands 1400b. However, the second set of elastic strands 1400b may optionally be completely omitted such that the second panel portion 1004b comprises no elastic elements but the leg elastic members 1025, 1026.

### Combined Laminate Material Web

In in-line manufacturing of absorbent articles 1001 being of the kind comprising the combined panel, the front body panel 1003 and the rear body panel 1004 of the absorbent article 1001 as depicted in FIG. 7a may be achieved by cutting the front body panel and the rear body panel from a respective front body panel web or a rear body panel web.

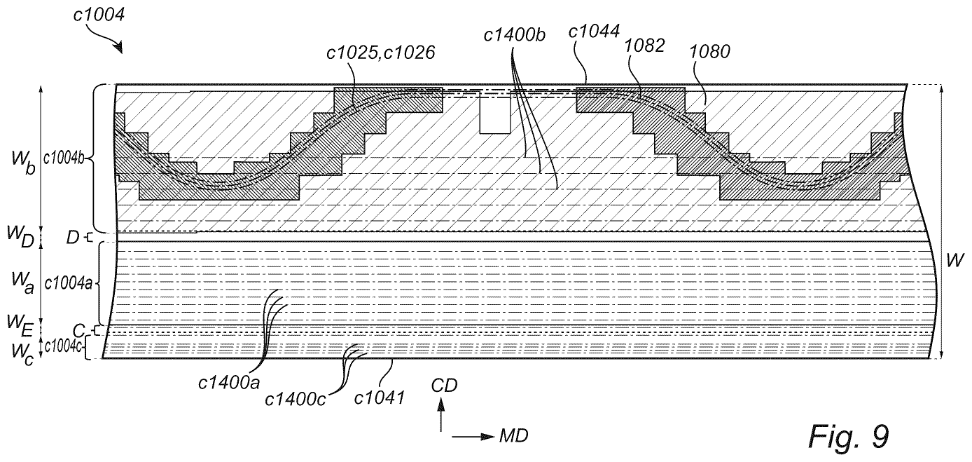

FIG. 9 schematically illustrates a variant a combined laminate material web c1004 which may be cut for forming combined body panels 4 as described in the above. The illustrated combined laminate material web c1004 is for forming combined panels 4 in accordance with the variant of FIGS. 7a and 7b. However, it will be understood that other variants of combined laminate material webs may be formed, such as variants corresponding to the other variants of combined panels illustrated herein.

Thus, FIG. 9 illustrates a variant of a combined laminate material web c1004 for a disposable hygienic article, the combined laminate material web being continuous as seen in a machine direction MD and having a width W between two side edges 1500, 1501 in a cross-direction CD, perpendicular to the machine direction MD, the combined laminate material web defining a top direction being perpendicular to the machine direction and to the cross-direction CD, and a bottom direction opposing the top direction. The combined laminate material web c1004 comprises a first web portion c1004a and a second web portion c1004b, the first web portion c1004a and the second web portion c1004b being connected by a connection region D extending along the machine direction MD, such that the first web portion c1004a extends from the connection region D and in the cross-direction towards the first side edge c1041 of the combined laminate material web, and the second web portion c1004b extends from the connection region D and in the cross-direction towards the second side edge c1044 of the combined laminate material web.

The first web portion c1004a comprises a first bottom layer 1004a′ and a first top layer 1004a″ being superimposed such that, in the first web portion 1004a, the first bottom layer 1004a′ forms a bottom side surface and the first top layer 1004a″ forms a top side surface of the combined laminate material web c1004.

Similarly, the second web portion 1004b comprises a second bottom layer 1004b′ and a second top layer 1004b″ being superimposed such that, in the second web portion 1004b, the second bottom layer 1004b′ forms a bottom side surface and the second top layer 1004b″ forms a top side surface of the combined laminate material web c1004.

The first web portion c1004a further comprises a first set of elastomeric strands c1400a being sandwiched between the first bottom layer 1004a′ and the first top layer 1004a″, the elastomeric strands c1400a being aligned in parallel with each other and substantially with the machine direction MD, and the elastomeric strands being spaced apart from each other in the cross-direction CD, and the first web portion c1004a is bonded by the first bottom layer 1004a′ being bonded to the first top layer 1004a″ by a welded bond pattern for attaching the first set of elastomeric strands 1400a between the first bottom layer 1004a′ and the first top layer 1004a″.

The second web portion c1004b comprises a second set of elastomeric strands 1400b being sandwiched between the second bottom layer 1004b′ and the second top layer 1004a′, the elastomeric strands 1400b being substantially aligned in parallel with each other and with the machine direction MD, and the elastomeric strands 1400b being spaced apart from each other in the cross-direction CD. The second web portion 1004b further comprising additional elastic members c2500, c2600 extending at least partially in a direction other than parallel with machine direction MD, wherein the additional elastic members 1025, 1026 are sandwiched between the second bottom layer 1004b′ and the second top layer 1004b″, and the second web portion 1004b is bonded by the second bottom layer 1004b′ being bonded to the second top layer 1004b″ by adhesive bonds, for attaching the second set of elastomeric strands 1400b and the additional elastic members 1025, 1026 between the second bottom layer 1004b′ and the second top layer 1004b″.

The additional elastic members c2500, c2600 of the second web portion c1004b are to form the leg elastic members 1025, 1026 in the absorbent articles 1001 including combined panels 1004 cut from the combined laminate web c1004. To that end, the additional elastic members c2500, c2600 may, as in the illustrated variant, run continuously and in a meandering pattern over the second web portion 1004b of the combined laminate web c1004.

As will be described in more detail in the below in relation to a method for forming an absorbent article 1001, the manufacturing of an absorbent article 1001 will involve cutting portions of the combined laminate web c1004 away so as to form the leg edges 1018, 1019 of the combined panels 4, and cutting the combined laminate web c1004 into separate rear panels 4 by cuts along the cross-direction along the dotted lines in FIG. 9. Thus, in the combined panels 4, the additional elastic members c2500, c2600 will be positioned adjacent the leg edges 1018, 1019. Finally, the additional elastic members c2500, c2600 which may run continuously as seen in the machine direction MD of the combined laminate web c1004 may be centrally cut or otherwise de-elastified in a central section of each rear panel 1004, so as to form a first elastic members 25 along a first leg edge 1018, and a second elastic member 1026 along a second leg edge 1019 in the final rear panel 1004. Such cutting or de-elastification of the additional elastic members c2500, c2600 in a central section of each rear panel 1004 may be performed prior to cutting the combined laminate web c1004 into separate rear panels 4.

As explained in the above in relation to the article 1001 comprising the rear panel 1004, a combined laminate web 4 with a first web portion 1004a and a second web portion 1004b as described in the above may be constructed in different manners, such as exemplified in relation to FIGS. 7d to 7b, or 7f, or in relation to FIG. 6. Thus, although FIG. 9 corresponds to a combined laminate web having a cross-section such as illustrated in FIG. 7d, it will be understood that the combined laminate web c1004 may be formed in accordance with any one of the variants of FIGS. 7d to 7b, or 7f.

As is clear to a person skilled in the art, the features and advantages as described in the above in relation to the exemplary combined panels 4 are equally applicable to a combined laminate web c1004.

### Method for Forming a Combined Laminate Web

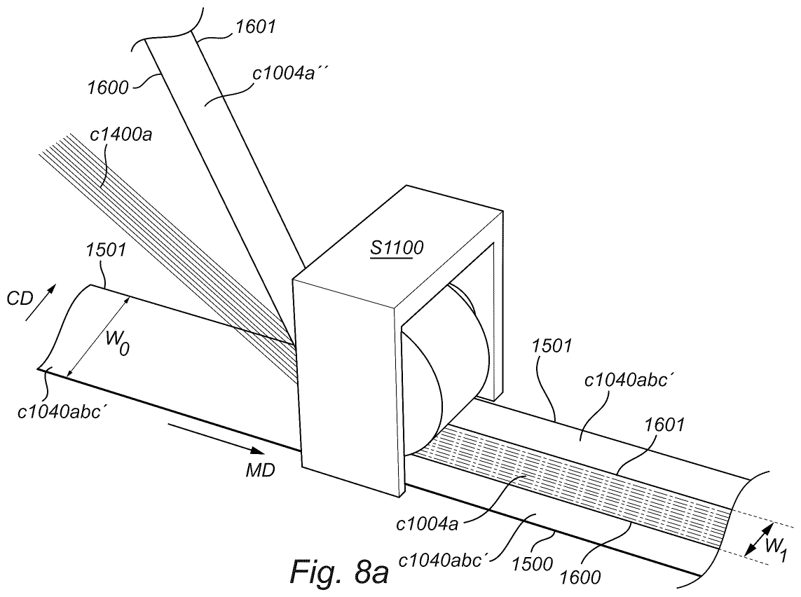

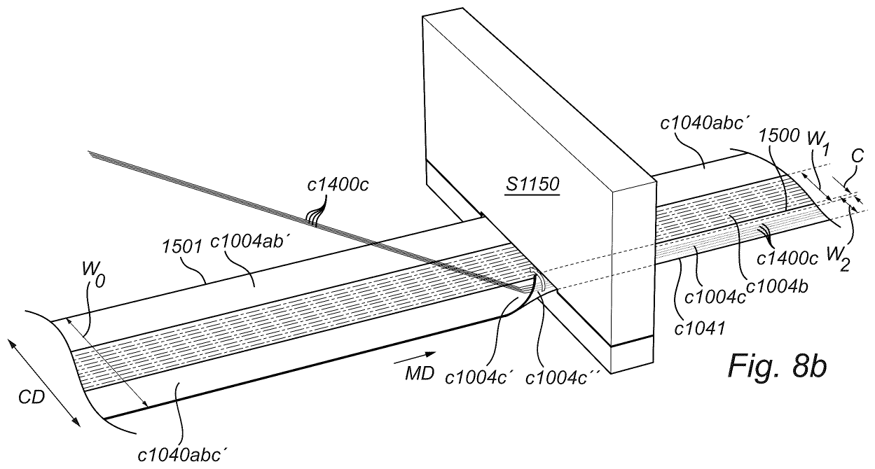

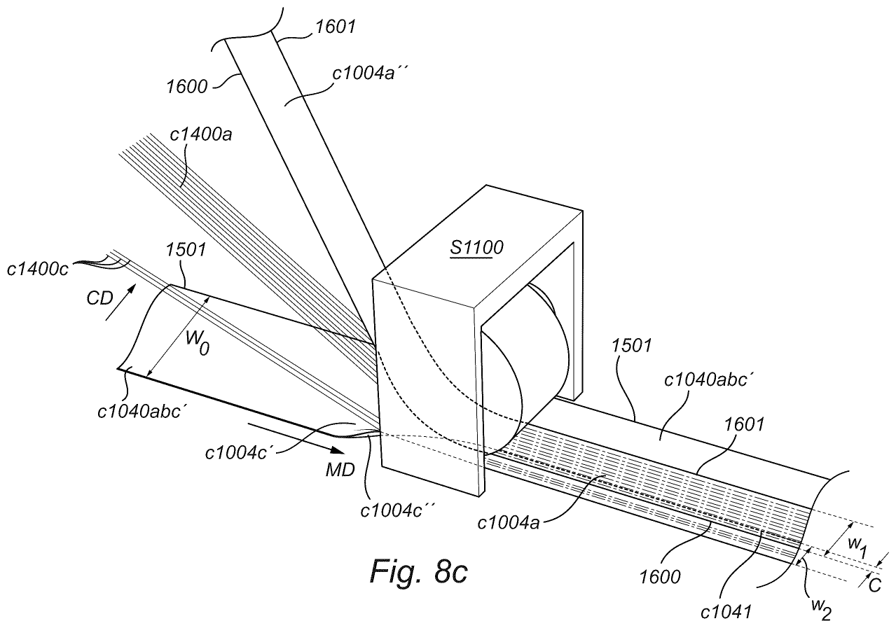

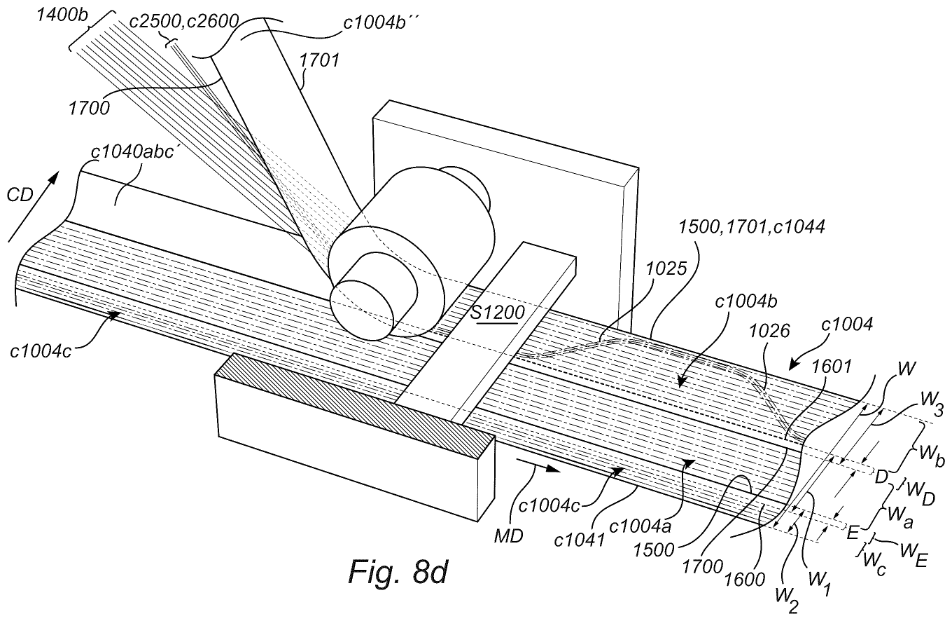

FIGS. 8a, 8b, and 8d together illustrate a first variant of a continuous method for forming a combined laminate web for a disposable hygienic article.

The illustrated variant of a method is for forming a combined laminate material web c1004 as illustrated in FIG. 9, and with a cross-section as illustrated in FIG. 7b. This variant is believed to be advantageous from manufacturing point of view and for providing rear panels 4 with a uniform outer appearance. However, features described in relation to this specific variant may be equally applicable to other variants of the method for forming a combined laminate material web, for example in accordance with any one of the cross-sections illustrated in FIG. 7d, 7e or 7f.

Thus, the method to be described is a method for forming a combined laminate material web c1004 for a disposable hygienic article, the combined laminate material web being continuous as seen in a machine direction MD and having a width W between two side edges c1041, c1044 in a cross-direction CD perpendicular to the machine direction MD. The combined laminate material having a top side and a bottom side as seen in a thickness direction being perpendicular to the machine direction MD and to the cross-direction CD.

FIG. 8a schematically illustrates a welding bonding step S1100 of the method. FIG. 8b shows a continuous combined bottom layer web c1040abc′ which is being advanced in a machine direction MD. In the illustrated variant, the combined bottom layer web c1040abc′ is to form both the first bottom layer 1004a′ of the first web portion c1004a and the second bottom layer 1004b′ of the second web portion c1004b of the combined web laminate c1004 to be formed. Further, a continuous first top layer web c1004a″ is being advanced in the machine direction MD. FIG. 8a also shows a first set of continuous elastic strands c1400a being advanced in a machine direction MD so as to be sandwiched between the continuous combined bottom layer web c1040abc′ and the continuous first top layer web c1004a″.

The combined bottom layer web c1040ab′ has a combined width WO in a direction cross the machine direction between first longitudinal edge 1500 and a second longitudinal edge 1501. The first top layer web c1004a″ has a first width W1 in a direction cross the machine direction between a second and a first longitudinal edge 1600, 1601. As illustrated in the Figs., the first width W1 is less than the combined width W0.

In a welding bonding step S1100, which is only schematically illustrated, the combined bottom layer web c1040abc′ is bonded to the first top layer web c1004a′ by a welded bond pattern, with the first set of elastomeric strands c1400a being sandwiched between the combined bottom layer web c1004b′ and the first top layer web c1004a′, so as to be aligned in parallel with each other and substantially with the machine direction.

As may be seen in FIG. 8a, in this variant the first top layer web c1004a″ is bonded to the combined bottom layer web c1040abc′ such that, in the cross-direction CD, a first portion of the combined bottom layer web c1040abc′ extends beyond the first top layer web c1004a″ towards the first side edge 1500 of the combined bottom layer web c1040abc′. As will be explained in the following, this first portion of the combined bottom layer web c1040abc′ will be used to form a waist feature 1050 of the combined laminate web c1004. Thus, if selecting to use another form of waist feature 1050, in other variants the first top layer web c1004a″ could for example be arranged with a first longitudinal edge 1600 coinciding with the first side edge 1500 of the combined bottom layer web c1040abc′.

Also, in this variant, the first top layer web c1004a″ is bonded to the combined bottom layer web c1040abc′ such that, in the cross-direction CD, a second portion of the combined bottom layer web c1040abc′ extends beyond the first top layer web c1004a″ towards the second side edge 1500 of the combined bottom layer web c1040abc′. As will be described in the following, this second portion of the combined bottom layer web c1040abc′ will be used to form the second bottom layer 1004b′ of the second web portion c1004b of the combined laminate web c1004.

FIG. 8b shows a variant of a waist forming step S1150 for forming a waist laminate web portion c1004c of the laminate web c1004.

In this variant, the first portion as mentioned in the above of the combined bottom layer web c1040abc′ is folded along the machine direction MD, such that the first portion forms, on each side of the fold, a waist bottom layer c1004c′ and a waist top layer c1004c″ in a waist web portion c1004c of the combined laminate web 4.

As illustrated in FIG. 8b, a waist portion set of continuous elastic strands c1400c is being advanced in a machine direction MD so as to be sandwiched between the waist bottom layer c1004c′ and the waist top layer c1004c″.

As illustrated in FIG. 8b, the folded portion forming the waist bottom layer c1004c′ has a second width W2 in the cross-direction, extending from the fold to the first side edge 1501 of the combined bottom layer web c1040abc′. The second width W2 may be such that, in the connection region C, the waist top layer c1004c″ overlaps with the first top layer c1004a″.

The waist bottom layer c1004c′ and the waist top layer c1004c″ are bonded by a welding step S1150 providing a welded bond pattern to attach the third set of elastic strands c1400c between the waist bottom layer c1004c′ and the waist top layer c1004c″. As such, the waist forming step S1150 may advantageously be performed upstream of the adhesive bonding step S1200, which will be described in further detail in the below.

In the illustrated variant, the waist forming step S1150 takes place downstream of the welding step S1100 for forming the first web portion 1004a. However, in other variants, the waist forming step S1150 may take place upstream of the welding step S1000. In such a variant, in the connection region C, the first top layer web c1004a″ would be arranged to the top side of the waist top layer web c1004c″.

FIG. 8d schematically illustrates an adhesive bonding step S1200. FIG. 8d shows how a second top layer web c1004b″ is fed in the machine direction MD together with a second set of continuous elastic strands c1400b. The continuous elastic strands c1400b of the second set are to be aligned in parallel with each other and substantially with the machine direction MD. Further, FIG. 8d shows how a set of one or more continuous additional elastic members c2500, c2600 are fed along the machine direction MD. The additional elastic members c2500, c2600 are to be positioned such that they extend at least partially in a direction not parallel with the machine direction. For example, and as illustrated in FIG. 8d, the additional elastic members c2500, c2600 may be arranged in an undulating pattern suitable for forming leg elastic members in body panels formed by the finalised combined laminate material web.

The second set of continuous elastic strands c1400b and the additional elastic members c2500, c2600 are sandwiched between the second portion of the combined bottom layer web c1040abc′ and the second top layer web c1004b″, and adhesive bonds are applied so as to form the second laminate web portion c1004b of the continuous laminate web material c1004 in an adhesive bonding step S1200.

The adhesive bonds may be applied to one or more out of the second top layer web c1004b″ and the combined bottom layer web c1040abc′, the second set of continuous elastic strands c1400b, and the set of additional elastic members c2500, c2600.

For example, and as described herein in relation to FIG. 9, a first adhesive 1080 may be applied substantially continuously to the second portion of the combined bottom layer web c1040abc′ that is to be covered by the second top layer web c1004b″. In addition, a second adhesive may be applied in defined regions 1082 of the second top layer web c1004b″ overlapping the additional elastic members c2500, c2600. Optionally, the second and first adhesives may be different, for example in that the second adhesive may be more fast curing than the first adhesive. Further, the adhesive bonding step may comprise applying adhesive to the second set of elastic strands c1400b.

The second top layer web c1004b″ has a third width W3 between a first longitudinal edge 1170 and a second longitudinal edge 1701. The second top layer web c1004b″ may be arranged such that the second longitudinal edge 1701 coincides with the second side edge 1501 of the combined bottom layer web c1040abc′. The third width W3 may be such that the second top layer web c1004b″ overlaps with the first top layer web c1004a″ in the connection region D.

Thus, a combined laminate web c1004 is formed having a first web portion c1004a having a width Wa, in this case corresponding to the first width W1 of the first top layer web c1004a″ minus the width WD of the connection region D and the width WE of the waist connection region E. It is to be noted that the first web portion c1004a encompasses the portions where the first top layer web c1004a″ forms a top side surface and the first bottom layer web c1004a′ forms a bottom side surface of the combined laminate web c1004. The combined laminate web c1004 further has a second web portion c1004b having a width Wb in this case corresponding to the third width W3 of the second top layer web c1004b″ minus the width WD of the connection region D. And finally, the combined laminate c1004 comprises a waist web portion c1004c having a width Wc corresponding the second width W2 of the folded waist top layer web c1004c″ minus the width WE of the waist connection region E.

FIGS. 8a, 8c, and 8d together illustrate another variant of a method of forming a combined laminate web 1004c, which in this case will have a cross-section such as illustrated in FIG. 7f.

In this method, the waist forming step S1150 is performed simultaneously with the welding step S1000 for forming the first web portion c1004a. In practice, the waist forming step S1150 and the welding step S1000 is thus performed in a united welding step S1000, S1150 in the manufacturing line, using the same welding equipment.

Thus, in the united welding step S1000, S1150 which is only schematically illustrated, the combined bottom layer web c1040abc′ is bonded to the first top layer web c1004a″ by a welded bond pattern, with the first set of elastomeric strands c1400a being sandwiched between the combined bottom layer web c1004bb′ and the first top layer web c1004a′, so as to be aligned in parallel with each other and substantially with the machine direction. Also, in the united welding step S1000, S1150 a first portion of the combined bottom layer web c1040abc′ is folded along the machine direction MD, such that the first portion forms, on each side of the fold, a waist bottom layer c1004c′ and a waist top layer c1004c″ in a waist web portion c1004c of the combined laminate web c1004. A waist portion set of continuous elastic strands c1400c is being advanced in a machine direction MD so as to be sandwiched between the waist bottom layer web c1004c′ and the waist top layer web c1004c″.

After the united welding step S100, S1150 is performed, the adhesive bonding step S1200 as described in relation to FIG. 8d in the above may be performed, so as to form the combined laminate material web c1004.

In the first variant of the method as illustrated by FIGS. 8a, 8b and 8d, the waist top layer 1004c″ is arranged to the top side of the first top layer 1004a″. Thus, the first top layer web c1004a″ is arranged adjacent the combined bottom layer web c1040abc′, whereafter the combined bottom layer web c1040abc′ is folded so as to form the waist portion c1004c with the waist top layer c1004c″ overlapping the first top layer web c1004a″ in the waist connection region E. Thereafter, the united welding step S100, S1150 is performed. The cross-section of the combined laminate web material c1004 is as illustrated in FIG. 7d.

In the second variant of the method as illustrated by FIGS. 8a, 8c and 8d, the waist top layer 1004c″ is arranged to the bottom side of the first top layer 1004a″. Thus, the combined bottom layer web c1040abc′ is first folded so as to form the waist portion c1004c with the waist top layer c1004c″, whereafter the first top layer web c1004a″ is arranged adjacent the combined bottom layer web c1040abc′, so as to overlap the waist top layer c1004c″ is the waist connection region E. Thereafter, the united welding step S1100, S1150 is performed. The cross-section of the combined laminate web material c1004 is as illustrated in FIG. 7f.

However, the arrangement of the webs resulting in the different cross-sections as illustrated in FIGS. 8d and 4f may be selected as desired regardless of whether the welding is performed in a welding step and a waist forming step being performed as separate steps, or as a united step.

In the welding bonding steps and the adhesive bonding step as described in the above, the steps are performed such that the respective set of elastomeric strands is attached between the respective top and bottom layers when in a stretched state in relation to the respective top and bottom layers. For example, the elastomeric strands may be stretched so as to be elongated to a length being at least 2× a relaxed length of the elastomeric strands, such as to a length being at least 3× a relaxed length of the elastomeric strands. The respective top and bottom layers may be stretched only slightly, i.e., to an amount required to perform the feeding operation. As such, the elastomeric strands are attached when in a stretched state in relation to the respective top and bottom layer. Accordingly, in a relaxed state of the combined laminate web, wherein the elastic strands are contracted, the top and bottom layers may form wrinkles along the elastic strands. The combined laminate web is stretchable along the machine direction from the relaxed state to a stretched state, in which the wrinkles of the top and bottom layers may be reduced or significantly completely flattened out. Thus, the required elasticity of the combined laminate web along the machine direction is achieved.

The combined laminate material web c1004 may be subject to additional manufacturing steps before being fed to a method for forming an absorbent article.

For example, zones of reduced elasticity may be formed with regular intervals along the combined laminate material web c1004. In particular, such zones of reduced elasticity may be formed where the absorbent assemblies 7 of the articles 1100 to be formed are to overlap with the combined laminate material web c1004. In zones of reduced elasticity, the elasticity along the machine direction provided by the sets of elastic strands is removed or significantly reduced.

To provide zones of reduced elasticity in the combined laminate material web c4, known methods involving for example the inactivation or removal of elastomeric strands in the zones. For example, the methods may involve the cutting or destruction of any elastomeric strands in the zones.

### Method for Forming Absorbent Articles

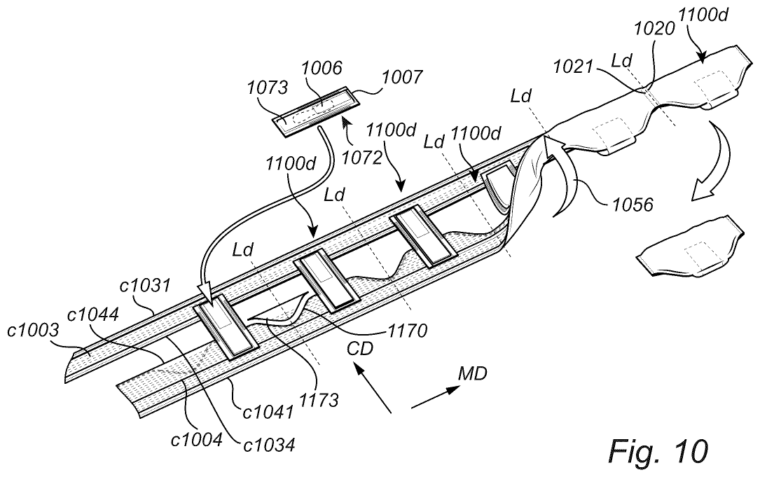

FIG. 10 schematically illustrates a method for producing a disposable pant-type article 1001 of the variant with the combined panel, such as an article as exemplified in the above in conjunction with FIG. 7a, The method of FIG. 10 comprises the steps, and optionally the optional steps, described above for the method for producing a disposable absorbent pant-type article, as described in conjunction with FIG. 6.

The method comprises providing a rear body panel web c1004 and a front body panel web c1003, wherein at least one out of the rear body panel web c1004 and the front body panel web c1003 is a combined laminate material web, for example as exemplified in the above.

As illustrated in the variant of FIG. 10, the method may comprise feeding a continuous front panel web c1003 and a continuous rear panel web c1004 in the machine direction MD, the front panel web c1003 and the rear panel web c1004 being spaced apart in the cross-direction Cd.

The front panel web c1003 has a front waist edge c1031 and an opposing front crotch edge c1034. The rear panel web c1004 has a rear waist edge c1041 and an opposing rear crotch edge c1044.

The rear panel web c1004 is spaced apart from the front panel web c1003 in a cross-direction CD, perpendicular to the machine direction MD, with a distance between the front panel web c1003 and the rear panel web c1004 in the cross-direction CD between the crotch edge c1034 of the front panel web c1003 and the crotch edge c1044 of the rear panel web c1004.

The front panel web c1003 and the rear panel web c1004 in the spaced apart condition together form a composite web 1000 for forming interconnected precursor articles 1100a-1100e.

As illustrated in FIG. 10, the method further comprises applying and attaching absorbent assemblies 7 to the moving composite web 1000 of interconnected precursor articles 1100a-1100e. Each absorbent assembly 1007 comprises an absorbent core 1006, a liquid impermeable backsheet 1072 and a liquid permeable topsheet 1073, the absorbent core 1006 being sandwiched between the backsheet 1072 and the topsheet 1073.

Each absorbent assembly 1007 is applied to the composite web 1000 of precursor articles on a precursor article centerline extending in the cross-direction, CD.

The absorbent assemblies 7 are applied spaced-apart in the machine direction, MD, and are attached with a front end part 1007f of each absorbent assembly 1007 overlapping a front panel zone of the front body panel web c1003 and with a rear end part 1007r of each absorbent assembly 1007 overlapping a rear panel zone of the rear body panel web c1004. The front panel zone and/or the rear panel zone may be provided with reduced elasticity, as mentioned in the above. For example, in the front panel zone and/or the rear panel zone, any elastic strands may be cut so as to disable the elasticity in the zones.

The absorbent core 1006, the backsheet 1072 and the topsheet 1073 are of the kinds described above.

### Leg Cuts

As illustrated in FIG. 10, after the application and attachment of an absorbent assembly 6 to the composite web 1000 of interconnected precursor articles 1100a-1100e, leg cuts 1170 may be formed in the composite web 1000 of interconnected precursor articles 1100a-1100e between the precursor articles 1100a-1100e, each leg cut 170 having a curved configuration as indicated between the precursor articles 1100d and 1100e in FIG. 10. The leg cuts form a first leg edge 1018 of the leading precursor article 1100d and a second leg edge 1019 of the trailing precursor article 1100e.

After forming the leg cuts 1170, the cut-out portions 1173 are removed from the composite web 1100 of interconnected precursor articles 1100a-1100e and are not further part of the process as disclosed herein.

After forming the leg cuts, the absorbent assemblies 1006 will be located centrally between the first leg edge 1018 and the second leg edge 1019 of each precursor article 1100a-100e in the composite web 1000 of interconnected precursor articles.

As is shown in FIG. 10, when the leg edges 1018, 1019 have been formed, the composite web 1000 of interconnected precursor articles 1100a-1100e is folded along a fold-line extending in the machine direction MD to bring the waist edge c1031 of the front panel web c1003 into alignment with the waist edge c1041 of the rear panel web c1004.

This corresponds to how the leg cuts are formed in the method depicted in FIG. 6.

### Side Seams

A first side seam 1020 and a second side seam 1021 are formed in each precursor article 1100a 100e along the dividing lines Ld between the precursor articles 1100a-1100e, from the aligned waist edges c1031, c1041 of the front and rear panel webs c1003, c1004, to the leg cuts 1170.

The first side seam 1020 of a leading precursor article 1100a and the second side seam 1021 of a trailing precursor article 1100b may be formed as a single broad seal extending along the dividing line Ld, as shown in FIG. 10. When cutting away individual pant-type articles from the chassis web 1100 of precursor articles 1100a-1100e by severing the composite web 1000 of interconnected precursor articles 1100a-1100e along the dividing lines Ld, the single broad seal shown in FIG. 10 will at the same time be divided into the two side seams 1120, 1121 of the interconnected precursor articles 1100a, 1100b.

Alternatively, the side seams 1120, 1121 may be formed as individual seals placed on each side of the dividing line Ld.

The side seams may be formed by any method suitable for the purpose, such as by thermo-welding, ultrasonic welding or by means of adhesive. In case of adhesive side seals, the adhesive is generally applied before the chassis web is folded along the folding line Lv.

### Waist

With reference to FIG. 9, the front body panel web c1003 and the rear body panel web c1004 are each formed so as to comprise an integral waist panel portion 1003c, 1004c. As mentioned in the above, the front body panel web c1003 and/or the rear body panel web c1004 may comprise other types of waist features. For example, the front body panel web c1003 and/or the rear bod panel web c1004 may be provide with a waist feature in the form of a pre-fabricated band-shaped waist elastic member which may comprise one or more elastic elements such as bands, strings, elastic film, elastic nonwoven, etc. Such a prefabricated band-shaped waist elastic member may be applied to each of the front body panel web c1003 and/or the rear body panel web c1004 prior to application of the absorbent assemblies 1006 to the precursor web 1100, as illustrated in FIG. 10.

For example, a waist feature may be applied along an edge of the front panel web and/or the rear panel web before or after cutting the precursor web into individual articles.

It is also to be understood that waist elastic may be added at the waist edges of a composite web of precursor articles at any stage of the process up until severing of individual absorbent pant-type articles from the composite web of precursor articles.

The pant-type articles as disclosed herein may be of the kind shown in the Figures and having a three-part chassis with an absorbent assembly or crotch panel being connected to a front panel and which is connected to a rear panel, such that the absorbent assembly extends longitudinally to bridge between the front panel and the rear panel. Alternatively, the pant-type article may have a unitary chassis having a combined laminate web extending the full distance between the front panel waist edge and the rear panel waist edge, the combined laminate web constituting at least a part of each of the front panel and the rear panel and constituting a crotch web material in the crotch region of the pant-type article.

In such an alternative embodiment, a combined laminate web may be formed, which combined laminate web may form both the front body panel 1003 and the rear body panel 1004 of the absorbent article. In this case, the combined laminate web may comprise at least a front body panel section and a rear body panel section. Still such a combined laminate web may comprise at least a first web portion and a second web portion as defined in the above examples. After the application and attachment of an absorbent assembly 6 to such a composite web, leg cuts 1170 may be formed in the composite web 1000 of interconnected precursor articles 1100a-1100e between the precursor articles 1100a-1100e, each leg cut 170 having a closed loop configuration as indicated between the precursor articles 1100b and 1100c in FIG. 10. As such, the closed-loop leg cuts 1170 may be formed so as to provide a first front leg edge and a first rear leg edge of the leading precursor article, and a second front leg edge and a second rear leg edge of the trailing precursor article.

However, methods involving a unitary chassis being formed by a combined laminate web may require more use of material than the three-part chassis solutions.

### Weld Bonding

Numerous techniques involving welded bond patterns are known which may be used for attaching the first set of elastomeric strands 1400a between the first bottom layer 1004a′ and the first top layer 1004a″ in the first panel portion 1004a/first web portion c1004a.

For example, the welded bond pattern may comprise pairs of adjacent weld bonds, the adjacent weld bonds being provided on each side of an elastomeric strand, for attaching each of the first set of elastomeric strands 1400a.

In some variants, such pairs of adjacent weld bonds are applied so as to retain the elastomeric strand primarily by friction and/or clamping the elastomeric strand.

For example, the pairs of adjacent weld bonds are arranged such that, each of the elastomeric strands is anchored in a passage defined between such a pair of adjacent weld bonds, wherein a distance between the adjacent weld bonds across the passage is smaller than the diameter of the elastomeric strand when in a non-tensioned state. The elastic strands are applied between the top and bottom layers in a stretched state and the top and bottom layers are bonded to each other while the elastic strands are held in the stretched state. In the stretched state, the elastic strands have a smaller diameter than in a relaxed or non-tensioned state. Thereby, the elastic strands will be mechanically trapped between the bonds in the pairs of adjacent bonds as the stretched elastic strands are allowed to relax and resume their non-tensioned diameter.

In some variants, the application of such pairs of adjacent weld bonds involves melting of at least one out of the top layer, the bottom layer and the elastic strands, such that the pairs of adjacent weld bonds also involve a fusion connection between the top and/or bottom layer and the elastic strand.

The bonding pattern may be formed by ultrasonic bonding or heat bonding, the bond elements being formed by locally heating the layers which are bonded together to cause thermoplastic material in the laminated layers to soften or melt and subsequently consolidate in the bonds.

Advantageously, the welded bond pattern comprises ultrasonic bonds. The welded bond pattern may for example be created using a bonding system such as described in e.g. EP 4 025 412 A1 or EP 3 519 162.

The bond pattern may comprise a plurality of discrete bonds, the bonds being arranged in a regular bonding pattern. For example, the discrete bonds may form a pattern of uniformly distributed discrete bonds.

The bonding elements may be distributed over the body panel in the regular bonding pattern over the full area of the body panel, i.e., also in any zones with reduced elasticity. In other words, the bonding pattern is the same in the body panel zone with reduced elasticity as in the other parts of the body panel. Instead, the reduced elasticity may be accomplished as mentioned herein by e.g. cutting or destroying the elastic strands.

The bond pattern may comprise a plurality of discrete bonds. The bonding pattern may be uniform all over the first portion 1004a of the rear body panel 1004.

The bond pattern may comprise a plurality of discrete bonds being arranged for example along lines or curves. For example, the bond pattern may comprise a plurality of discrete bonds being arranged so as to form lines forming an angle with the longitudinal direction L/cross-direction CD. Alternatively, the bond pattern may comprise a plurality of discrete bonds being arranged so as to follow undulating lines.

### Adhesive Bonding

The second panel portion 1004b is bonded by adhesive bonds, wherein the adhesive bonds are applied to one or more out of the second bottom layer 1004b′, the second top layer 1004b″, the second set of elastomeric strands 1400b, and the leg elastic members 1025, 1026. To this end, prior known adhesive techniques may be used.

In FIG. 9, illustrating a combined laminate web, a variant of an adhesive pattern is shown. The same pattern may be applied to the article of FIG. 7a, although it is not shown here for reasons of clarity of the drawings.

As seen in FIG. 9, adhesive is substantially continuously applied over the surface of the second top layer 1004b″ which is facing the second bottom layer 1004b′, as illustrated by the dashed region 80.

In addition, adhesive is applied only in defined regions 1082 of the surface of the second bottom layer 1004b′ which is facing the second top layer 1004b″. The defined regions 1082 have a size and distribution so as to overlap the additional elastic members c2500, c2600 i.e. the leg elastic members 1025, 1026 in the article 1001 in the second panel portion 1004b. Finally, in the illustrated variant, also the second set of elastomeric strands 1400b are provided with adhesive.

For example, a second adhesive may substantially continuously over the second top layer 1004b″ and a first adhesive may be applied in the defined regions of the second bottom layer 1004b′. For example, the second adhesive may be different from the first adhesive in that the second adhesive is more fast consolidating than the first adhesive. The first adhesive may be intended for attachment of elastic members and the second adhesive may be intended for lamination of webs.

### Materials

At least one, preferably each, bottom layer 4, the top layer 1004b, and optionally the waist bottom layer 1004c′, and the waist top layer 1004c″, comprises or consists of a sheet of a nonwoven material. In the variant comprising a combined panel, at least one, preferably each, out of the second bottom layer 1004b′, the second top layer 1004b″, the first bottom layer 1004a′, the first top layer 1004a″, and optionally the waist bottom layer 1004c′, and the waist top layer 1004c″ comprises or consists of a sheet of a nonwoven material.

The nonwoven material may contain thermoplastic fibers, preferably the nonwoven material contain at least 50% thermoplastic fibres and more preferably at least 80% thermoplastic fibres.

The nonwoven material layers or webs may be of any suitable nonwoven material such as spunbond, air laid, wet laid, carded, electro spun or meltblown nonwovens. The nonwoven material may be bonded by any suitable technique, such as by needling, hydroentangling, ultrasonic welding, or thermobonding.

The fibers of the nonwoven materials used herein may be man-made fibers, natural fibers or mixtures of man-made and natural fibers. Man-made fibers include mono-component, bi-component and multicomponent fibers of polymers such as polyolefins, polyesters, polyacrylates, etc., as well as regenerated fibers such as viscose fibers and modal fibers. Natural fibres are for instance cellulosic fibres such as pulp fibers, cotton fibers, flax, hemp, etc.

For layers that are bonded by ultrasonic bonding or heat bonding, the nonwoven materials which are used to form the layers should contain a sufficient amount of thermoplastic fibres to allow the bond elements to be formed. Examples of suitable thermoplastic polymers for use in the nonwoven webs as disclosed herein are polyethylene polypropylene and other polyolefin homopolymers and copolymers and polyesters. The weldable nonwoven webs may have a high content of thermoplastic component and preferably contain at least 50% thermoplastic fibres and more preferably at least 80% thermoplastic fibres.

The nonwoven materials may have a basis weight of from 8 to 26 gsm, such as from 10 to 22 gsm.

The elastic material in the elastomeric strands and/or the leg elastic members arranged may be any suitable elastic material such as natural or synthetic rubber, thermoplastic elastomers, such as thermoplastic polyurethane or styrene block co-polymers or elastane, also referred as to spandex polyurethane-polyurea copolymer. The elastic strands and/or the elastic elements of the leg elastic members may be of the elastane type that is available under the trade name “LYCRA”, but any suitable elastic thread may be used.

The threads may have a linear mass density, dtex, of about 80-1200 dtex.

The number of elastomeric strands in each panel portion, and the longitudinal spacing between the strands in each panel portion, may be selected so as to provide the panel with suitable elasticity and a good fit.

For example, and as in the illustrated variants, the longitudinal spacing between the strands in the second set of elastomeric strands 1400b in the second panel portion 1004b may be no less than the longitudinal spacing between the strands in the first set of elastomeric strands 1400a in the first panel portion 1004a.

For example, the longitudinal spacing between the strands in the first set of elastomeric strands 1400a of the first panel portion 1004a may be from 2 to 20 mm, and/or the longitudinal spacing between the strands in the second set of elastomeric strands 1400b may be 2 to 35 mm, and/or the longitudinal spacing between the strands in the third set of elastomeric strands may be 2 to 12 mm.

While examples of different variants have been described in the above with reference to the drawing, other variants and modifications will readily occur to those skilled in the art. For example, shapes and sizes of the absorbent articles are not limited to the shapes and sizes shown in the drawings. Moreover, features and explanations as described in relation to one of the variants disclosed herein may be applied to or combined with other variants disclosed herein.

1-37. (canceled)

38. A disposable absorbent pant-type article having a longitudinal extension along a longitudinal direction and a transversal extension along a transversal direction, the article comprising
a front section,
a back section, and
a crotch section, being located between and joining the front section and the back section,
wherein an absorbent assembly is arranged with a central part of the absorbent assembly being located in the crotch section;
the front section and the back section being formed by separate panels;
the front section being joined to the back section by a first front section side edge being joined to a first back section side edge in a first side seam, and a second front section side edge being joined to a second back section side edge in a second side seam;
such that the article forms
a waist opening, defined by a waist edge of the front section and a waist edge of the back section,
a first leg opening, defined by a first leg edge of the front section and a first leg edge of the back section, and
a second leg opening, defined by a second leg edge of the front section and a second leg edge of the back section;
at least one of the front section and the back section comprises a first leg elastic member, arranged along the first leg edge of the at least one of the front section and the back section, and a second leg elastic member, arranged along the second leg edge of the at least one of the front section and the back section,
wherein each leg elastic member comprises one or more elastic elements extending at least partially in the longitudinal direction,
the first and second leg elastic members are sandwiched between a first layer and a second layer comprised in the at least one of the front section and the back section, which first and second layers are bonded to each other,
the at least one of the front section and the back section further comprises a set of elastic strands extending substantially or completely in the transversal direction of the article, the first leg elastic member ends at or in the vicinity of the first leg edge, and the second leg elastic member ends at or in the vicinity of the second leg edge, the first and the second leg elastic members not being included in the side seams.

39. The article according to claim 38, wherein the first leg elastic member and the second leg elastic member end with a respective cut end.

40. The article according to claim 38, wherein the first and second layers are bonded to each other by an adhesive located on one or more of the first layer, the second layer and the first and second leg elastic members.

41. The article according to claim 38, wherein the first and second layers are bonded to each other by means of welding.

42. The article according to claim 38, wherein the first and second layers comprised in the at least one of the front section and the back section are bonded to each other by adhesive located on one or more of the elastic strands of the set elastic strands and/or by a welded bond pattern associated with one or more of the elastic strands of the set elastic strands, the set of elastic strands being sandwiched between the first and second layers.

43. The article according to claim 38, wherein the at least one of the front section and the back section further comprises
a first leg top elastic strand, extending substantially or completely in the transversal direction, and ending at the first leg edge in a longitudinal position being between the first leg elastic member and the first side seam, and
a second leg top elastic strand, extending substantially or completely in the transversal direction and ending at the second leg edge in a longitudinal position being between the second leg elastic member and the second side seam.

44. The article according to claim 38, wherein the at least one of the front section and the back section further comprises
a first leg side elastic strand, extending substantially or completely in the transversal direction and crossing the first leg elastic member, and
a second leg side elastic strand, extending substantially or completely in the transversal direction and crossing the second leg elastic member.

45. The article according to claim 43, wherein the first and second leg top elastic strands and/or the first and second leg side elastic strands form part of, or constitute, the set of elastic strands extending substantially or completely in the transversal direction of the article.

46. The article according to claim 38, wherein the first leg edge has a longitudinal extension, the first leg elastic member ending at a longitudinal position of the first leg edge being located within a range of from 1% to 60%of the longitudinal extension, as determined from a lower end of the first side seam, and
wherein the second leg edge has a longitudinal extension, the second leg elastic member ending at a longitudinal position of the second leg edge being located within a range of from 1% to 60%of the longitudinal extension, as determined from a lower end of the second side seam.

47. The article according to claim 38, wherein the first leg elastic member ends at a longitudinal position of the first leg edge being located within a range of from 1 mm to 80 mm, as determined from the lower end of the first side seam, and
wherein the second leg elastic member ends at a longitudinal position of the second leg edge being located within a range of 1 mm to 80 mm, as determined from the lower end of the second side seam.

48. The article according to claim 38, wherein the first leg elastic member extends along a distance being within a range of 50%-99% of a length of the first leg edge, and wherein the second leg elastic member extends along a distance being within a range of 50%-99% of a length of the second leg edge.

49. The article according to claim 38, wherein the first leg elastic member extends a distance being within a range of 150-300 mm along the first leg edge and wherein the second leg elastic member extends a distance being within a range of 150-300 mm along the second leg edge.

50. The article according to claim 38, wherein the first leg elastic member extends at least from the absorbent assembly to the first leg edge and
wherein the second leg elastic member extends at least from the absorbent assembly to the second leg edge.

51. The article according to claim 38, wherein the first leg elastic member extends along a longitudinal length being less than 40% of a total longitudinal length of the at least one of the front section and the back section, and
the second leg elastic member extends along a longitudinal length being less than 40% of the total longitudinal length of the at least one of the front section and the back section.

52. The article according to claim 38, further comprising a first and a second crotch elastic member, extending along a respective longitudinal side of the absorbent assembly.

53. The article according to claim 38, wherein the at least one of the front section and the back section comprises a combined panel, wherein the combined panel comprises, as seen along the longitudinal direction, a first panel portion and a second panel portion, the first panel portion and the second panel portion being connected by a connection region, and
the first panel portion comprises a first bottom layer and a first top layer being superimposed such that, in the first panel portion, the first bottom layer forms a bottom side surface of the combined panel and the first top layer forms a top side surface of the combined panel,
the first panel portion further comprises at least a subset of the set of elastic strands being sandwiched between the first bottom layer and the first top layer, the elastic strands being aligned substantially in parallel with each other and with the transversal direction, and the elastic strands being spaced apart from each other in the longitudinal direction, and
the first panel portion is bonded by the first bottom layer being bonded to the first top layer by a welded bond pattern attaching the at least a subset of the set of elastic strands between the first bottom layer and the first top layer,
the second panel portion comprises a second bottom layer and a second top layer being super-imposed such that, in the second panel portion, the second bottom layer forms a bottom side surface of the combined panel and the second top layer forms a top side surface of the combined panel, wherein the first leg elastic member and the second leg elastic member are sandwiched between the second bottom layer and the second top layer, and the second panel portion is bonded by the second bottom layer being bonded to the second top layer by adhesive bonds, attaching the first leg elastic member and the second leg elastic member between the second bottom layer and the second top layer.

54. A method for producing a disposable absorbent pant-type article, the article comprising a front section, a back section, and a crotch section being located between the front section and the back section, wherein an absorbent assembly is arranged with a central part of the absorbent assembly being located in the crotch section, the front section and the back section being formed by separate panels, the method comprising:
feeding a bottom layer web in the machine direction,
feeding a top layer web in the machine direction,
feeding a set of continuous elastic strands in the machine direction,
feeding a continuous leg elastic member comprising one or more elastic elements,
positioning the continuous leg elastic member, such that it extends at least partly in an extension direction being both in the cross direction and the machine direction,
bonding the bottom layer web to the top layer web to form a first laminate web material, thereby sandwiching the set of continuous elastic strands between the bottom layer web and the top layer web, such that the elastic strands of the set of continuous elastic strands are aligned substantially in parallel with each other and with the machine direction, and being spaced apart from each other in the cross direction and sandwiching the continuous leg elastic member between the bottom layer web and the top layer web,
positioning the first laminate web material in a position corresponding to one of a front section or a back section of the article,
providing a second laminate web material and positioning the second laminate web material in a position corresponding to the other of the front section or the back section of the article,
intermittently providing absorbent assemblies spaced apart in the machine direction and positioning them with a first end portion in overlap with the first laminate web material and a second end portion in overlap with the second laminate web material, thereby forming a composite web of interconnected precursor articles,
making leg cuts going from a first side edge of the first laminate web material, thereby cutting through the continuous leg elastic member, the leg cuts being positioned in between the absorbent assemblies as seen in a machine direction,
folding the composite web along a fold line extending in the machine direction, thereby bringing a first edge of the composite web into alignment with a second edge of the composite web,
forming first and second side seams in each precursor article, thereby joining the first laminate web material with the second laminate web material, the side seams being formed in the cross direction along dividing lines between the precursor articles, and
severing individual disposable absorbent pant-type articles from the composite web of precursor articles.

55. The method according to claim 54, wherein the continuous leg elastic member is positioned such that it extends for at least 10 mm in a direction parallel with or substantially parallel with the machine direction in a first zone along and adjacent to the first edge of the laminate web material, at least 10 mm in a direction parallel with or substantially parallel with the machine direction in a second zone spaced apart from the first zone, and both in the cross direction and the machine direction in a transfer zone located between the first and second zones.

56. The method according to claim 54, wherein the continuous leg elastic member is positioned such that it extends over less than 40% of the width of the first laminate web material.

57. The method according to claim 54, wherein the continuous leg elastic member is positioned in relation to the set of continuous strands, such that it crosses at least one elastic strand of the set of continuous elastic strands.

58. The method according to claim 54, wherein the continuous leg elastic member is positioned in relation to the set of continuous strands, such that at least one elastic strand of the set of continuous elastic strands ends at the leg cut without crossing the continuous leg elastic member.

59. The method according to claim 54, wherein bonding the bottom layer web and the top layer web comprises bonding by applying adhesive to form adhesive bonds and/or by performing welding.

60. The method according to claim 59, wherein bonding the bottom layer web and the top layer web comprises applying adhesive to one or more of the bottom layer web, the top layer web, the continuous leg elastic member and the set of continuous elastic strands.

61. The method according to claim 54, wherein bonding the bottom layer web and the top layer web comprises applying adhesive to one or more of the bottom layer web and the top layer web in defined regions overlapping the continuous leg elastic member.
